(19) United States
(12) Patent Application Publication
(10) Pub. No.: US 2025/0066826 A1
(43) Pub. Date: Feb. 27, 2025

(54) ARTIFICIAL SYNTHESIS METHOD FOR MALONYL-COENZYME A (COA) AND USE THEREOF

(71) Applicant: SHANGHAI JIAO TONG UNIVERSITY, Shanghai (CN)

(72) Inventors: Zaigao TAN, Shanghai (CN); Tiangang LIU, Shanghai (CN); Jian LI, Shanghai (CN)

(73) Assignee: SHANGHAI JIAO TONG UNIVERSITY, Shanghai (CN)

(21) Appl. No.: 18/801,905

(22) Filed: Aug. 13, 2024

Related U.S. Application Data
(63) Continuation of application No. PCT/CN2023/113928, filed on Aug. 21, 2023.

(51) Int. Cl. C12P 11/00; C12N 9/02; C12N 9/10; C12N 15/70; C12P 7/22; C12P 7/40; C12P 17/06

(52) U.S. Cl. CPC C12P 11/00; C12N 9/0004; C12N 9/1096; C12N 15/70; C12P 7/22; C12P 7/40; C12P 17/06; C12Y 206/01018

(57) ABSTRACT

An artificial synthesis method for malonyl-CoA and use thereof are provided. By means of heterologous expression of an aminotransferase and a malonyl-CoA reductase, an artificial synthesis pathway for synthesizing malonyl-CoA by using β-alanine (β-ala) as a precursor is constructed as follows: firstly, under catalysis of a transaminase, β-ala transfers amino groups to α-ketonic acid (such as pyruvic acid, oxaloacetic acid, or α-ketoglutaric acid, etc.), to form an intermediate product 3-oxopropanoate and a corresponding amino acid; the 3-oxopropanoate generates malonyl-CoA under the action of the malonyl-CoA reductase. This pathway addresses the defects of the natural malonyl-CoA synthesis pathway, such as low carbon utilization, consumption of energy substance ATP, release of greenhouse gas CO₂, and strict regulation of pathway enzymes, a pyruvate dehydrogenase (PDH) and an acetyl-CoA carboxylase (ACC), thereby achieving high yielding of products using malonyl-CoA as a precursor, including flaviolin, octanoic acid, phloroglucinol, pentadecaheptaene, natamycin, and spinosad.

### CROSS REFERENCE TO THE RELATED APPLICATIONS

This application is a continuation application of International Application No. PCT/CN2023/113928, filed on Aug. 21, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBDD112-PKG_Sequence_Listing.xml, created on Jul. 3, 2024, and is 152,864 bytes in size.

### TECHNICAL FIELD

The present invention relates to the field of biotechnology, in particular, to an artificial synthesis method for malonyl-coenzyme A (CoA) and use thereof.

### BACKGROUND

Malonyl-CoA is considered as a central compound in the metabolic activity of life. In primary metabolism, malonyl-CoA is an essential extension unit for the synthesis of long-chain fatty acids, which are precursors of cell membrane phospholipids. In addition, after the esterification reaction between the long-chain fatty acids and methanol/ethanol, the fatty acid methyl ester/ethyl ester formed is the main component of biodiesel. Compared with traditional petroleum diesel, the biodiesel has many significant advantages such as renewable raw materials and environmental friendliness, and is considered as a new generation of bioenergy.

In secondary metabolism, malonyl-CoA is a precursor for the synthesis of almost all polyketides and flavonoids (FIG. 1A). Currently, over 10000 different polyketides have been discovered, and countless new products have been derived from them. According to statistics, over 20 types of polyketides (such as erythromycin, tetracycline, lovastatin, and abamectin) have become commercial drugs. The erythromycin is the most commonly used macrolide antibiotic in medicine and animal husbandry, and statins, including lovastatin, account for over 80% of the total market for lipid-lowering drugs. At present, the global annual sales of these over 20 types of drugs have exceeded 20 billion dollars. The flavonoids are also widely existed secondary metabolites. Currently, over 8000 types of flavonoids have been discovered, most of which have good antiviral, antibacterial, anti-inflammatory, anticancer, anti-obesity and other physiological activities. They have broad application prospects in health products, cosmetics, medicines, and the like. It is expected that the market of the flavonoids will reach 1.2 billion dollars by 2024.

With the development of synthetic biology and metabolic engineering, constructing efficient microbial cell factories to synthesize high-value-added malonyl-CoA derivatives (long-chain fatty acids, polyketides, flavonoids, etc.) has become a research hotspot in the field of biosynthesis (FIG. 1A). However, under normal conditions, the synthesis of malonyl-CoA in microbial cells is relatively low, always at a low level, and malonyl-CoA cannot be added exogenously, thus becoming a key bottleneck limiting the efficient synthesis of derivatives. On this basis, enhancing the supply capacity of intracellular malonyl-CoA is crucial for achieving efficient biosynthesis of derivatives. At present, the known natural synthesis pathways of malonyl-CoA mainly include PDH-ACC pathway and MCS pathway. The following will explain these two pathways separately:

1. PDH-ACC pathway: this is the most widely-present and widely-used malonyl-CoA synthesis pathway that is almost present in all cells. In this pathway, pyruvic acid produced by the decomposition and metabolism of a carbon source first generates acetyl-CoA, NADH, and CO₂ under the catalysis of a pyruvate dehydrogenase complex (PDH), and then, acetyl-CoA, ATP, and HCO₃⁻ finally form malonyl-CoA under the catalysis of acetyl-CoA carboxylase (ACC) (FIG. 1B).

Although it is the most important malonyl-CoA synthesis pathway in cells, this pathway has obvious catalytic defects: firstly, the decarboxylation reaction catalyzed by PDH generates greenhouse gases (CO₂), while also losing one molecule of carbon, which reduces atomic economy (FIG. 1B). Secondly, the PDH complex is one of the most complex enzymes in nature, consisting of three different subunits: pyruvate dehydrogenase (E1), dihydrolipoyl transacetylase (E2), and dihydrolipoyl dehydrogenase (E3). In Gram-negative bacteria, the typical composition of this complex is ([E1]₂₄[E2]₂₄[E3]₁₂), with a molecular weight of approximately 4600 kDa; however, in Gram-positive bacteria and eukaryotes, the typical composition of this complex is ([E1]₃₀[E2]₆₀[E3]₁₂), with a molecular weight of approximately 8,000 to 10,000 kDa. Modifying and overexpressing such a complicated complex would impose a huge burden on cells. Thirdly, PDH catalysis requires various cofactors such as thiamine pyrophosphate (TPP), lipoic acid, FAD, NAD⁺, CoA, etc. These cofactors are also required for the catalysis of other intracellular enzymes. Therefore, the synthesis of malonyl-CoA is very prone to being highly competitive with other intracellular metabolisms. In addition, PDH catalysis is strictly inhibited by acetyl-CoA, NADH, etc., where acetyl-CoA is considered as a typical PDH inhibitor: 2 mM acetyl-CoA can lead to a 70% decrease in PDH activity. Finally, PDH activity is also affected by the intracellular energy state, and a high ATP level severely inhibits PDH activity: when ADP/ATP is equal to 1, PDH activity decreases by 90%.

ACC consists of three subunits: biotin carboxylase (BC), carboxyl transferase (CT), and biotin carboxyl carrier protein (BCCP). In addition to the catalytic defects of PDH mentioned above, ACC catalysis also has obvious defects: firstly, ACC catalysis requires the consumption of additional ATP, which wastes intracellular energy (FIG. 1B). Secondly, the transcription of ACC coding genes is strictly regulated by cells, and bacterial cell growth enters a stable phase, while mRNA transcription tends to stop. Thirdly, ACC catalysis has low efficiency, and is susceptible to strict inhibition by various metabolites/enzymes such as long-chain acyl-CoA, cyclic adenosine monophosphate (cAMP), AMP-dependent protein kinase (AMPK), and various ions such as Mg²⁺ and HCO₃⁻ (FIG. 1B). The conformational inhibition of long-chain acyl-CoA on ACC is considered as a classic case of conformational inhibition: an extremely low concentration of long-chain acyl-CoA, such as 0.24 M palmitoyl-CoA (C16-CoA), can lead to a loss of over 80% of ACC activity.

2. MCS pathway: this pathway has a narrow present scope, and is mainly present in cells such as rhizobia, Arabidopsis, Pseudomonas fluorescens, etc., and mitochondria in mammals. In this pathway, malonate forms malonyl-CoA under the catalysis of malonyl-CoA synthetase (MCS) (FIG. 1C). The main defects of this pathway include: firstly, the MCS catalytic process requires the consumption of ATP energy. Secondly, this pathway requires the addition of an additional substrate malonate, which increases the cost of biosynthesis. Thirdly, extracellular malonate requires specialized transport of exogenous membrane transport proteins (such as MatC) to be transferred into cells, and exogenous membrane proteins generally face problems such as difficulty in soluble expression and misfolding in microbial cells. Finally, malonate is a structural analogue of succinic acid, and is a competitive inhibitor of succinate dehydrogenase in key central carbon metabolism activity—TCA cycle; adding malonate will disrupt the intracellular normal TCA cycle, causing serious impacts on cell survival and growth. The existence of this series of defects has also resulted in minimal effectiveness in the modification of improving microbial intracellular malonyl-CoA levels by introducing exogenous MCS pathways.

### SUMMARY

In view of this, the present invention aims to provide an artificial synthesis method for malonyl-CoA and use thereof. According to the artificial synthesis method for malonyl-CoA provided by the present invention, by means of heterologous expression of an aminotransferase and a malonyl-CoA reductase, an artificial synthesis pathway for synthesizing malonyl-CoA by using β-alanine (β-ala) as a precursor is constructed as follows: firstly, under catalysis of the aminotransferase, β-ala transfers amino groups to α-ketonic acid (such as pyruvic acid, oxaloacetic acid, or α-ketoglutaric acid), to form an intermediate product 3-oxopropanoate and a corresponding α-amino acid; the 3-oxopropanoate generates malonyl-CoA under the action of the malonyl-CoA reductase. This pathway addresses the defects of the natural malonyl-CoA synthesis pathway, such as low carbon utilization, consumption of energy substance ATP, release of greenhouse gas CO₂, and strict regulation of pathway key enzymes, a pyruvate dehydrogenase (PDH) and an acetyl-CoA carboxylase (ACC), thereby achieving improvement on efficient synthesis of compounds using malonyl-CoA as a precursor, including flaviolin, octanoic acid, phloroglucinol, pentadecaheptaene, natamycin, and spinosad. After introducing the artificial malonyl-CoA synthesis pathway, the tolerance of cells to a series of stress conditions such as organic acids, osmotic pressure, and cytotoxic substances is improved.

The technical solution provided by present invention is as follows:

### First Aspect

The present invention provides an artificial synthesis method for malonyl-CoA, including the following steps:
S1, forming 3-oxopropanoate and a compound represented by formula (2) by β-alanine and a compound represented by formula (1) under catalysis of an aminotransferase,

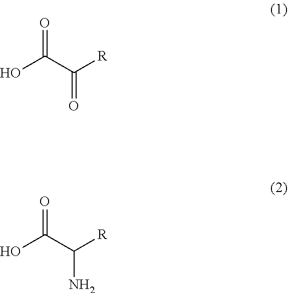

wherein R is selected from H, alkanes, alcohols, and alkanoic acids; and
S2, forming malonyl-CoA and 2[H] by the 3-oxopropanoate, CoA, and an electron acceptor by transferring electrons of the 3-oxopropanoate to the electron acceptor under catalysis of an oxidoreductase.

The electron acceptor includes NAD⁺, NCD⁺, NTD⁺, NGD⁺, FAD, CoQ (ubiquinol), PQ (plastoquinol), FMN, NADP⁺, NCDP⁺, NGDP⁺, NTDP⁺, oxidized ferredoxin, oxidized cytochrome c, oxidized thioredoxins, oxidized glutaredoxins, O₂, etc.

The 2[H] includes NADH, NCDH, NTDH, NGDH, FADH₂, CoQH₂, PQH₂, FMNH₂, NADPH, NCDPH, NGDPH, NTDPH, reduced ferredoxin, reduced cytochrome c, reduced thioredoxins, reduced glutaredoxins, H₂O, etc.

The compound represented by formula (1) includes α-ketonic acid.

The α-ketonic acid is selected from one or more of pyruvic acid, oxaloacetic acid, and α-ketoglutaric acid.

The compound represented by formula (2) includes α-amino acid.

The α-amino acid includes α-alanine, aspartic acid, or glutamic acid.

The aminotransferase includes one or more of BauA from Pseudomonas aeruginosa, AptA from Acinetobacter pittii, CCNA_03245 from Caulobacter vibrioides, PydD2 from Bacillus megaterium, YhxA from Bacillus cereus, PydD from Ureibacillus massiliensis, and Pyd4 from Saccharomyces kluyveri.

An amino acid sequence of the BauA is set forth in SEQ ID NO: 7;
an amino acid sequence of the AptA is set forth in SEQ ID NO: 8;
an amino acid sequence of the CCNA_03245 is set forth in SEQ ID NO: 9;
an amino acid sequence of the PydD2 is set forth in SEQ ID NO: 10;
an amino acid sequence of the YhxA is set forth in SEQ ID NO: 11;
an amino acid sequence of the PydD is set forth in SEQ ID NO: 71; and
an amino acid sequence of the Pyd4 is set forth in SEQ ID NO: 72.

The oxidoreductase includes one or more of MCR-C from Chloroflexus aurantiacu, StMCR from Sulfurisphaera tokodaii, Msed_0709 from Metallosphaera sedula, PnSCR from Pyrobaculum neutrophilum, and SucD from Clostridium kluyveri.

An amino acid sequence of the MCR-C is set forth in SEQ ID NO: 70;
an amino acid sequence of the StMCR is set forth in SEQ ID NO: 12;
an amino acid sequence of the Msed_0709 is set forth in SEQ ID NO: 13;
an amino acid sequence of the PnSCR is set forth in SEQ ID NO: 14; and
an amino acid sequence of the SucD is set forth in SEQ ID NO: 15.

A preparation method for the MCR-C includes the following steps:
1) constructing a recombinant plasmid pCDF-P1-mcrC based on the amino acid sequence of the MCR-C;
2) transferring the recombinant plasmid pCDF-P1-mcrC into E. coli BL21 DE3 competent cells to obtain positive monoclonal colonies;
3) cultivating the positive monoclonal colonies under induction with an inducer;
4) centrifuging and purifying a culture product in step 3), adding a nondenaturing lysis buffer to resuspend a bacterial cell, adding a lysozyme for even mixing, cooling and sonicating, and lysing bacteria; centrifuging, and collecting a supernatant to prepare a bacterial lysis solution; and
5) carrying out chromatography on the bacterial lysis solution prepared in step 4) to prepare a purified MCR-C protein sample.

As another implementation of the present invention, the preparation method for the MCR-C includes the following steps:
1) transferring the recombinant plasmid pCDF-P1-mcrC into E. coli BL21 (DE3) competent cells to obtain positive monoclonal colonies;
2) taking the positive monoclonal colonies to be inoculated into a liquid LB medium containing spectinomycin, and when culturing same until OD₅₅₀=0.5 to 0.8, adding an inducer for induction culture until a final concentration is 0.1 to 0.2 mM;
3) centrifuging a culture product in step 2), collecting an induced bacterial cell, carrying out protein purification with a protein purification kit, adding a nondenaturing lysis buffer to resuspend the bacterial cell, adding a lysozyme for even mixing, cooling and sonicating, and lysing bacteria until a bacterial solution is semi-transparent; centrifuging, and collecting a supernatant to prepare a bacterial lysis solution; and
4) carrying out chromatography on the bacterial lysis solution prepared in step 3) to prepare a purified MCR-C protein sample.

As yet another implementation of the present invention, the preparation method for the MCR-C includes the following steps:
1) transferring the recombinant plasmid pCDF-P1-mcrC into E. coli BL21 (DE3) competent cells to obtain positive monoclonal colonies;
2) taking the positive monoclonal colonies to be inoculated into a liquid LB medium containing spectinomycin, carrying out shaking culture for 8 to 12 hours until OD₅₅₀=2.0 to 2.5 to be used as a seed solution, inoculating the seed solution with an inoculum size of initial OD₅₅₀=0.1 to 0.2 to an LB medium containing spectinomycin, and when culturing same until OD₅₅₀=0.5 to 0.8, adding an inducer isopropyl β-D-1-thiogalactopyranoside (IPTG) for induction culture until a final concentration is 0.1 to 0.2 mM, with a culture condition of shaking culture;
3) centrifuging a culture product in step 2), collecting an induced bacterial cell, carrying out protein purification with a His-tag protein purification kit, adding a nondenaturing lysis buffer to resuspend the bacterial cell, adding a lysozyme for even mixing, cooling and sonicating, and lysing bacteria until a bacterial solution is semi-transparent; centrifuging, and collecting a supernatant to prepare a bacterial lysis solution; and
4) carrying out protein purification with a nickel affinity column: BeyoGold™ His-tag Purification Resin, centrifuging to remove a storage solution, adding a nondenaturing lysis buffer to a gel for even mixing to balance the gel, centrifuging to remove the liquid, adding the bacterial lysis solution prepared in step 3), mixing same well and then packing same into a nickel-column affinity chromatographic column for elution to prepare a purified MCR-C protein sample.

A construction method for the recombinant plasmid pCDF-P1-mcrC includes the following steps:
1) using a pCDF-duet plasmid containing 6×His tag as a template to be amplified by using primers P1-R/P1-F to obtain a pCDF vector backbone fragment, which contains a T7 promoter, an Ori sequence, and a spectinomycin resistance gene Smr and is referred as fragment I, wherein a sequence of P1-R is set forth in SEQ ID NO: 1, a sequence of P1-F is set forth in SEQ ID NO: 2, and a sequence of the fragment I is set forth in SEQ ID NO: 3;
2) using a pMCR-C plasmid as a template to be amplified by using primers MCR-C-pCDF-P1-up/MCR-C-pCDF-P1-down to obtain an MCR-C coding gene, referred as fragment II, wherein a sequence of MCR-C-pCDF-P1-up is set forth in SEQ ID NO: 4, a sequence of MCR-C-pCDF-P1-down is set forth in SEQ ID NO: 5, and a sequence of the fragment II is set forth in SEQ ID NO: 6;
3) removing the templates from the fragment I and the fragment II respectively by using an endonuclease DpnI, and after electrophoresis, cleaning same by using a PCR purification kit, taking the fragment I and the fragment II after template removal, and adding 2× Seamless Cloning Mix for reaction for 20 to 30 minutes; and
4) taking a reaction product in step 3) to be transferred into Trans-T1 competent cells, selecting positive single colonies, and carrying out colony PCR validation, with primers P1-YZ-up (SEQ ID NO: 54)/MCR-C-YZ-down (SEQ ID NO: 55), to obtain a correct plasmid pCDF-P1-mcrC, wherein a sequence of P1-YZ-up is set forth in SEQ ID NO: 54, and a sequence of MCR-C-YZ-down is set forth in SEQ ID NO: 55.

A preparation method for the BauA includes the following steps:
step 1, constructing a recombinant plasmid pCDF-P1-bauA based on the amino acid sequence of the bauA;
step 2, transferring the recombinant plasmid pCDF-P1-bauA into E. coli BL21 DE3 competent cells to obtain positive monoclonal colonies;
step 3, cultivating the positive monoclonal colonies in step 2 under induction with an inducer;
step 4, centrifuging and purifying a culture product in step 3, adding a nondenaturing lysis buffer to resuspend a bacterial cell, adding a lysozyme for even mixing, cooling and sonicating, and lysing bacteria; centrifuging, and collecting a supernatant to prepare a bacterial lysis solution; and
step 5, carrying out chromatography on the bacterial lysis solution prepared in step 4 to prepare a purified BauA protein sample.

### Second Aspect

The present invention provides a recombinant plasmid including BauA and MCR-C, wherein an amino acid sequence of the BauA is set forth in SEQ ID NO: 7; and an amino acid sequence of the MCR-C is set forth in SEQ ID NO: 70.

### Third Aspect

Provided is a method for synthesizing a fatty acid with the recombinant plasmid mentioned above, wherein the recombinant plasmid uses pCDF as a vector and is named pCDF-bauA-mcrC; and the method includes the following steps:

transferring the recombinant plasmid pCDF-bauA-mcrC and a thioesterase expression plasmid into E. coli competent cells to obtain a positive clonal strain, which is subjected to fermentation and cultivation to obtain the fatty acid.

The fatty acid includes octanoic acid, decanoic acid, lauric acid, tetradecanoic acid, or hexadecanoic acid.

A preparation method for the octanoic acid includes the following steps: transferring the recombinant plasmid pCDF-bauA-mcrC and pTrc-TE10 into E. coli competent cells to obtain a positive clonal strain, which is subjected to fermentation and cultivation to obtain the octanoic acid.

A construction method for the recombinant plasmid pCDF-bauA-mcrC includes the following steps:
Step 1, using a pCDF-P1-bauA plasmid as a template to be amplified by using primers P2-MCRC-R/P2-MCRC-F to obtain a pCDF-P1-bauA vector backbone fragment, which contains a T7 promoter, an Ori sequence, a bauA coding sequence, and a spectinomycin resistance gene Smr and is referred as fragment III, wherein a sequence of P2-MCRC-R is set forth in SEQ ID NO: 25, a sequence of P2-MCRC-F is set forth in SEQ ID NO: 26, and a sequence of the fragment III is set forth in SEQ ID NO: 27;
Step 2, using a pCDF-P1-McrC plasmid as a template to be amplified by using primers MCR-C-pCDF-P2-up/MCR-C-pCDF-P2-down to obtain an McrC coding gene, referred as fragment IV, wherein a sequence of the fragment IV is set forth in SEQ ID NO: 28, a sequence of MCR-C-pCDF-P2-up is set forth in SEQ ID NO: 29, and a sequence of MCR-C-pCDF-P2-down is set forth in SEQ ID NO: 30;
Step 3, removing the templates from the fragment III and the fragment IV respectively by using an endonuclease DpnI; after electrophoresis, cleaning same by using a PCR purification kit; taking the fragment III and the fragment IV after template removal, and adding 2× Seamless Cloning Mix for reaction; and
Step 4, taking a reaction product in step 3 to be transferred into Trans-T1 competent cells, selecting positive single colonies, and carrying out colony PCR validation, with primers P2-YZ-up/MCR-C-YZ-down, to obtain a verification-correct plasmid pCDF-bauA-mcrC, wherein a sequence of MCR-C-YZ-down is set forth in SEQ ID NO: 55, and a sequence of P2-YZ-up is set forth in SEQ ID NO: 56.

The recombinant plasmid pCDF-P1-mcrC is constructed according to the amino acid sequence of mcrC, and the amino acid sequence of mcrC is set forth in SEQ ID NO: 70; the recombinant plasmid pCDF-P1-bauA is constructed according to the amino acid sequence of bauA, and the amino acid sequence of bauA is set forth in SEQ ID NO: 7.

### Fourth Aspect

Provided is a method for synthesizing a polyketide with the recombinant plasmid mentioned above, wherein the recombinant plasmid uses pCDF as a vector and is named pCDF-bauA-mcrC; and the method includes the following steps: transferring the recombinant plasmid pCDF-bauA-mcrC and a polyketide synthase coding gene into E. coli competent cells to obtain a positive clonal strain, which is subjected to fermentation and cultivation to obtain the polyketide.

The polyketide includes phloroglucinol, flaviolin, and pentadecaheptaene.

A synthesis method for the phloroglucinol includes the following steps: transferring plasmids pCDF-bauA-mcrC and pCum-phlD into E. coli competent cells to obtain a positive clonal strain, which is subjected to fermentation and cultivation to obtain the phloroglucinol.

A synthesis method for the flaviolin includes the following steps: transferring plasmids pCDF-bauA-mcrC and pCum-rppA into E. coli competent cells to obtain a positive clonal strain, which is subjected to fermentation and cultivation to obtain the flaviolin.

A synthesis method for the pentadecaheptaene includes the following steps: transferring plasmids pCDF-bauA-mcrC and pQL1 into E. coli competent cells to obtain a positive clonal strain, which is subjected to fermentation and cultivation to obtain the pentadecaheptaene.

A construction method for the pCDF-bauA-mcrC is the same as Third aspect mentioned above.

### Fifth Aspect

Provided is a method for synthesizing natamycin or spinosad with the recombinant plasmid, wherein the recombinant plasmid uses SET152 as a vector to express BauA and MCR-C respectively by using strong promoters KasOP and SPL42, and is named SET152-KasOP-bauA-SPL42-mcrC;
a synthesis method for the natamycin includes the following steps:
transferring the recombinant plasmid SET152-KasOP-bauA-SPL42-mcrC into Streptomyces gilvosporeus to obtain a positive clonal strain, which is subjected to fermentation and cultivation to obtain the natamycin; and
a synthesis method for the spinosad includes the following steps:
transferring the recombinant plasmid SET152-KasOP-bauA-SPL42-mcrC into Saccharopolyspora spinosa to obtain a positive clonal strain, which is subjected to fermentation and cultivation to obtain the spinosad.

A construction method for the plasmid pSET152-KasOP-bauA-SPL42-mcrC includes the following steps:
1) cleaving the plasmid pSET152 with XbaI and BamHI, and after agarose gel electrophoresis, recovering a pSET152 backbone fragment 5709 bp, referred as fragment F (SEQ ID NO: 41);
2) using a pLH10 plasmid as a template to be amplified by using primers KasOP-F/R to obtain a KasOP promoter fragment, referred as fragment G (SEQ ID NO: 42); and using a pLH2 plasmid as a template to be amplified by using primers SPL42-F/R to obtain an SPL42 promoter fragment, referred as fragment H (SEQ ID NO: 43), wherein a sequence of KasOP-F is set forth in SEQ ID NO: 46; a sequence of KasOP-R is set forth in SEQ ID NO: 47; a sequence of SPL42-F is set forth in SEQ ID NO: 48; a sequence of SPL42-R is set forth in SEQ ID NO: 49;
3) using a pCDF-bauA-mcrC plasmid as a template to be amplified by using primers BauA-F/R to obtain a BauA gene fragment, referred as fragment J (SEQ ID NO: 44); and amplifying same by using primers McrC-F/R to obtain an McrC gene fragment, referred as fragment K (SEQ ID NO: 45), wherein a sequence of BauA-F is set forth in SEQ ID NO: 50; a sequence of BauA-R is set forth in SEQ ID NO: 51; a sequence of McrC-F is set forth in SEQ ID NO: 52; a sequence of McrC-R is set forth in SEQ ID NO: 53;
4) by using KasOP-F/BauA-R as primers, and using the fragment H and fragment M as templates, linking the fragment H and the fragment M with OE-PCR to be referred as fragment P; and by using SPL42-F/McrC-R as primers, and using the fragment K and fragment N as templates, linking the fragment K and the fragment N with OE-PCR to be referred as fragment Q;
5) taking an appropriate amount of fragment G, fragment P, and fragment Q, and adding 2× Seamless Cloning Mix for reaction; and
6) taking a reaction product in step 5), adding Trans-T1 competent cells to obtain positive single colonies, and carrying out colony PCR validation, with primers BauA-F/R and McrC-F/R, to obtain a verification-correct plasmid pSET 152-KasOP-bauA-SPL42-mcrC.

Compared with the prior art, the present invention has the following beneficial effects:
according to the present invention, by means of heterologous expression of an aminotransferase and a malonyl-CoA reductase, an artificial synthesis pathway for synthesizing malonyl-CoA by using β-alanine (β-ala) as a precursor is constructed as follows: firstly, under catalysis of a transaminase, β-ala transfers amino groups to α-ketonic acid (such as pyruvic acid, oxaloacetic acid, or α-ketoglutaric acid), to form an intermediate product 3-oxopropanoate and a corresponding amino acid; the 3-oxopropanoate generates malonyl-CoA under the action of the malonyl-CoA reductase. This pathway addresses the defects of the natural malonyl-CoA synthesis pathway, such as low carbon utilization, consumption of energy substance ATP, release of greenhouse gas CO₂, and strict regulation of pathway key enzymes, a pyruvate dehydrogenase (PDH) and an acetyl-CoA carboxylase (ACC), thereby achieving the purpose to improve the yield of flaviolin, octanoic acid, phloroglucinol, pentadecaheptaene, natamycin, and spinosad using malonyl-CoA as a precursor. After introducing the artificial malonyl-CoA synthesis pathway, the tolerance of cells to organic acids, osmotic pressure, and cytotoxic substances is improved.

### BRIEF DESCRIPTION OF THE DRAWINGS

By reading the detailed description of non-limiting embodiments with reference to the following drawings, the other features, objectives, and advantages of the present invention will become more apparent:

FIGS. 1A-1C show research status drawings of synthesis pathways for malonyl-CoA and derivatives thereof in BACKGROUND, wherein FIG. 1A is a compound using malonyl-CoA as a precursor; FIG. 1B is a PDH-ACC pathway for synthesizing malonyl-CoA; FIG. 1C is an MCS pathway for synthesizing malonyl-CoA;

FIG. 2 shows a schematic drawing of an NCM pathway according to the present invention;

FIG. 3 shows a transaminase and a reductase after heterologous expression and purification in E. coli, wherein M: Standard protein reference; 1: AptA; 2: BauA; 3: CCNA_03245; 4: PydD2; 5: YhxA; 6: PydD; 7: Pyd4; 8: StMCR; 9: Msed_0709; 10: PnSCR; 11: SucD; 12: MCR-C;

FIGS. 4A-4B show in-vitro activity tests of the transaminase and the reductase, with the transaminase in FIG. 4A and the reductase in FIG. 4B;

FIGS. 5A-5B show schematic drawings of synthesis of 2[H](NADPH) with the transaminase using α-ketoglutaric acid (FIG. 5A) and oxaloacetic acid (FIG. 5B) as substrates;

FIGS. 6A-6C show effects of inhibitors for natural synthesis pathways on an artificial synthesis pathway of NCM, wherein FIG. 6A: The effect of adding different concentrations of ATP on the NCM pathway (the lower the ADP/ATP ratio, the higher the content of ATP); FIG. 6B: The effect of adding different concentrations of acetyl-CoA on the NCM pathway; FIG. 6C: The effect of adding different concentrations of long-chain fatty acyl-CoA (palmitoyl-CoA) on the NCM pathway;

FIGS. 7A-7B show synthesis of malonyl-CoA using the NCM pathway instead of the PDH-ACC pathway, wherein FIG. 7A: CRISPRi technical schematic drawing; FIG. 7B: The growth curve of accBC gene after being suppressed by CRISPRi technology;

FIGS. 8A-8B show that the NCM pathway enhances the ability of a strain to synthesize a fatty acid (octanoic acid, C8), with a schematic drawing of a synthesis pathway of octanoic acid (FIG. 8A), and a histogram of a yield of octanoic acid (FIG. 8B);

FIGS. 9A-9B show that the NCM pathway enhances the ability of a strain to synthesize phloroglucinol, with a schematic drawing of a synthesis pathway of phloroglucinol (FIG. 9A), and a histogram of a yield of phloroglucinol (FIG. 9B);

FIGS. 10A-10B show that the NCM pathway enhances the ability of a strain to synthesize flaviolin, with a schematic drawing of a synthesis pathway of flaviolin (FIG. 10A), and a histogram of a yield of flaviolin (FIG. 10B);

FIGS. 11A-11B show that the NCM pathway enhances the ability of a strain to synthesize pentadecaheptaene, with a schematic drawing of a synthesis pathway of pentadecaheptaene (FIG. 11A), and a histogram of a yield of pentadecaheptaene (FIG. 11B);

FIGS. 12A-12B show that the NCM pathway enhances the ability of a strain to synthesize natamycin, with a schematic drawing of a synthesis pathway of natamycin (FIG. 12A), and a histogram of a yield of natamycin (FIG. 12B);

FIGS. 13A-13C show that the NCM pathway enhances the ability of a strain to synthesize spinosad, with a schematic drawing of a synthesis pathway of spinosad (FIG. 13A), and a histogram of a yield of spinosad (FIGS. 13B-13C);

FIG. 14 shows that the NCM pathway enhances synthesis of a strain NADPH; and

FIGS. 15A-15L show that the NCM pathway enhances tolerance of cells to various growth inhibitions and adverse growth environments.

### DETAILED DESCRIPTION OF THE EMBODIMENTS

The present invention is illustrated in detail below in combination with specific embodiments. The following embodiments will assist those skilled in the art in further understanding the present invention, but do not limit it in any form. It should be pointed out that for those of ordinary skill in the art, without departing from the concept of the present invention, several improvements and modifications can also be made. These are within the protection scope of the present invention.

Experimental methods used in the following embodiments are all conventional methods unless otherwise specified.

In the following embodiments:

His-tag Protein Purification Kit: Beyotime Biotechnology Inc. (His-tag Protein Purification Kit), commonly known as nickel column kit.

TABLE 1
Primers used in the present invention

| | Sequence | |
|---|---|---|
| Name | number | Sequence |
| P1-R | SEQ ID NO: 1 | CGGATCCTGGCTGTGGTG |
| P1-F | SEQ ID NO: 2 | AATTCGAGCTCGGCGCG |
| MCR-C-pCDF-P1- | SEQ ID NO: 4 | CACCACAGCCAGGATCCGAGCGCCACCAC |
| up | | CGGCGC |
| MCR-C-pCDF-P1- | SEQ ID NO: 5 | CGCGCCGAGCTCGAATTTTACACGGTAAT |
| down | | CGCCCGTC |
| P1-YZ-up | SEQ ID NO: 54 | GGATCTCGACGCTCTCCCT |
| MCR-C-YZ-down | SEQ ID NO: 55 | CAGGGTACGTTCAACAAGAT |
| P2-MCRC-R | SEQ ID NO: 25 | GAGATCTGCCATATGTATATCTCCTTC |
| P2-MCRC-F | SEQ ID NO: 26 | AATTGGATATCGGCCGGC |
| MCR-C-pCDF-P2- | SEQ ID NO: 29 | ATATACATATGGCAGATCTCAGCGCCACC |
| up | | ACCGGCGC |
| MCR-C-pCDF-P2- | SEQ ID NO: 30 | GCCGGCCGATATCCAATTTTACACGGTAA |
| down | | TCGCCCGTC |
| P2-YZ-up | SEQ ID NO: 56 | TTGTACACGGCCGCATAATC |
| rppA-Cum-up | SEQ ID NO: 39 | AGAGATTTAAATTCTTTTAAGGAGTAAGT |
| | | ATGGCGACCCTGTGCCGT |
| rppA-Cum-down | SEQ ID NO: 40 | TTGATGCCTGGCTTATCATCATTAACCGCT |
| | | CAGCGCAACACCCG |
| pCF-F | SEQ ID NO: 20 | GGTACCCTCGAGTCTGGTAA |
| pCF-R | SEQ ID NO: 19 | TTACCAGACTCGAGGGTACCTAATTTCGA |
| | | TTATGCGGCCG |
| pecg-accB-F | SEQ ID NO: 23 | CCACTGATTTCCGCTGCTGCGTTTTAGAG |
| | | CTAGAAATAGCAAG |
| pecg-accB-R | SEQ ID NO: 24 | GCAGCAGCGGAAATCAGTGGGCAACCAT |
| | | TATCACCGC |
| Cum-YZ-up | SEQ ID NO: 57 | AAGCAGAGGACTAAGCAGAGA |
| Cum-YZ-down | SEQ ID NO: 58 | CTGATTACCGCCTTTGAGTGA |
| pCum-R | SEQ ID NO: 33 | ACTTACTCCTTAAAAGAATTTAAATCTCT |
| | | AGTAAAT |
| pCum-F | SEQ ID NO: 34 | TGATGATAAGCCAGGCATCAA |
| phID-up | SEQ ID NO: 36 | AAATTCTTTTAAGGAGTAAGTATGAGCAC |
| | | CCTGTGCCTG |
| phID-down | SEQ ID NO: 37 | TTGATGCCTGGCTTATCATCATTACGCGG |
| | | TCCATTCGCC |
| KasOP-F | SEQ ID NO: 46 | GGGCTGCAGGTCGACTCTAGAGGAACGAT |
| | | CGTTGGCTGTG |
| KasOP-R | SEQ ID NO: 47 | GGCTGATTCATATGGCGTATCCCCTTTCA |
| | | G |
| SPL42-F | SEQ ID NO: 48 | CATTGCGTAATATCTGTTCACATTCGAAC |
| SPL42-R | SEQ ID NO: 49 | GATCTGCCATCATATGATGGACACTCCTT |
| | | AC |
| BauA-F | SEQ ID NO: 50 | GATACGCCATATGAATCAGCCGCTGAAC |
| BauA-R | SEQ ID NO: 51 | GTGAACAGATATTACGCAATGCCGTTCAG |
| MCRC-F | SEQ ID NO: 52 | CCATCATATGATGGCAGATCTCAGCGCCA |
| | | C |
| MCRC-R | SEQ ID NO: 53 | TATCGCGCGCGGCCGCGGATCCTTACACG |
| | | GTAATCGCCCGTC |
| Apr-F | SEQ ID NO: 59 | GACGACGAGCCGTTCGATC |
| Apr-R | SEQ ID NO: 60 | CTTCTCCTTGAGCCACCTG |

TABLE 2
Plasmids used in the present invention

| Name | Description | Reference |
|---|---|---|
| pCDF-duet | Double T7 promoters, CloDF13 | Invitrogen |
| | ori, Sm^{r} | |
| pMCR-C | lacI P_{T7} His6-mcr550-1219 N940V | C Liu et |
| | K1106WS1114R, pBR322 ori, Amp^{R} | al., 2016 |
| pEcCas9 | PrhaB sgRNA, P_{araBAD} Cas9, sacB, | Li Q I et |
| | pSC101 ori, Kan^{r} | al., 2021 |
| pQL1 | P_{T7}: N-terminal his6-tag | Q Liu et |
| | codon-optimized sgcE, and P_{Lac-1}: | al., 2015 |
| | N-terminal his6-tag codon-optimized | |
| | sgcE10, Kan^{r} | |
| pEcgRNA | Pⱼ₂₃₁₁₉ N20 gRNA scaffold, ccdB, | Li Q I et |
| | Sm^{r} | al., 2021 |
| pCum | p4201_CymR-pT5-CuO-T3target- | Hou J et |
| | RiboJ-sfGFP, pSC101 ori, Kan^{r} | al., 2020 |
| pCF-tesA | P_{trc}-dCas9, P_{T7}-tesA, p15A ori, Cmr | L Fang, et |
| | | al., 2020 |
| Sg-0-RNA | ColE ori, Amp, pCI-sgRNA-scaffold | L Fang et |
| | | al., 2020 |
| pCDF-P1-mcrC | pCDF-duet carrying mcrC | The present |
| | | invention |
| pCDF-P1-aptA | pCDF-duet carrying aptA | The present |
| | | invention |
| pCDF-P1- | pCDF-duet carrying CCNA_03245 | The present |
| CCNA_03245 | | invention |
| pCDF-P1-pydD2 | pCDF-duet carrying pydD2 | The present |
| | | invention |
| pCDF-P1-yhxA | pCDF-duet carrying yhxA | The present |
| | | invention |
| pCDF-P1-StMcr | pCDF-duet carrying StMcr | The present |
| | | invention |
| pCDF-P1- | pCDF-duet carrying Msed_0709 | The present |
| Msed_0709 | | invention |
| pCDF-P1-scr | pCDF-duet carrying scr | The present |
| | | invention |
| pCDF-P1-SucD | pCDF-duet carrying SucD | The present |
| | | invention |
| pCDF-P1-bauA | pCDF-duet carrying bauA | The present |
| | | invention |
| pCDF-pydD | pCDF-duet carrying pydD | The present |
| | | invention |
| pCDF-pyd4 | pCDF-duet carrying pyd4 | The present |
| | | invention |
| pCDF-bauA-mcrC | pCDF-duet carrying bauA and mcrC | The present |
| | by two promoter | invention |
| pCum-rppA | p4201_CymR-pT5-CuO-rppA, | The present |
| | pSC101 ori, Kan^{r} | invention |
| pCum-phlD | p4201_CymR-pT5-CuO-phlD, | The present |
| | pSC101 ori, Kan^{r} | invention |
| pTrc-TE10 | pTrc-TE10, pSC101 ori, Amp^{r} | Z Tan, et |
| | | al, 2016 |
| pSET152 | aac(3)IV, lacZ, reppMB1*, attΦC31, | M. Bierman |
| | oriT | et al., 1992 |
| pLH10 | pSET152 derivative with insertion of | Q Liu et |
| | the synthetic promoter kasOp | al., 2016 |
| | regulated gusA | |
| pLH2 | pSET152 derivative with insertion of | Q Liu et |
| | the synthetic promoter SPL42 | al., 2016 |
| | regulated gusA | |
| pSET152-KasOP- | pSET152 carrying bauA and mcrC by | The present |
| bauA-SPL42-mcrC | two promoter | invention |

TABLE 3
Strains used in the present invention

| Name | Description | Reference |
|---|---|---|
| E. coli BL21(DE3) | F⁻ dcm ompT hsdS (rB⁻ | Tolo Biotech |
| | mB⁻) gal(DE3) | Co., Ltd. |
| E. coli MG1655 | F⁻ lambda⁻ ilvG rfb-50 | Tolo Biotech |
| (DE3) | rph-1 (DE3) | Co., Ltd. |
| E. coli | dam dcm hsdS/pUB307 | MacNeil, D. J. |
| ET12567/pUB307 | | et al., 1992 |
| Streptomyces | S. gilvosporeus, Natamycin | ATCC ® 13326 ™ |
| gilvosporeus J1-002 | producer strain | |
| Saccharopolyspora | S. spinosa, Spinosad | CN202110670419.8 |
| spinosa WHU1107 | producer strain | CCTCC. NO: M |
| | | 2021307 |
| E. coli | F⁻ mcrA | YouBio |
| ETU12567/ | Δ(mrr-hsdRMS-mcrBC) | |
| pUZ8002 | | |
| DH10B | | |

### Embodiment 1 Design of New Synthesis Pathway of Malonyl-CoA

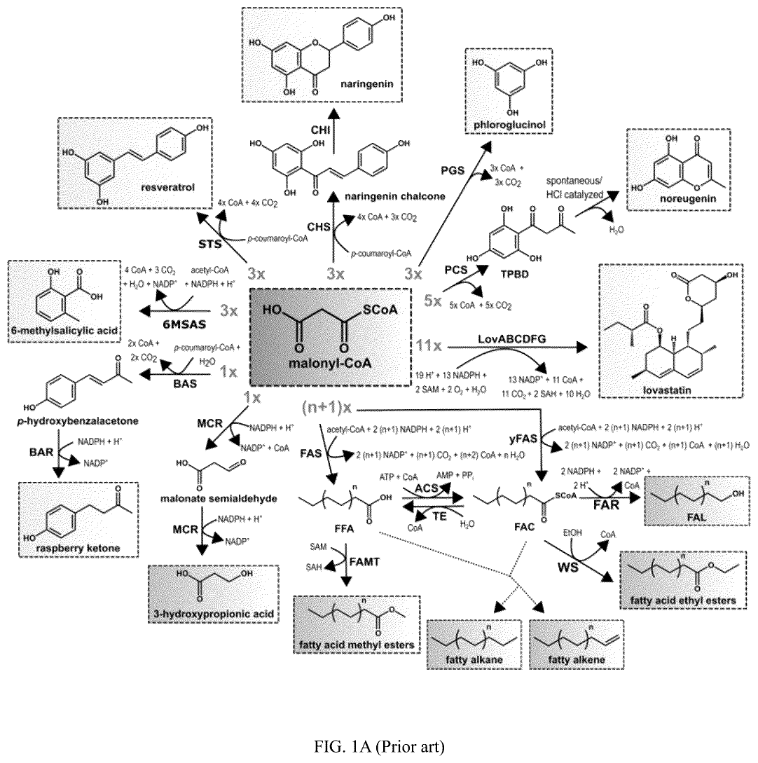

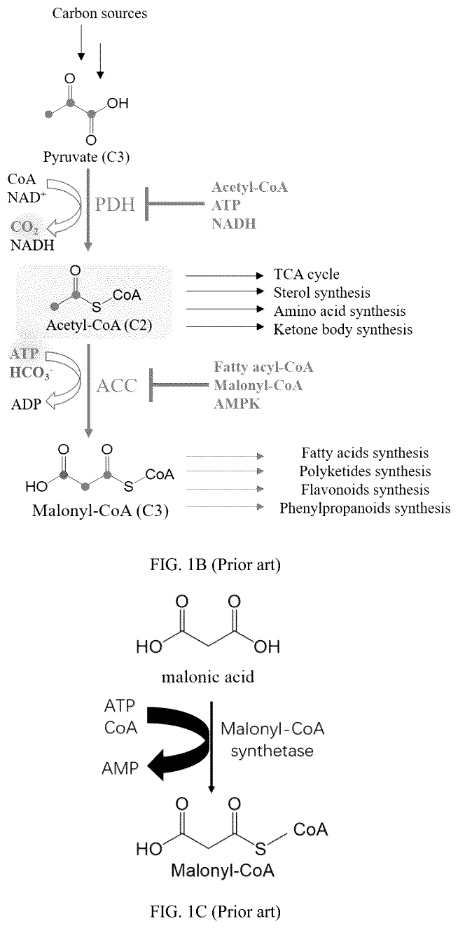

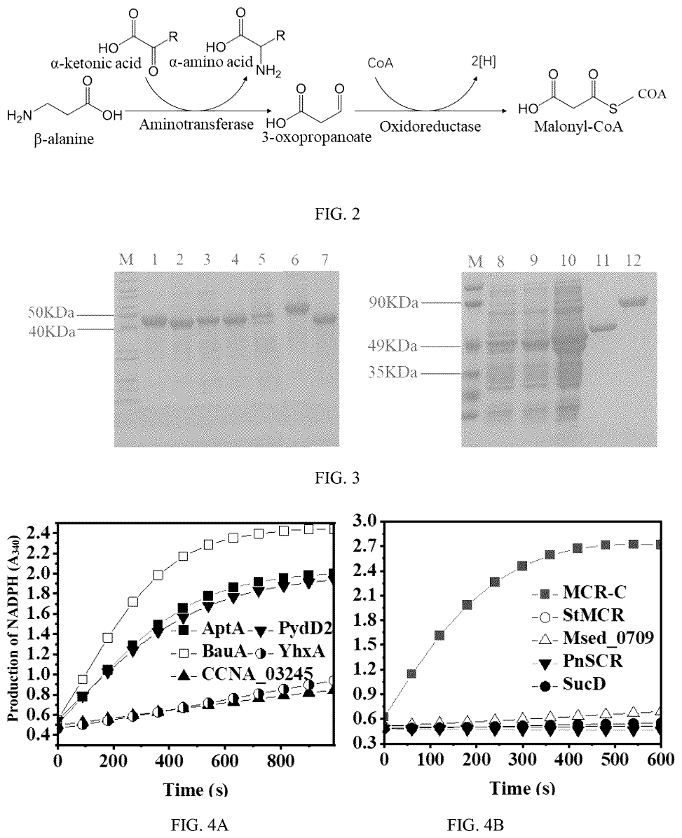

Since pyruvic acid is the most common product of carbon source catabolism, we attempt to introduce a new link molecule between pyruvic acid (C3) and malonyl-CoA (C3). Unlike the natural pathway, C3 (pyruvic acid)-C2 (acetyl-CoA)-C3 (malonyl-CoA) mode, the present invention adopts a new mode of “C3-C3-C3” (FIG. 2), thereby avoiding ineffective reaction of “decarboxylation and carboxylation” (FIG. 1A). From the numerous C3 linked molecules, we select 3-oxopropanoate. Specifically, pyruvic acid and β-alanine first form 3-oxopropanoate and α-alanine under catalysis of transaminases or aminotransferases, and the 3-oxopropanoate then forms malonyl-CoA under catalysis of oxidoreductases (FIG. 2). In this design, due to artificial pathways without CO₂ release, carbon source loss and ATP consumption, the problem of carbon source and energy waste inherent in natural pathways is avoided (FIG. 1A). In addition, unlike a central metabolite, acetyl-CoA, 3-oxopropanoate is not a central metabolite in the vast majority of living cells, so the artificial pathways do not have the problem of high competition with a cellular background metabolic network. We refer to this design as a non-carboxylative malonyl-CoA (NCM) pathway. Compared to natural synthesis pathways, the advantages exhibited by artificial synthesis are detailed in Table 1.

TABLE 4
Comparison between artificial pathway and natural pathway

| | Natural pathway | Artificial pathway |
|---|---|---|
| Product | Malonyl-CoA | |
| Substrate | Pyruvic acid | |
| Metabolic intermediate | Acetyl-CoA | 3-oxopropanoate |
| Reaction rate | 0.25-23.7 | 2.0 × 10³ |
| (nmol/min/mg) | | |
| Number of subunits of | 7 | 2 |
| required enzymes ^{a} | | |
| Relative molecular mass | 5000-10000 | 350 |
| (kDa) of enzymes ^{b} | | |
| Number of required | 7 | 3 |
| cofactors ^{c} | | |
| Number of generated ATPs ^{d} | −1 | 0 |
| Oxidation-reduction | +1 NADH | +1 2[H] |
| product ^{d} | | |
| Carbon dioxide generated | +1 | 0 |
| by per molecular product ^{d} | | |
| Carbon utilization rate ^{e} | 67% | 100% |
| Number of reaction steps | 2 | 2 |
| Types of reactions involved | Decarboxylation | Transamination |
| | reaction, | reaction, |
| | Carboxylation | Oxidation |
| | reaction | reaction |
| ΔᵣG′°(kcal/mol)^{f} | −14.3 | −8.9 |
| Degree of competition with | +++ | + |
| other metabolic pathways ^{g} | | |
| Degree of regulation ^{h} | +++ | + |
| Operability ⁱ | + | +++ |

Note:
^{a} Pyruvate dehydrogenase contains three subunits (E1, E2, and E3), acetyl-CoA carboxylase contains four subunits (AccA, AccB, AccC and AccD), while NCM pathway enzymes, BauA and MCR-C are both single subunits
^{b} The molecular weight of pyruvate dehydrogenase is approximately 4,600 kDa in Gram-negative bacteria, while it is approximately 8,000 to 10,000 kDa in Gram-positive bacteria and fungi. In the NCM pathway, the molecular weight of BauA is approximately 200 kDa, and the molecular weight of MCR-C is approximately 150 kDa
^{c} The natural malonyl-CoA synthesis pathway requires seven cofactors, namely thiamine pyrophosphate, FAD, CoA, NAD⁺, lipoic acid, biotin, and ATP. However, the NCM pathway only requires three cofactors, namely pyridoxal phosphate, NADP⁺, and CoA.
^{d} + represents generation, and − represents consumption.
^{e} The carbon utilization rate is the ratio of the number of carbon atoms in the acyl backbone of the produce malonyl-CoA derived from the substrate pyruvic acid to the number of carbon atoms of the acyl backbone of the malonyl-CoA.
^{f}Standard ΔᵣG′° is calculated by the difference in Gibbs free energy between products and substrates in biochemical reaction
^{g} The natural pathway competes with various metabolic pathways, such as TCA cycle, and amino acid and steroid synthesis pathways. On the contrary, the artificial pathway does not compete with important cellular metabolic pathways. The competition intensity is divided into three levels: high (+++), medium (++), and low (+).
^{h} The natural pathway is closely regulated by various factors, such as allosteric inhibition of key enzymes, product feedback inhibition, and energy substance ATP inhibition. However, these inhibitors have no significant inhibitory effect on the artificial pathway. The degree of being closely regulated is divided into three levels: high (+++), medium (++), and low (+).
ⁱ Implementation operability is estimated based on factors such as the availability of enzymes, the number of enzymes required, the ability to express enzymes, and the complexity of enzymes. Operability is divided into three levels: difficult (+++), moderate (++), and easy (+).

### Embodiment 2 Exploitation and Testing of Candidate Enzymes of New Synthesis Pathway for Malonyl-CoA

Since the NCM pathway consists of transaminases and oxidoreductases, we attempted to screen candidate enzymes from different sources for separate testing and combined utilization. Specifically:

In the exploitation of transaminases, we selected BauA (from Pseudomonas aeruginosa), AptA (from Acinetobacter pittii), CCNA_03245 (from Caulobacter vibrioides), PydD2 (from Bacillus megaterium), YhxA (from Bacillus cereus), PydD (from Ureibacillus massiliensis), and Pyd4 (from Saccharomyces kluyveri);

In the exploitation of oxidoreductases, we selected MCR-C (from Chloroflexus aurantiacu), StMCR (from Sulfurisphaera tokodaii), Msed_0709 (from Metallosphaera sedula), PnSCR (from Pyrobaculum neutrophilum), and SucD (from Clostridium kluyveri);

The following were the construction of candidate enzyme expression plasmids, purification of candidate enzymes, in-vitro reactions, and other operational procedures:

### 1. Construction of Candidate Enzyme Expression Plasmids

1.1. Construction of Plasmid pCDF-P1-mcrC

The plasmid was constructed based on an amino acid sequence of MCR-C, wherein the amino acid sequence of the MCR-C was set forth in SEQ ID NO: 70.

1) A pCDF-duet plasmid containing 6×His tag was used as a template to be amplified by using primers P1-R/P1-F to obtain a pCDF vector backbone fragment, with a fragment size of 3781 bp, containing a T7 promoter, an Ori sequence, and a spectinomycin resistance gene Smr (Accession No. UKC63634.1), and referred as fragment I, wherein a sequence of P1-R was set forth in SEQ ID NO: 1, a sequence of P1-F was set forth in SEQ ID NO: 2, and a sequence of the fragment I was set forth in SEQ ID NO: 3;

An amplification system was as follows: 25 μL of Phanta 2× Buffer, 1 μL of dNTP (10 mM for each dNTP), 20 ng of DNA template, 2 μL of each primer (10 M), 1 μL of Phanta Max Super-Fidelity DNA polymerase, and 18 μL of distilled water, with a total volume of 50 μL.

Amplification conditions were as follows: pre-denaturation at 95° C. for 3 minutes (1 cycle); denaturation at 95° C. for 15 seconds, annealing at 56° C. for 15 seconds, extension at 72° C. for 120 seconds (30 cycles); extension at 72° C. for 5 minutes (1 cycle).

2) A pMCR-C plasmid was used as a template to be amplified by using primers MCR-C-pCDF-P1-up/MCR-C-pCDF-P1-down to obtain an MCR-C coding gene, with a gene fragment size of 2048 bp, referred as fragment II, wherein a sequence of MCR-C-pCDF-P1-up was set forth in SEQ ID NO: 4, a sequence of MCR-C-pCDF-P1-down was set forth in SEQ ID NO: 5, and a sequence of the fragment II was set forth in SEQ ID NO: 6;

An amplification system was as follows: 25 μL of Phanta 2× Buffer, 1 μL of dNTP (10 mM for each dNTP), 20 ng of DNA template, 2 μL of each primer (10 M), 1 μL of Phanta Max Super-Fidelity DNA polymerase, and 18 μL of distilled water, with a total volume of 50 μL.

Amplification conditions were as follows: pre-denaturation at 95° C. for 3 minutes (1 cycle); denaturation at 95° C. for 15 seconds, annealing at 56° C. for 15 seconds, extension at 72° C. for 60 seconds (30 cycles); extension at 72° C. for 5 minutes (1 cycle).

3) The templates were removed from the fragment I obtained in the first step and the fragment II obtained in the second step respectively by using an endonuclease DpnI (New England Biolabs (NEB) Inc.), with cleavage at 37° C. for 30 minutes, and after agarose electrophoresis, the fragment I and fragment II were cleaned by using a PCR purification kit (FastPure Gel DNA Extraction Mini Kit, purchased from Vazyme Biotech Co., Ltd.); an appropriate amount of the fragment I and an appropriate amount of the fragment II (V_{I}+V_{II}=5 μL, n_{I}/n_{II}=1/3) were taken, and 5 μL of 2× Seamless Cloning Mix (Beyotime Biotechnology Inc.) was added for reaction at 50° C. for 30 minutes.

4) 2 μL of a reaction product in step 3) was taken to be added into 50 μL of Trans-T1 competent cells (purchased from Beijing TransGen Biotech Co., Ltd.), and ice bath was carried out for 30 minutes. Heat shock was carried out at 42° C. for 30 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL). After culture for 12 hours, 5 positive single colonies were selected for colony PCR validation, with primers P1-YZ-up (SEQ ID NO: 54)/MCR-C-YZ-down (SEQ ID NO: 55). A clone with a PCR fragment of 488 bp validated as a positive clone was obtained. Two positive clones were selected to extract plasmids for sequencing analysis. By means of comparing sequencing results, a correct plasmid pCDF-P1-mcrC was obtained.

1.2. The construction of plasmids pCDF-P1-bauA, pCDF-P1-aptA, pCDF-P1-CCNA_03245, pCDF-P1-pydD2, pCDF-P1-yhxA, pCDF-P1-StMcr, pCDF-P1-Msed_0709, pCDF-P1-PnScr, and pCDF-P1-sucD was completed by Genewiz Inc. based on corresponding amino acids (the construction method was the same as the plasmid pCDF-P1-mcrC). The sequences of the amino acids involved were as follows:

| | Amino acid sequence |
|---|---|
| BauA | SEQ ID NO: 7 |
| AptA | SEQ ID NO: 8 |
| CCNA_03245 | SEQ ID NO: 9 |
| PydD2 | SEQ ID NO: 10 |
| YhxA | SEQ ID NO: 11 |
| PydD | SEQ ID NO: 71 |
| Pyd4 | SEQ ID NO: 72 |
| StMcr | SEQ ID NO: 12 |
| Msed_0709 | SEQ ID NO: 13 |
| PnScr | SEQ ID NO: 14 |
| SucD | SEQ ID NO: 15 |

### 2. Purification of Candidate Enzymes

### 2.1. Purification of MCR-C

First step: 50 ng of the plasmid pCDF-P1-mcrC was taken to be added into 50 μL of E. coli BL21 (DE3) competent cells, and ice bath was carried out for 30 minutes. Heat shock was carried out at 42° C. for 60 seconds, and the same was immediately placed on ice for 2 minutes. 500 μL of an SOC medium was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL). After overnight culture, positive colonies were obtained.

Second step: 5 positive monoclonal colonies were taken to be inoculated into 5 mL of a liquid LB seed medium containing spectinomycin (a final concentration of 50 μg/mL), and culture was carried out at 220 rpm and 37° C. for 8 hours until OD₅₅₀=2.0. A seed solution with an inoculum size of initial OD₅₅₀=0.1 was inoculated into 50 mL of a liquid LB medium (containing spectinomycin with a final concentration of 50 μg/mL). When culture was carried out at 220 rpm and 37° C. until OD₅₅₀=0.5 to 0.8, an inducer, isopropyl β-D-1-thiogalactopyranoside (IPTG), was added until a final concentration was 0.1 mM, and culture continued at 220 rpm and 37° C. for 4 to 5 hours.

Third step: Centrifugation was carried out at 6000 g for 10 minutes to collect the induced bacterial cell. Next, protein purification was carried out with His-tag protein purification kit (purchased from Beyotime Biotechnology Inc.). A lysis solution was added at a ratio of adding 4 mL of a nondenaturing lysis buffer to each gram of bacterial precipitation wet weight, to fully resuspend the bacterial cell. A lysozyme was added until a final concentration was 1 mg/mL, even mixing was carried out, and the same was placed on ice for 30 minutes. Under cooling of an ice-water mixture, bacteria were subjected to ultrasonic lysis, with ultrasonic power of 150 to 250 W, with ultrasonic processing for 2 seconds each time, with an interval of 2 seconds, and with the total duration of ultrasonic processing of 5 to 10 minutes (until the bacterial solution was semi-transparent); centrifugation was carried out at 10000 g at 4° C. for 20 to 30 minutes to collect a supernatant, so as to prepare a bacterial lysis solution, which was placed on ice.

Fourth step: protein purification was carried out with a nickel affinity column, specifically, 1 mL of evenly-mixed 50% BeyoGold™ His-tag Purification Resin was taken to be centrifuged (1000 g×10 s) at 4° C. so as to remove a storage solution. 0.5 mL of the nondenaturing lysis buffer was added to a gel to be mixed well to balance the gel, centrifugation was carried out (1000 g×10 s) at 4° C. to remove the liquid, and the balance was repeated 1 to 2 times to remove the liquid. Approximately 4 mL of the bacterial lysis solution prepared in the third step was added therein, and slow shaking was carried out at 4° C. for 60 minutes. Afterwards, a mixture of the lysis solution and BeyoGold™ His-tag Purification Resin was packed into a nickel-column affinity chromatographic column. A lid at the bottom of the purification column was opened, and a liquid inside the column was allowed to flow out under gravity for column washing 5 times, with adding 0.5 to 1 mL of a nondenaturing washing solution each time. A target protein was eluted 6 to 10 times, with 0.5 mL of a nondenaturing elution solution each time. The eluents each time were collected separately into different centrifuge tubes. The collected eluents were purified His-tag protein samples.

Fifth step: the above eluents were added to 2 mL ultrafiltration tubes (Sartorius), which were placed in a centrifugal machine to be centrifuged at 4° C. and 13000 r until all the eluents were concentrated to 50 μL. 500 μL of a TEG buffer (50 mM Tris-HCl, 0.5 mM EDTA, 50 mM NaCl, 5% glycerol, pH 7.9) was then added. Centrifugation was carried out at 4° C. and 13000 r until the solution was concentrated to 50 μL. 500 μL of a TEG buffer (50 mM Tris-HCl, 0.5 mM EDTA, 50 mM NaCl, 5% glycerol, pH 7.9) was then added. Centrifugation was carried out at 4° C. and 13000 r until approximately 50 to 100 μL of a protein solution was finally left over in the ultrafiltration tubes. The protein solution was blown and sucked for even mixing with a pipette, and then transferred to pre-cooled EP tubes.

Sixth step: the concentrations of MCR-C proteins obtained by purification were determined with a BCA protein concentration determination kit (purchased from Beyotime Biotechnology Inc.). According to the number of the samples, an appropriate amount of a BCA working solution was prepared at a ratio of adding 50 volumes of BCA reagent A to 1 volume of BCA reagent B (50:1), and mixed thoroughly. 20 μL of a 25 mg/mL protein standard was taken, and 980 μL of a diluent was added, so that a 0.5 mg/mL protein standard could be prepared. The 0.5 mg/mL protein standard solution was diluted to 0.05 (10→90), 0.1 (20→80), 0.2 (40→60), 0.3 (60→40), 0.4 (80→20) mg/mL, respectively. 0 to 0.5 mg/mL standard products were added to 96-well plates in sequence, each with 20 μL of samples added (three groups being parallel). After initial determination in Nano-300, the samples were diluted to a certain multiple, and 20 μL of the samples were added to sample wells of the 96-well plates. 200 μL of a BCA working solution was added to each well, and standing was carried out at 37° C. for 30 minutes. The absorbance at a wavelength of 562 nm was determined by a microplate reader. A standard curve was made based on the absorbance at 562 nm and concentration of the standard protein solutions, and the concentrations of proteins purified were calculated based on the standard curve.

The purification methods for other reductases StMCR, Msed_0709, PnSCR, and SucD were the same as those for the MCRC.

### 2.2. Purification of BauA

A preparation method for a BauA protein sample included the following steps:

First step: 50 ng of a plasmid pCDF-P1-bauA was taken to be added into 50 μL of E. coli BL21 (DE3) competent cells, and ice bath was carried out for 30 minutes. Heat shock was carried out at 42° C. for 60 seconds, and the same was immediately placed on ice for 2 minutes. 500 μL of an SOC medium was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL). After overnight culture, positive colonies were obtained.

Second step: 5 positive monoclonal colonies were taken to be inoculated into 5 mL of a liquid LB seed medium containing spectinomycin (a final concentration of 50 μg/mL), and culture was carried out at 220 rpm and 37° C. for 8 hours until OD₅₅₀=2.0. A seed solution with an inoculum size of initial OD₅₅₀=0.1 was inoculated into 50 mL of a liquid LB medium (containing spectinomycin with a final concentration of 50 μg/mL). When culture was carried out at 220 rpm and 37° C. until OD₅₅₀=0.5 to 0.8, an inducer, isopropyl β-D-1-thiogalactopyranoside (IPTG), was added until a final concentration was 0.1 mM, and culture continued at 220 rpm and 37° C. for 4 to 5 hours.

Third step: Centrifugation was carried out at 6000 g for 10 minutes to collect the induced bacterial cell. Next, protein purification was carried out with His-tag protein purification kit (purchased from Beyotime Biotechnology Inc.). A lysis solution was added at a ratio of adding 4 mL of a nondenaturing lysis buffer to each gram of bacterial precipitation wet weight, to fully resuspend the bacterial cell. A lysozyme was added until a final concentration was 1 mg/mL, even mixing was carried out, and the same was placed on ice for 30 minutes. Under cooling of an ice-water mixture, bacteria were subjected to ultrasonic lysis, with ultrasonic power of 150 to 250 W, with ultrasonic processing for 2 seconds each time, with an interval of 2 seconds, and with the total duration of ultrasonic processing of 5 to 10 minutes (until the bacterial solution was semi-transparent); centrifugation was carried out at 10000 g at 4° C. for 20 to 30 minutes to collect a supernatant, so as to prepare a bacterial lysis solution, which was placed on ice.

Fourth step: protein purification was carried out with a nickel affinity column, specifically, 1 mL of evenly-mixed 50% BeyoGold™ His-tag Purification Resin was taken to be centrifuged (1000 g×10 s) at 4° C. so as to remove a storage solution. 0.5 mL of the nondenaturing lysis buffer was added to a gel to be mixed well to balance the gel, centrifugation was carried out (1000 g×10 s) at 4° C. to remove the liquid, and the balance was repeated 1 to 2 times to remove the liquid. Approximately 4 mL of the bacterial lysis solution supernatant was added therein, and slow shaking was carried out at 4° C. for 60 minutes. Afterwards, a mixture of the lysis solution and BeyoGold™ His-tag Purification Resin was packed into a nickel-column affinity chromatographic column. A lid at the bottom of the purification column was opened, and a liquid inside the column was allowed to flow out under gravity for column washing 5 times, with adding 0.5 to 1 mL of a nondenaturing washing solution each time. A target protein was eluted 6 to 10 times, with 0.5 mL of a nondenaturing elution solution each time. The eluents each time were collected separately into different centrifuge tubes. The collected eluents were purified His-tag protein samples.

Fifth step: the above eluents were added to 2 mL ultrafiltration tubes (Sartorius), which were placed in a centrifugal machine to be centrifuged at 4° C. and 13000 r until all the eluents were concentrated to 50 μL. 500 μL of a TEG buffer (50 mM Tris-HCl, 0.5 mM EDTA, 50 mM NaCl, 5% glycerol, pH 7.9) was then added. Centrifugation was carried out at 4° C. and 13000 r until the solution was concentrated to 50 μL. 500 μL of a TEG buffer (50 mM Tris-HCl, 0.5 mM EDTA, 50 mM NaCl, 5% glycerol, pH 7.9) was then added. Centrifugation was carried out at 4° C. and 13000 r until approximately 50 to 100 μL of a protein solution was finally left over in the ultrafiltration tubes. The protein solution was blown and sucked for even mixing with a pipette, and then transferred to pre-cooled EP tubes.

Sixth step: the concentrations of MCR-C proteins obtained by purification were determined with a BCA protein concentration determination kit (purchased from Beyotime Biotechnology Inc.). According to the number of the samples, an appropriate amount of a BCA working solution was prepared at a ratio of adding 50 volumes of BCA reagent A to 1 volume of BCA reagent B (50:1), and mixed thoroughly. 20 μL of a 25 mg/mL protein standard was taken, and 980 μL of a diluent was added, so that a 0.5 mg/mL protein standard could be prepared. The 0.5 mg/mL protein standard solution was diluted to 0.05 (10→90), 0.1 (20→80), 0.2 (40→60), 0.3 (60→40), 0.4 (80→20) mg/mL, respectively. 0 to 0.5 mg/mL standard products were added to 96-well plates in sequence, each with 20 μL of samples added (three groups being parallel). After initial determination in Nano-300, the samples were diluted to a certain multiple, and 20 μL of the samples were added to sample wells of the 96-well plates. 200 μL of a BCA working solution was added to each well, and standing was carried out at 37° C. for 30 minutes. The absorbance at a wavelength of 562 nm was determined by a microplate reader. A standard curve was made based on the absorbance at 562 nm and concentration of the standard protein solutions, and the concentrations of proteins purified were calculated based on the standard curve.

The purification methods for other transaminases AptA, CCNA_03245, PydD2, YhxA, PydD, and Pyd4 were the same as those for the BauA.

The results of SDS-PAGE electrophoresis of transaminases and reductases are shown in FIG. 3.

### Embodiment 3 In-Vitro Reaction (Artificial Synthesis of Malonyl-CoA)

Transaminase activity test using pyruvic acid as a substrate: in-vitro reaction was carried out in a total reaction volume of 200 μL, containing 100 mM Tris buffer (pH 7.8), 2 mM MgCl₂, 3 mM DTT, 5 mM pyruvic acid, 5 mM β-alanine, 2.5 mM NADP⁺, 2.5 mM CoA, 1 M purified transaminase, and 10 M MCR-C enzyme, the reaction mixture was cultured at 37° C., and the increase in 340 nm (NADPH generated by the reaction had an absorption peak at 340 nm) was monitored, where the activity of the transaminases was respectively as follows: BauA (1.60 μmol/min/mg), PydD2 (0.96 μmol/min/mg), AptA (0.90 μmol/min/mg), YhxA (0.15 μmol/min/mg), and CCNA_03245 (0.13 μmol/min/mg). It can be seen that the BauA has the best effect (FIG. 4A).

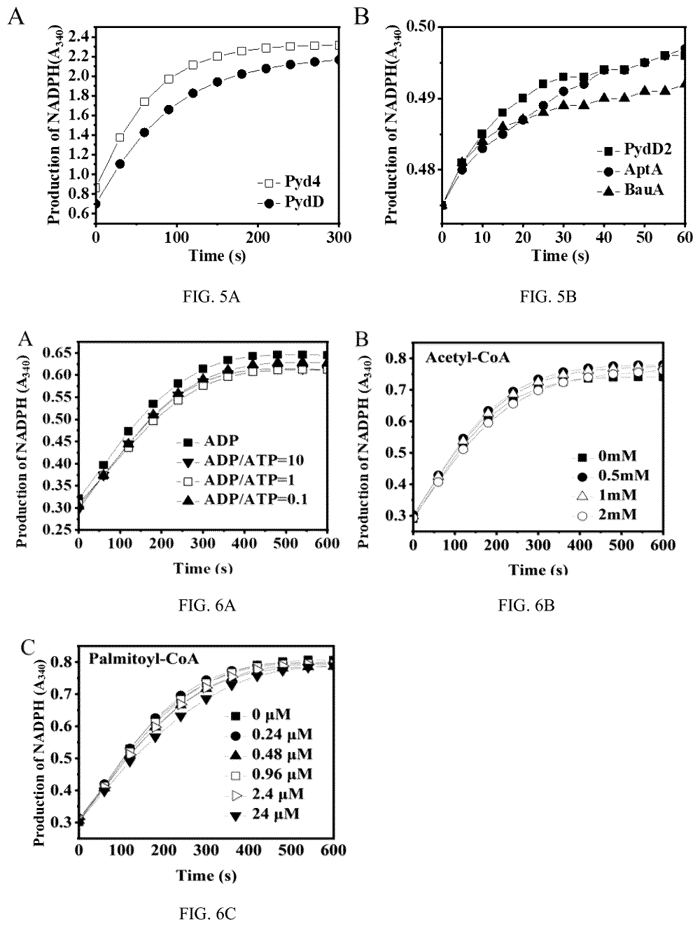

Transaminase activity test using α-ketoglutaric acid as a substrate: in-vitro reaction was carried out in a total reaction volume of 200 μL, containing 100 mM Tris buffer (pH 7.8), 2 mM MgCl₂, 3 mM DTT, 5 mM α-ketoglutaric acid, 5 mM β-alanine, 2.5 mM NADP⁺, 2.5 mM CoA, 10 M purified transaminase, and 10 M MCR-C enzyme, the reaction mixture was cultured at 37° C., and the increase in 340 nm (NADPH generated by the reaction had an absorption peak at 340 nm) was monitored, where the activity of the transaminases was respectively as follows: pydD (0.825 μmol/min/mg), Pyd4 (1.15 μmol/min/mg) (FIG. 5A).

Transaminase activity test using oxaloacetic acid as a substrate: in-vitro reaction was carried out in a total reaction volume of 200 μL, containing 100 mM Tris buffer (pH 7.8), 2 mM MgCl₂, 3 mM DTT, 5 mM oxaloacetic acid, 5 mM β-alanine, 2.5 mM NADP⁺, 2.5 mM CoA, 10 M purified transaminase, and 10 M MCR-C enzyme, the reaction mixture was cultured at 37° C., and the increase in 340 nm (NADPH generated by the reaction had an absorption peak at 340 nm) was monitored, where the activity of the transaminases was respectively as follows: BauA (0.46 μmol/min/mg), ApaT (0.42 μmol/min/mg), PydD2 (0.49 μmol/min/mg) (FIG. 5B).

Oxidoreductase activity test: in-vitro reaction was carried out in a total reaction volume of 200 μL, containing 100 mM Tris buffer (pH 7.8), 2 mM MgCl₂, 3 mM DTT, 5 mM pyruvic acid, 5 mM 3-alanine, 2.5 mM NADP⁺, 2.5 mM CoA, 10 M BauA, and 1 M purified oxidoreductase, the reaction mixture was cultured at 37° C., and the increase in 340 nm was monitored. According to the in-vitro reaction, it can be seen that, both MCR-C and Msed_0709 exhibit the activity of the target enzyme, where the MCR-C has the highest activity (2.0 μmol/min/mg) (FIG. 4B).

### Embodiment 4 the New Synthesis Pathway of Malonyl-CoA Relieves Multiple Feedback Inhibition

Natural PDH-ACC pathways are strictly regulated by many factors, such as high levels of intracellular ATP severely reducing the activity of PDH. When ADP/ATP=1, the activity of PDH is only 10% of that when ADP/ATP=20 (Hansford R G et al., 1976). Acetyl-CoA is also an inhibitor of PDH. When acetyl-CoA reaches 2 mM, the activity of PDH decreases by 70%. Long-chain fatty acyl-CoA, such as palmitoyl-CoA, is an inhibitor of ACC. 0.24 M palmitoyl-CoA can cause an 80% decrease in ACC activity. In the above NCM in-vitro reaction system, ATP and ADP (ATP+ADP=3.75 mM, ADP/ATP was +00, 10, 1, and 0.1, respectively) (FIG. 6A), acetyl-CoA (0, 0.5, 1, and 2 mM) (FIG. 6B), and palmitoyl-CoA (0, 0.24, 0.48, 0.96, 2.4, and 24 M) (FIG. 6C) were added, respectively. In-vitro experimental results demonstrate that these typical inhibitors of natural synthesis pathways have no inhibitory effect on the artificial NCM pathway.

### Embodiment 5 New Synthetic Pathway can Replace Natural Pathways for Synthesis of Intracellular Malonyl-CoA

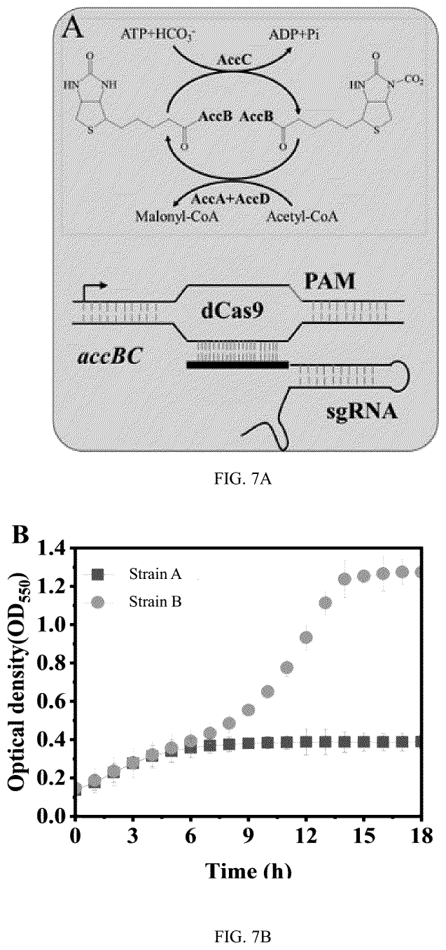

(I) Using CRISPR Interference (CRISPRi) Technology to Inhibit Expression of Key accBC Gene in Natural Synthesis Pathways of Malonyl-CoA in E. coli MG1655 (DE3) (FIG. 7A)
1. Construction of Plasmids pACYC-dCas9 and Sg-accBC
1.1. Construction of Plasmid pACYC-dCas9

First step: a pCF-tesA plasmid was used as a template to be amplified by using primers pCF-R/pCF-F to obtain a pACYC-dCas9 backbone fragment, with a fragment size of 8184 bp, containing an Ori sequence, a chloramphenicol resistance gene (SEQ ID NO: 16), and a dCas9 protein coding sequence (SEQ ID NO: 17), and referred as fragment A (SEQ ID NO: 18), wherein a sequence of pCF-R was set forth in SEQ ID NO: 19; a sequence of pCF-F was set forth in SEQ ID NO: 20;

Second step: the template was removed from the fragment A by using an endonuclease DpnI; after electrophoresis, the same was cleaned by using a PCR purification kit; an appropriate amount of the fragment A was taken, and 2× Seamless Cloning Mix was added for reaction for 30 to 60 minutes;

Third step: a reaction product in the second step was taken to be added into Trans-T1 competent cells, subjected to ice bath and heat shock, and placed on ice for 2 to 5 minutes; an SOC medium was added for incubation; centrifugation was carried out to remove a supernatant. After even mixing, the product was coated to an LB plate containing chloramphenicol. After culture for 12 to 20 hours, single colonies were selected to extract a plasmid to obtain pACYC-dCas9.

### 1.2. Construction of Plasmid Sg-accBC

First step: a Sg-0-RNA plasmid was used as a template to be amplified by using primers pecg-accB-F/pecg-accB-R to obtain an Sg-accBC backbone fragment, with a fragment size of 2239 bp, containing an Ori sequence, sgRNA-scaffold, and an ampicillin resistance gene coding sequence (SEQ ID NO: 21), and referred as fragment B (SEQ ID NO: 22), wherein a sequence of pecg-accB-F was set forth in SEQ ID NO: 23, and a sequence of pecg-accB-R was set forth in SEQ ID NO: 24;

Second step: the template was removed from the fragment B by using an endonuclease DpnI; after electrophoresis, the same was cleaned by using a PCR purification kit; an appropriate amount of the fragment B was taken, and 2× Seamless Cloning Mix was added for reaction for 30 to 60 minutes;

Third step: a reaction product in the second step was taken to be added into Trans-T1 competent cells, subjected to ice bath and heat shock, and placed on ice for 2 to 5 minutes; an SOC medium was added for incubation; centrifugation was carried out to remove a supernatant. After even mixing, the product was coated to an LB plate containing ampicillin. After culture for 12 to 20 hours, single colonies were selected to extract a plasmid to obtain Sg-accBC.

2. Construction of Strain Containing pACYC-dCas9 and Sg-accBC Expression Vectors

1 μL of pACYC-dCas9 and 1 μL of Sg-accBC were taken respectively to be added into 50 μL of MG1655 (DE3) competent cells, and ice bath was carried out for 30 minutes. Heat shock was carried out at 42° C. for 30 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. 20 μL of a bacterial solution was taken to be coated to an LB plate containing chloramphenicol and ampicillin (a final concentration of 50 μg/mL). After overnight culture, 5 positive single colonies were selected for colony PCR validation, to obtain MG1655 (DE3), pACYC-dCas9, Sg-accB positive bacteria (strain A).

### 3. CRISPRi Experiment

Seed culture: 3 to 5 monoclones of the strain A were selected to be inoculated into a 15 mL test tube containing 5 mL of an MOPS medium (containing 20 μg/L glucose), the medium containing chloramphenicol and ampicillin (a final concentration of 50 μg/mL). Culture was carried out at 220 r and 37° C. until the OD₅₅₀ is around 1.5 to 1.8 to be used as a seed solution.

The seed solution was inoculated at a ratio of initial OD₅₅₀=0.1 into an MOPS medium (containing 20 μg/L glucose, 0.1 mM IPTG, and chloramphenicol and ampicillin with a final concentration of 50 μg/mL). After inoculation was completed, shaking for even mixing was carried out. 200 μL of the same was pipetted to a 96-well plate. By using a continuous kinetic assay method, OD₅₅₀ was determined every 10 minutes with a microplate reader (Biotek), to determine the growth curve of the strain.

The results showed that after introduction of interference vectors, pACYC-dCas9 and Sg-accBC, into MG1655 (DE3), the strain was unable to grow in MOPS+2% (w/v) glucose medium due to malonyl-CoA nutritional deficiency (FIG. 7B).

A preparation method for the MOPS medium was as follows:

MOPS medium: 25 mL of a 40×M mother solution, 1 mL of 0.528 M MgCl₂, 1 mL of 0.276 M K₂SO₄, 1 mL of 1 M K₂HPO₄, 100 mL of 200 μg/L glucose, filling to 1 μL with distilled water, and filtration with a 0.22 M sterile filtration membrane for standby use.

A preparation method for the 40×M mother solution was as follows: 334.8 g of MOPS powder, 28.2 g of Tricine, 116.8 g of NaCl, 20.4 g of NH₄Cl, and 32 g of KOH were weighed, distilled water was added to 900 mL, and stirring well was carried out. The pH was adjusted to 7.3 to 7.4 with 10 M HCl or 10 M KOH. While stirring, 2 mL of “trace metal mother solution”, 0.2 mL of ZnCl₂ solution (34 μg/L), 0.2 mL of Na₂SeO₃ solution (43 g/L), and 0.2 mL of Na₂MoO₄ solution (60.5 μg/L) were added into the solution. Distilled water was added to filling to 1 μL. The prepared solution was sub-packed into 20 50 mL centrifuge tubes.

A preparation method for the “trace metal mother solution” was as follows: 5 g of FeCl₂ 4H₂O, 184 mg of CaCl₂·2H₂O, 62 mg of H₃BO₃, 40 mg of MnCl₂·4H₂O, 18 mg of CoCl₂·6H₂O, and 4 mg of CuCl₂·2H₂O were weighed, 70 mL of distilled water was added, 8 mL of concentrated hydrochloric acid (37% HCl) was added, after stirring well, the mixture was transferred to a 100 mL measuring cylinder, and filling to 100 mL with ddH2O was carried out.

(II) Construction and Introduction of NCM Pathway Plasmid (pCDF-bauA-mcrC)
1. Construction of pCDF-bauA-mcrC Plasmid

Step 1, a pCDF-P1-bauA plasmid was used as a template to be amplified by using primers P2-MCRC-R/P2-MCRC-F to obtain a pCDF-P1-bauA vector backbone fragment, with a fragment size of 5129 bp, containing a T7 promoter, an Ori sequence, a bauA coding sequence, and a spectinomycin resistance gene Smr, and referred as fragment III, wherein a sequence of P2-MCRC-R was set forth in SEQ ID NO: 25, a sequence of P2-MCRC-F was set forth in SEQ ID NO: 26, and a sequence of the fragment III was set forth in SEQ ID NO: 27.

An amplification system was as follows: 25 μL of Phanta 2× Buffer, 1 μL of dNTP (10 mM for each dNTP), 20 ng of DNA template, 2 μL of each primer (10 M), 1 μL of Phanta Max Super-Fidelity DNA polymerase, and 18 μL of distilled water, with a total volume of 50 μL.

Amplification conditions were as follows: pre-denaturation at 95° C. for 3 minutes (1 cycle); denaturation at 95° C. for 15 seconds, annealing at 56° C. for 15 seconds, extension at 72° C. for 150 seconds (30 cycles); extension at 72° C. for 5 minutes (1 cycle).

Step 2, a pCDF-P1-McrC plasmid was used as a template to be amplified by using primers MCR-C-pCDF-P2-up/MCR-C-pCDF-P2-down to obtain an McrC coding gene, with a gene fragment size of 2051 bp, and referred as fragment IV, wherein a sequence of the fragment IV was set forth in SEQ ID NO: 28, a sequence of MCR-C-pCDF-P2-up was set forth in SEQ ID NO: 29, and a sequence of MCR-C-pCDF-P2-down was set forth in SEQ ID NO: 30.

An amplification system was as follows: 25 μL of Phanta 2× Buffer, 1 μL of dNTP (10 mM for each dNTP), 20 ng of DNA template, 2 μL of each primer (10 M), 1 μL of Phanta Max Super-Fidelity DNA polymerase, and 18 μL of distilled water, with a total volume of 50 μL.

Amplification conditions were as follows: pre-denaturation at 95° C. for 3 minutes (1 cycle); denaturation at 95° C. for 15 seconds, annealing at 56° C. for 15 seconds, extension at 72° C. for 60 seconds (30 cycles); extension at 72° C. for 5 minutes (1 cycle).

Step 3, the fragment III and the fragment IV were cleaved respectively with an endonuclease DpnI (New England Biolabs (NEB) Inc.) at 37° C. for 30 minutes; after agarose electrophoresis, the same were cleaned by using a PCR purification kit (FastPure Gel DNA Extraction Mini Kit, purchased from Vazyme Biotech Co., Ltd.); an appropriate amount of the fragment III and an appropriate amount of the fragment IV (V_{I}+V_{II}=5 μL, n_{I}/n_{II}=1/3) were taken, and 5 μL of 2× Seamless Cloning Mix (Beyotime Biotechnology Inc.) was added for reaction at 50° C. for 30 minutes.

Step 4, 2 μL of a reaction product in step 3 was taken to be added into 50 μL of Trans-T1 competent cells (purchased from Beijing TransGen Biotech Co., Ltd.), and ice bath was carried out for 30 minutes. Heat shock was carried out at 42° C. for 30 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL). After overnight culture, 5 positive single colonies were selected for colony PCR validation, with primers P2-YZ-up/MCR-C-YZ-down. A clone with a PCR fragment of 662 bp validated as a positive clone was obtained. Two positive clones were selected to extract plasmids for sequencing analysis. By means of comparing sequencing results, a correct plasmid pCDF-bauA-mcrC was obtained. A sequence of MCR-C-YZ-down was set forth in SEQ ID NO: 55; a sequence of P2-YZ-up was set forth in SEQ ID NO: 56.

### 2. Introduction of Malonyl-CoA Deficient Strain in NCM Pathway

1 μL of the pCDF-bauA-mcrC plasmid was taken to be added into 50 μL of MG1655 (DE3) competent cells containing interference vectors pACYC-dCas9 and Sg-accBC, and ice bath was carried out for 30 minutes. Heat shock was carried out at 42° C. for 30 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. 20 μL of a bacterial solution was taken to be coated to an LB plate containing chloramphenicol, ampicillin and spectinomycin (a final concentration of 50 μg/mL). After overnight culture, a target strain (strain B) containing MG1655 (DE3), pACYC-dCas9, Sg-accB, and pCDF-bauA-mcrC was obtained.

Subsequently, growth experiment was carried out:

Seed culture: 3 to 5 (strain B) monoclones were selected to be inoculated into a 15 mL test tube containing 5 mL of an MOPS medium (containing 20 μg/L glucose), the medium containing chloramphenicol, ampicillin and spectinomycin (a final concentration of 50 μg/mL). Culture was carried out at 220 r and 37° C. until the OD₅₅₀ is around 1.5 to 1.8 to be used as a seed solution.

The seed solution was inoculated at a ratio of initial OD₅₅₀=0.1 into an MOPS medium (containing 20 μg/L glucose, 0.1 mM IPTG, and chloramphenicol, ampicillin and spectinomycin with a final concentration of 50 μg/mL). After inoculation was completed, shaking for even mixing was carried out. 200 μL of the same was pipetted to a 96-well plate. By using a continuous kinetic assay method, OD₅₅₀ was determined every 10 minutes with a microplate reader (Biotek), to determine the growth curve of the strain.

The results showed that after the introduction of the NCM pathway (pCDF-bauA-mcrC), the growth damaged by accBC interference was greatly restored (FIG. 7B), indicating that the artificial pathway can replace the natural intracellular pathway for the synthesis of malonyl-CoA.

### Embodiment 6 Use of Malonyl-CoA New Pathway in Fatty Acid Synthesis

Malonyl-CoA is used as an essential extension unit in the biosynthesis of fatty acids. In this embodiment, octanoic acid was selected as the representative of fatty acids, to explore the use of a new synthesis pathway of malonyl-CoA in fatty acid synthesis. The plasmid used for octanoic acid synthesis is pTrc-TE10, which contains thioesterase TE10 from Anaerococcus tetradius and can specifically hydrolyze octanoyl-ACP to form octanoic acid.

Octanoic acid product fermentation was divided into three steps:

First step: 50 ng of the plasmid pCDF-bauA-mcrC (constructed in Embodiment 4) and 50 ng of a thioesterase expression plasmid (pTrc-TE10) were taken to be added into 50 μL of MG1655 (DE3) competent cells (purchased from Shanghai Yuchun Biology Science and Technology Co., Ltd.), and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and ampicillin (a final concentration of 50 μg/mL). After overnight culture, positive colonies, MG1655 (DE3), pCDF-bauA-McrC, pTrc-TE10 (strain C) were obtained.

50 ng of a plasmid pCDF-duet and 50 ng of a plasmid pTrc-TE10 were taken to be added into 50 μL of MG1655 (DE3) competent cells (purchased from Shanghai Yuchun Biology Science and Technology Co., Ltd.), and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and ampicillin (a final concentration of 50 μg/mL). After overnight culture, positive colonies, MG1655 (DE3), pCDF-duet, pTrc-TE10 (as a reference strain C) were obtained.

Second step: seed culture: the strain C and the reference strain C were selected to be respectively inoculated into a 50 mL shake tube of 10 mL of an LB medium containing spectinomycin (a final concentration of 50 μg/mL) and ampicillin (a final concentration of 50 μg/mL). Overnight culture was carried out at 220 r and 37° C.

Third step: fermentation culture:

Fermentation culture: 50 mL of an M9 fermentation medium was prepared in a 250 mL conical flask, containing spectinomycin (a final concentration of 50 μg/mL) and kanamycin (a final concentration of 50 μg/mL). The seed solutions were inoculated with initial OD₅₅₀=0.1. Culture was carried out at 220 r and 30° C. until OD₅₅₀ is around 0.6. IPTG was added until a final concentration was 0.25 mM. Fermentation culture continued at 220 r and 30° C. for 72 hours. OD₅₅₀ determination and sampling were carried out every 24 hours.

The medium used for fermentation was an M9 medium, containing: 20 μg/L glucose, 12.8 μg/L Na₂HPO₄·7H₂O, 0.5 μg/L NaCl, 1 μg/L NH₄Cl, 3 μg/L KH₂PO₄, 2 mM MgSO₄, and 0.1 mM CaCl₂. The preparation method therefor was as follows: 10 mL of 200 μg/L glucose, 20 mL of 5×M9, 200 μL of 1 M MgSO₄, and 10 μL of 1 M CaCl₂ were taken, and then distilled water was added to 100 mL,

wherein 5×M9 contains: 64 μg/L Na₂HPO₄·7H₂O, 15 μg/L KH₂PO₄, 2.5 μg/L NaCl, and 5 μg/L NH₄Cl.

Fatty acid detection: after fermentation was completed, 1 mL of a fermentation broth was taken to be mixed with 125 μL of 10% NaCl (wt/v) and 125 μL of acetic acid; then, 10 μL of a 5 mg/L internal standard (C7:0/C15:0, a final concentration of 50 mg/L) was added, after mixing well momentarily, 500 μL of ethyl acetate was added, and after the samples were subjected to vortex oscillation for 15 seconds, centrifugation was carried out at 16,000×g for 10 minutes. 250 μL of an upper-layer organic phase was transferred to a new glass tube, 2.25 mL of ethanol:hydrogen chloride (30:1 v/v) was added, after incubation was carried out at 55° C. for 1 hour, cooling to a room temperature was carried out, 1.25 mL of ddH2O and 1.25 mL of n-hexane were added, and after vortex oscillation, centrifugation was carried out at 2,000×g for 2 minutes. The upper-layer n-hexane layer was used for GC-MS analysis, and by means of being equipped with Agilent 5975 mass spectrometry and Agilent 7890 gas chromatography, malonyl-CoA was quantified with Gas Chromatography-Flame Ionization Detector/Mass Spectrometer (GC-FID/MS). The analysis program was as follows: an initial temperature of 50° C. was retained for 2 minutes, increased to 200° C. at 25° C./min to be retained for 1 minute, and then increased to 315° C. at 25° C./min to be retained for 2 minutes. Helium was used as a carrier gas, a flow rate was 1 mL/min, and the column used was a DB-5MS separation column (30 m, 0.25 mm ID, 0.25 m, Agilent). Gas chromatography-mass spectrometry detection was carried out.

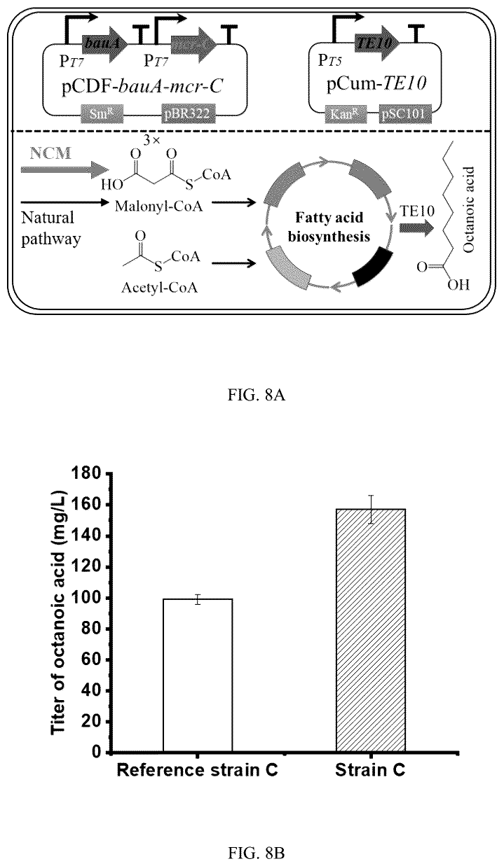

Results show that: compared with the reference strain C (99±3 mg/L octanoic acid), the engineered bacteria (strain C) containing a new malonyl-CoA synthesis pathway show a 58% increase in octanoic acid yield of 157±9 mg/L (FIG. 8B).

### Embodiment 7 Use of Malonyl-CoA New Pathway in Polyketide-Phloroglucinol Synthesis

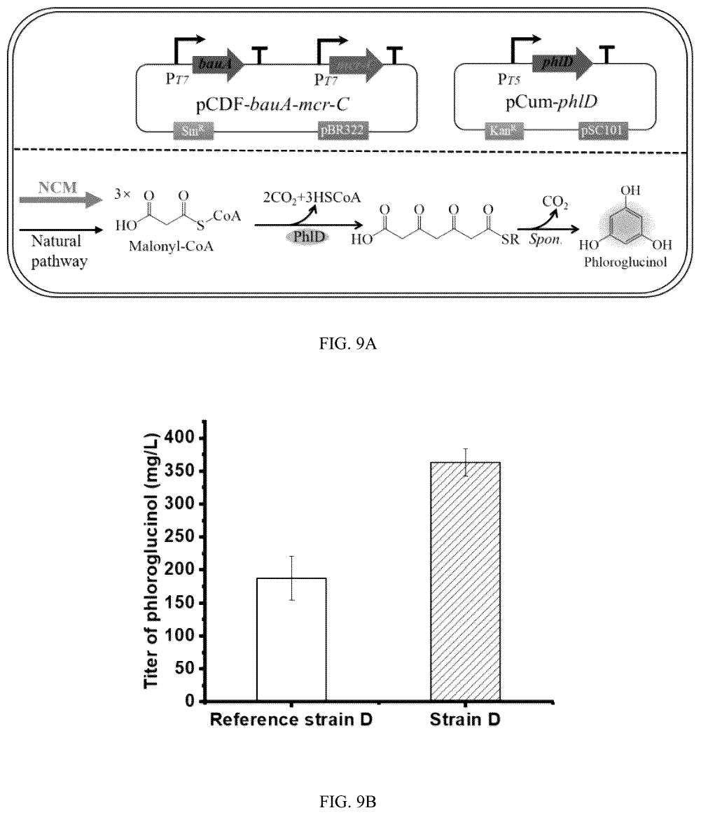

Phloroglucinol is a phenolic compound, can be used as an analgesic, further, can also be used as a precursor for synthesis of bioactive components such as hyperforin, adhyperforin, and rhotomensone, and is widely used in fields such as pharmaceuticals, leather industry, and dye industry (Singh et al. 2009). Type-III PKS (Phloroglucinol synthase, PhlD) can catalyze the biosynthesis of phloroglucinol. Synthesizing 1 molecule of phloroglucinol requires consumption of 3 molecules of malonyl-CoA (FIGS. 9A-9B, with a schematic drawing of a synthesis pathway of phloroglucinol (FIG. 9A), and a histogram of a yield of phloroglucinol (FIG. 9B)).

This experiment required the use of plasmids, pCDF-bauA-mcrC and pCum-phlD.

The construction of the plasmid pCDF-bauA-mcrC was the same as Embodiment 4.

The construction of the plasmid pCum-phlD was divided into four steps:

First step: a pCum plasmid was used as a template to be amplified by using primers pCum-R/pCum-F to obtain a pCum backbone fragment, with a fragment size of 4461 bp, containing an Ori sequence, and a kanamycin resistance gene coding sequence SEQ ID NO: 31), and referred as fragment C (SEQ ID NO: 32), wherein a sequence of pCum-R was set forth in SEQ ID NO: 33; a sequence of pCum-F was set forth in SEQ ID NO: 34;

Second step: a pCDF-phlD plasmid (Genewiz Inc.) was used as a template to be amplified by using primers phlD-up/phlD-down to obtain a PhlD coding gene, with a gene fragment size of 1092 bp, containing a PhlD coding sequence, and referred as fragment D (SEQ ID NO: 35), wherein a sequence of phlD-up was set forth in SEQ ID NO: 36; a sequence of phlD-down was set forth in SEQ ID NO: 37;

Third step: the templates were removed from the fragment C and the fragment D by using an endonuclease DpnI; after electrophoresis, the same was cleaned by using a PCR purification kit; the fragment C and the fragment D were taken, and 2× Seamless Cloning Mix was added for reaction for 30 to 60 minutes;

Fourth step: a reaction product in the third step was taken to be added into Trans-T1 competent cells, subjected to ice bath and heat shock, and placed on ice for 2 to 5 minutes; an SOC medium was added for incubation; centrifugation was carried out to remove a supernatant. After even mixing, the product was coated to an LB plate containing spectinomycin. After culture for 12 to 20 hours, positive single colonies were selected for colony PCR validation, with primers Cum-YZ-up/Cum-YZ-down, to obtain a verification-correct plasmid pCum-phlD, wherein a sequence of Cum-YZ-up was set forth in SEQ ID NO: 57; a sequence of Cum-YZ-down was set forth in SEQ ID NO: 58.

Phloroglucinol product fermentation was divided into three steps:

First step: 50 ng of the plasmid pCDF-bauA-mcrC and 50 ng of the plasmid pCum-phlD were taken to be added into 50 μL of MG1655 (DE3) competent cells, and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and kanamycin (a final concentration of 50 μg/mL). After overnight culture, positive colonies, MG1655 (DE3), pCDF-bauA-mcrC, pCum-phlD (strain D) were obtained.

50 ng of the plasmid pCDF-duet and 50 ng of the plasmid pCum-phlD were taken to be added into 50 μL of MG1655 (DE3) competent cells, and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and kanamycin (a final concentration of 50 μg/mL). After overnight culture, positive colonies, MG1655 (DE3), pCDF-duet, pCum-phlD (as a reference strain D) were obtained.

Second step: seed culture: the strain D and the reference strain D were selected to be respectively inoculated into a 50 mL shake tube of 10 mL of an LB medium containing spectinomycin (a final concentration of 50 μg/mL) and kanamycin (a final concentration of 50 μg/mL). Overnight culture was carried out at 220 r and 37° C. to prepare seed solutions.

Third step: fermentation culture: 50 mL of an M9 fermentation medium was prepared in a 250 mL conical flask, containing spectinomycin (a final concentration of 50 μg/mL) and kanamycin (a final concentration of 50 μg/mL). The seed solutions of the strain D and the reference strain D were inoculated respectively with initial OD₅₅₀=0.1. Culture was carried out at 220 r and 37° C. until OD₅₅₀ is around 0.6. IPTG was added until a final concentration was 0.1 mM, and cumate was added to 0.25 mM. Fermentation culture continued at 220 r and 37° C. for 24 hours.

The preparation of the M9 medium was the same as Embodiment 6.

Analysis method: after fermentation was completed, 2 mL of a sample was taken to be centrifuged at 12,000×g for 2 minutes, and a supernatant was taken for detection with HPLC. A C18 chromatographic column was selected, a flow rate was 0.8 mL/min, a column temperature was 30° C., a mobile phase was 40% acetonitrile, an ultraviolet detector was used for detection, and a wavelength was 254 nm.

Results show that: compared with the yield of phloroglucinol with the reference strain D (187±33 mg/L), the engineered bacteria (strain D) containing a new malonyl-CoA synthesis pathway show a yield of phloroglucinol of 363±21 mg/L, which was 94% higher than the reference strain D (FIG. 9B).

### Embodiment 8 Use of Malonyl-CoA New Pathway in Polyketide-Flaviolin Synthesis

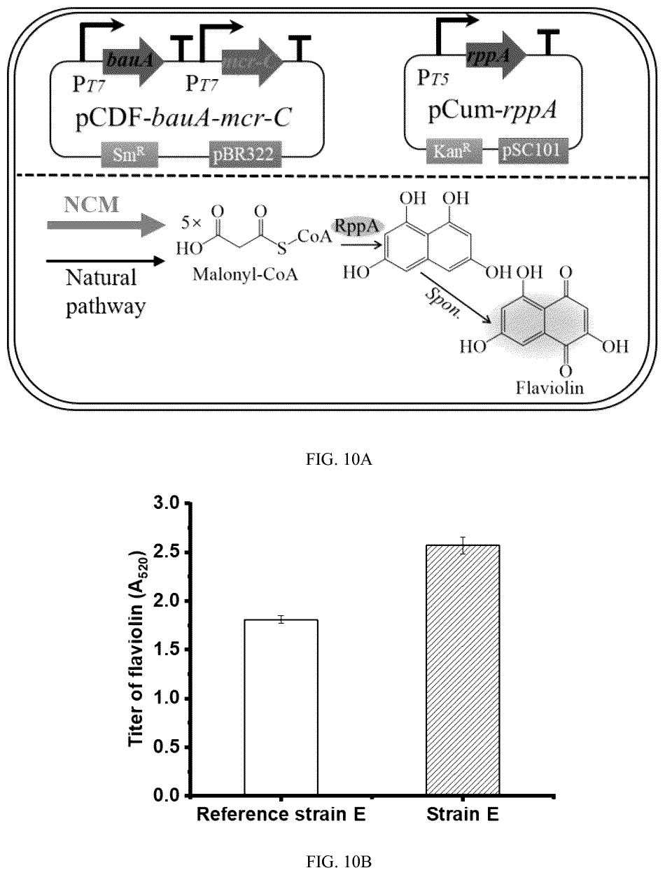

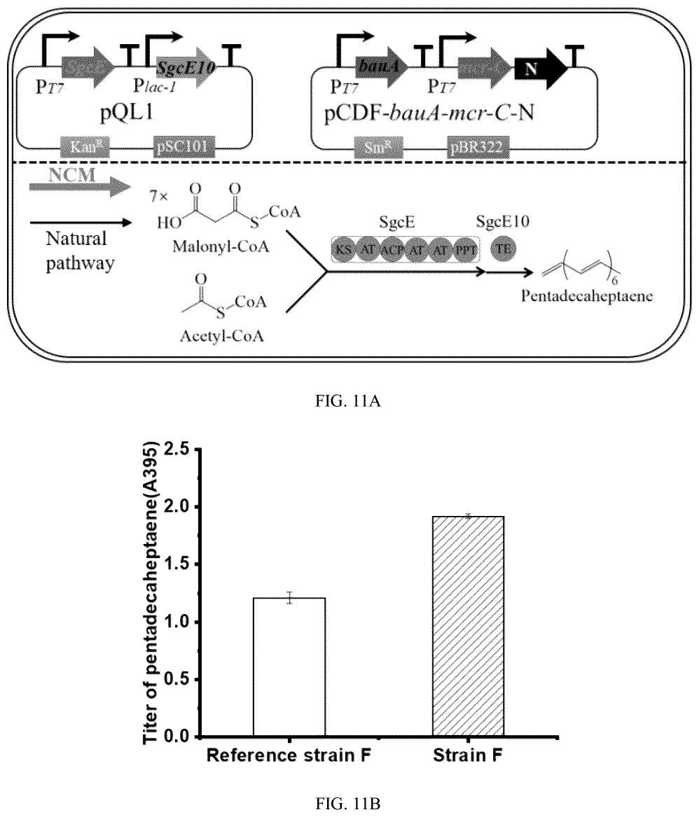

Flaviolin is an important quinone compound, and is an essential precursor for the synthesis of a class of polyketide-isoprenoid hybrid compounds (PIHCs). PIHC compounds can serve as antioxidants, non-steroidal estrogen receptor antagonists, and anti-tumor and anti-cancer drugs. Type-III PKS (RppA) can catalyze the biosynthesis of flaviolin. Synthesizing 1 molecule of flaviolin requires consumption of 5 molecules of malonyl-CoA (FIGS. 10A-10B, with a schematic drawing of a synthesis pathway of flaviolin (FIG. 10A), and a histogram of a yield of flaviolin (FIG. 10B)).

This experiment required the use of plasmids, pCDF-bauA-mcrC and pCum-rppA.

The construction of the plasmid pCDF-bauA-mcrC was shown in Embodiment 4.

The construction of the plasmid pCum-rppA was divided into four steps:

First step: a pCum plasmid was used as a template to be amplified by using primers pCum-R/pCum-F to obtain a pCum backbone fragment, with a fragment size of 4461 bp, containing an Ori sequence, and a kanamycin resistance gene coding sequence (SEQ ID NO: 31), and referred as fragment C (SEQ ID NO: 32), wherein a sequence of pCum-R was set forth in SEQ ID NO: 33; a sequence of pCum-F was set forth in SEQ ID NO: 34;

Second step: a pCDF-rppA plasmid (Genewiz Inc.) was used as a template to be amplified by using primers rppA-Cum-up/rppA-Cum-down to obtain an RppA coding gene, with a gene fragment size of 1169 bp, containing a RppA coding sequence, and referred as fragment E (SEQ ID NO: 38), wherein a sequence of rppA-Cum-up was set forth in SEQ ID NO: 39; a sequence of rppA-Cum-down was set forth in SEQ ID NO: 40;

Third step: the templates were removed from the fragment C and the fragment E by using an endonuclease DpnI; after electrophoresis, the same was cleaned by using a PCR purification kit; the fragment C and the fragment E were taken, and 2× Seamless Cloning Mix was added for reaction for 30 to 60 minutes;

Fourth step: a reaction product in the third step was taken to be added into Trans-T1 competent cells, subjected to ice bath and heat shock, and placed on ice for 2 to 5 minutes; an SOC medium was added for incubation; centrifugation was carried out to remove a supernatant. After even mixing, the product was coated to an LB plate containing kanamycin. After culture for 12 to 20 hours, positive single colonies were selected for colony PCR validation, with primers Cum-YZ-up/Cum-YZ-down, to obtain a verification-correct plasmid pCum-rppA.

Flaviolin product fermentation was divided into three steps:

First step: 50 ng of the plasmid pCDF-bauA-mcrC and 50 ng of the plasmid pCum-rppA were taken to be added into 50 μL of MG1655 (DE3) competent cells, and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and kanamycin (a final concentration of 50 μg/mL). After overnight culture, positive colonies, MG1655 (DE3), pCDF-bauA-mcrC, pCum-rppA (strain E) were obtained.

50 ng of the plasmid pCDF-duet and 50 ng of the plasmid pCum-rppA were taken to be added into 50 μL of MG1655 (DE3) competent cells, and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and kanamycin (a final concentration of 50 μg/mL). After overnight culture, positive colonies, MG1655 (DE3), pCDF-duet, pCum-rppA (as a reference strain E) were obtained.

Second step: seed culture: the strain E and the reference strain E were selected to be respectively inoculated into a 50 mL shake tube of 10 mL of an LB medium containing spectinomycin (a final concentration of 50 μg/mL) and kanamycin (a final concentration of 50 μg/mL). Overnight culture was carried out at 220 r and 37° C.

Third step: fermentation culture: 50 mL of an M9 fermentation medium was prepared in a 250 mL conical flask, containing spectinomycin (a final concentration of 50 μg/mL) and kanamycin (a final concentration of 50 μg/mL). The seed solutions of the strain E and the reference strain E were inoculated respectively with initial OD₅₅₀=0.1. Culture was carried out at 220 r and 37° C. until OD₅₅₀ is around 0.6. IPTG was added until a final concentration was 0.1 mM, and cumate was added to 0.25 mM. Fermentation culture continued at 220 r and 37° C. for 24 hours.

The preparation of the M9 medium was the same as Embodiment 6.

Analysis method: after fermentation was completed, 2 mL of a sample was taken to be centrifuged at 12,000×g for 2 minutes, a supernatant was taken, and the absorbance value (A520) of the supernatant at 520 nm was determined with a spectrophotometer.

Results show that: compared with the reference strain E (A520=1.81±0.04), the engineered bacteria (strain E) containing a new malonyl-CoA synthesis pathway show a 43% increase in flaviolin yield (A520=2.57±0.09) (FIG. 10B).

### Embodiment 9 Use of Malonyl-CoA New Pathway in Polyketide-Pentadecaheptaene Synthesis

Pentadecaheptaene is a class of olefinic compounds, and is not only a key intermediate for the biosynthesis of enediyne (a famous anticancer antibiotic), but also an ideal biofuel with high energy density. Pentadecaheptaene is synthesized by type-I PKS SgcE and homologous thioesterase SgcE10. Synthesizing 1 molecule of pentadecaheptaene requires 1 molecule of acetyl-CoA and 7 molecules of malonyl-CoA as precursors (FIGS. 11A-11B, with a schematic drawing of a synthesis pathway of pentadecaheptaene (FIG. 11A), and a histogram of a yield of pentadecaheptaene production (FIG. 11B)

). PQL1 is a previously constructed plasmid containing the pentadecaheptaene biosynthesis pathway (Q Liu et al., 2015).

Pentadecaheptaene product fermentation was divided into three steps:

First step: 50 ng of the plasmid pCDF-bauA-mcrC and 50 ng of pQL1 were taken to be added into 50 μL of BL21 (DE3) competent cells (purchased from Shanghai Yuchun Biology Science and Technology Co., Ltd.), and ice bath was carried out for 30 minutes. Heat shock was carried out at 42° C. for 60 seconds, and the same was immediately placed on ice for 2 minutes. 500 μL of an SOC medium was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and kanamycin (a final concentration of 50 μg/mL). After overnight culture, positive colonies, BL21 (DE3), pCDF-bauA-mcrC, pQL1 (strain F) were obtained.

50 ng of a plasmid pCDF-duet and 50 ng of pQL1 were taken to be added into 50 μL of BL21 (DE3) competent cells (purchased from Shanghai Yuchun Biology Science and Technology Co., Ltd.), and ice bath was carried out for 30 minutes. Heat shock was carried out at 42° C. for 60 seconds, and the same was immediately placed on ice for 2 minutes. 500 μL of an SOC medium was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL) and kanamycin (a final concentration of 50 μg/mL). After overnight culture, positive colonies, BL21 (DE3), pCDF-bauA-mcrC, pQL1 (as a reference strain F) were obtained.

Second step: seed culture: monoclones of the strain F and the reference strain F were selected to be respectively inoculated into a 50 mL shake tube of 10 mL of an LB medium containing spectinomycin (a final concentration of 50 μg/mL) and kanamycin (a final concentration of 50 μg/mL). Overnight culture was carried out at 220 r and 37° C.

Third step: fermentation culture: 50 mL of an LB fermentation medium was prepared in a 250 mL conical flask, containing spectinomycin (a final concentration of 50 μg/mL) and kanamycin (a final concentration of 50 μg/mL). The seed solutions of the strain F and the reference strain F were inoculated respectively with initial OD₅₅₀=0.1. Culture was carried out at 220 r and 37° C. until OD₅₅₀ is around 0.6. IPTG was added until a final concentration was 0.1 mM. Timing is 0 o'clock when adding an inducer, and fermentation culture continued at 220 r and 18° C. for 24 hours.

Analysis method: 5 mL of a sample was taken to be extracted with ethyl acetate, and the absorbance value (A395) of the supernatant at 395 nm was determined with a spectrophotometer.

Results show that: compared with the reference strain F (A395=1.21±0.05), the engineered bacteria (strain F) containing a new malonyl-CoA synthesis pathway show a 59% increase in pentadecaheptaene yield (A395=1.92±0.02) (FIG. 11B).

### Embodiment 10 Use of Malonyl-CoA New Pathway in Polyketide-Natamycin Synthesis

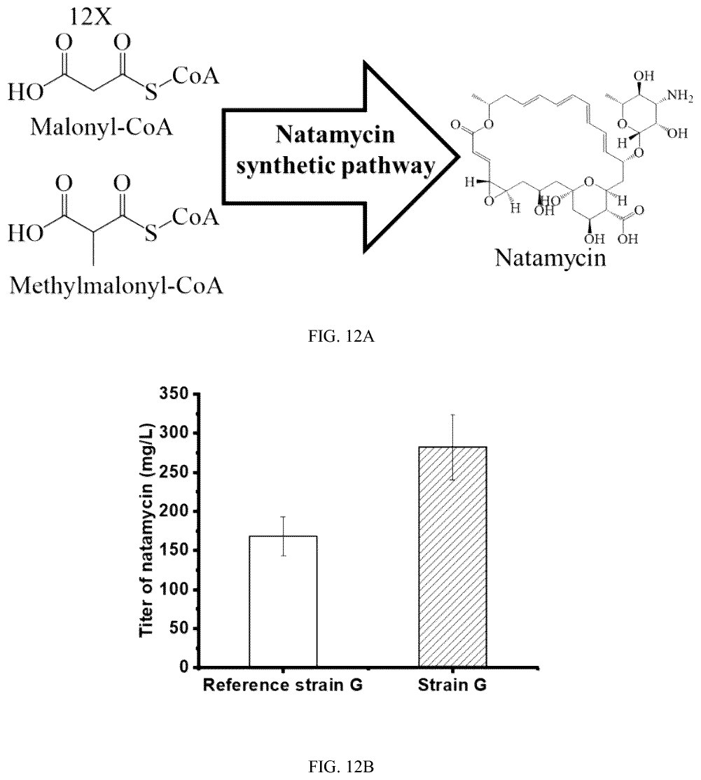

Natamycin is a polyketide, is a class of representative aminoglycoside antibiotics, and is the only antifungal drug approved by the U.S. Food and Drug Administration (FDA). Streptomyces gilvosporeus is the main strain producing natamycin. In this strain, the biosynthesis of natamycin is initiated by type-I PKS. Synthesizing 1 molecule of natamycin requires 1 molecule of methylmalonyl-CoA and 12 molecules of malonyl-CoA as precursors (FIGS. 12A-12B, with a schematic drawing of a synthesis pathway of natamycin (FIG. 12A), and a histogram of a yield of natamycin production (FIG. 12B)).

1. Construction of Plasmid pSET152-KasOP-BauA-SPL42-McrC:

1.1. A plasmid pSET152 was cleaved with XbaI and BamHI at 37° C. for 2 hours, and after agarose gel electrophoresis, a pSET152 backbone fragment 5709 bp was recovered, and referred as fragment F (SEQ ID NO: 41).

1.2. A pLH10 plasmid was used as a template to be amplified by using primers KasOP-F/R to obtain a KasOP promoter fragment, referred as fragment G (SEQ ID NO: 42); a pLH2 plasmid was used as a template to be amplified by using primers SPL42-F/R to obtain a promoter SPL42 promoter fragment, referred as fragment H (SEQ ID NO: 43); a pCDF-bauA-McrC plasmid was used as a template to be amplified by using primers BauA-F/R to obtain a BauA gene fragment, referred as fragment J (SEQ ID NO: 44), and to be amplified by using primers McrC-F/R to obtain an McrC gene fragment, referred as fragment K (SEQ ID NO: 45), wherein a sequence of KasOP-F was set forth in SEQ ID NO: 46; a sequence of KasOP-R was set forth in SEQ ID NO: 47; a sequence of SPL42-F was set forth in SEQ ID NO: 48; a sequence of SPL42-R was set forth in SEQ ID NO: 49; a sequence of BauA-F was set forth in SEQ ID NO: 50; a sequence of BauA-R was set forth in SEQ ID NO: 51; a sequence of McrC-F was set forth in SEQ ID NO: 52; a sequence of McrC-R was set forth in SEQ ID NO: 53.

An amplification system was as follows: 25 μL of Phanta 2× Buffer, 1 μL of dNTP (10 mM for each dNTP), 20 ng of DNA template, 2 μL of each primer (10 M), 1 μL of Phanta Max Super-Fidelity DNA polymerase, and 18 μL of distilled water, with a total volume of 50 μL.

Amplification conditions were as follows: pre-denaturation at 95° C. for 3 minutes (1 cycle); denaturation at 95° C. for 15 seconds, annealing at 56° C. for 15 seconds, extension at 72° C. for 60 seconds (30 cycles); extension at 72° C. for 5 minutes (1 cycle).

1.3. By using KasOP-F/BauA-R as primers, and using the fragment H and fragment M as templates, the fragment H and the fragment M were linked with OE-PCR to be referred as fragment P; and by using SPL42-F/McrC-R as primers, and using the fragment K and fragment N as templates, the fragment K and the fragment N were linked with OE-PCR to be referred as fragment Q.

1.4. An appropriate amount of fragment G, fragment P, and fragment Q were taken, and 5 μL of 2× Seamless Cloning Mix (Beyotime Biotechnology Inc.) was added for reaction at 50° C. for 30 minutes.

1.5. 2 μL of a reaction product in the fourth step was taken to be added into 50 μL of Trans-T1 competent cells (purchased from Beijing TransGen Biotech Co., Ltd.), and ice bath was carried out for 30 minutes. Heat shock was carried out at 42° C. for 30 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing apramycin (a final concentration of 50 μg/mL). After overnight culture, 5 positive single colonies were selected for colony PCR validation, with primers BauA-F/R and McrC-F/R. A clone with PCR fragments of 1368 bp and 2057 bp validated as a positive clone was obtained. Two positive clones were selected to extract plasmids for sequencing analysis. By means of comparing sequencing results, a correct plasmid pSET152-KasOP-BauA-SPL42-McrC was obtained.

### Construction of Strain Overexpressing the NCM Pathway:

2. Processing of E. coli:

2.1. 50 ng of the plasmid pSET152-KasOP-BauA-SPL42-McrC was taken to be added into E. coli ETU12567/pUZ8002 competent cells, and ice bath was carried out for 30 minutes. Heat shock was carried out at 42° C. for 30 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing apramycin (a final concentration of 50 μg/mL), kanamycin (a final concentration of 50 μg/mL), and chloramphenicol (a final concentration of 34 μg/mL). After overnight culture, strain H was obtained.

2.2. 3 to 5 of single colonies of the strain H were selected to a 5 mL LB medium containing apramycin (a final concentration of 50 μg/mL), kanamycin (a final concentration of 50 μg/mL), and chloramphenicol (a final concentration of 34 μg/mL). Overnight culture was carried out at 220 rpm and 37° C.

2.3. The above bacterial solution of the strain H was taken to be inoculated with an inoculum size of 10% to a 5 mL LB medium containing apramycin (a final concentration of 50 μg/mL), kanamycin (a final concentration of 50 μg/mL), and chloramphenicol (a final concentration of 34 μg/mL). Culture was carried out for around 2.5 hours, and shaking was carried out until OD=0.6 to 0.8.

2.4. Centrifugation was carried out at 3500 rpm for 5 minutes to collect a bacterial cell, which was washed 2 to 3 times by adding 5 mL of an LB medium, the supernatant was removed, 1 to 2 mL of an LB medium was added to resuspend the bacterial cell for standby use, to obtain a standby bacterial solution of the strain H.

3. Processing of Streptomyces gilvosporeus J1-002 Strain:

3.1. 100 μL of a Streptomyces gilvosporeus J1-002 bacterial solution was taken to be coated on an SFM plate, and put in a 30° C. incubator for culture for around 7 days.

3.2. All the spores were scraped using a cotton swab into a sterile 50 mL centrifuge tube, 6 mL of TES was added for resuspension, and heat shock was carried out at 50° C. for 10 minutes.

3.3. 6 mL of a 2× spore pre-germination solution and CaCl₂ with a final concentration of 10 mM were added, pre-germination was carried out at 220 rpm and 37° C. for 2 to 3 hours, the product was sub-packed into EP tubes at 2 mL/tube, and centrifugation was carried out to remove a supernatant for standby use.

3.4. The prepared bacterial solution of the strain H and the Streptomyces gilvosporeus J1-002 spore solution were mixed in different proportions, coated to an SFM plate containing 10 mM Mg²⁺, and put in a 30° C. incubator for culture for 14 to 16 hours.

3.5. By means of calculation with a solid plate volume of 25 mL, an antibiotic with trimethoprim with a final concentration of 50 μg/mL and Apr with a final concentration of 50 μg/mL was added into 2 mL of sterile water, which covers the plate after mixing well. The plate was put in a 30° C. incubator for 7 to 9 days before zygotes grew. SFM medium: 20 μg/L soybean cake powder, 20 μg/L mannitol, and 16 μg/L Qingdao agar, pH of 7.2, and sterilization at 115° C. for 30 minutes. 2× spore pre-germination solution: 0.1 μg/L Difco Yeast Extract, 0.1 μg/L Difco Casmino acid, and sterilization at 115° C. for 30 minutes.

### Screening of Engineered Strains:

First step: the zygotes in step 3.5 were selected to be transferred to an SFM plate containing trimethoprim with a final concentration of 50 μg/mL and apramycin with a final concentration of 50 μg/mL for propagation, and culture carried out in a 30° C. incubator for 5 to 7 days.

Second step: some spores were taken by scraping to extract a genome for PCR validation. Genome extraction: some spores were taken by scraping to 500 μL of SET Buffer, and 20 μL of a 100 mg/mL lysozyme was then added to work in a 37° C. water bath for 60 minutes to lyse the bacterial cell; 100 μL of a 10% SDS solution was added for upside-down mixing well; 600 μL of a DNA extraction phenol reagent was added, and after upside-down mixing well, centrifugation was carried out at 12000 rpm for 5 minutes; the supernatant was transferred to a new 1.5 mL centrifuge tube, 600 μL of a DNA extraction phenol reagent was added, and after upside-down mixing well, centrifugation was carried out at 12000 rpm for 5 minutes; 50 μL of 3M NaAc was added, after upside-down mixing well, 500 μL of pre-cooled isopropanol was added, and after thorough mixing well, centrifugation was carried out at 12000 rpm for 5 minutes; the supernatant was removed, and the product was washed twice with 75% ethanol; after the ethanol evaporates, appropriate distilled water was added to dissolve the genome. By using Apr-F/R as primers and the genome as a template, when the amplified band was 411 bp, it was a correct engineered strain J1-002, pSET152-KasOP-BauA-SPL42-McrC (strain G). J1-002 was a reference strain G. A sequence of Apr-F was set forth in SEQ ID NO: 59; a sequence of Apr-R was set forth in SEQ ID NO: 60.

The formula of SET Buffer: 75 mM NaCl, 25 mM EDTA, 20 mM Tris, pH of 8.0.

Natamycin Fermentation was Divided into Three Steps:

First step: plate culture: 100 μL of the strain G and 100 μL of a bacterial solution of the reference strain G were coated to a resistant SFM plate containing apramycin (a final concentration of 50 μg/mL), and put in a 30° C. incubator for culture for around 10 days until the spores were gray white.

Second step: seed culture: approximately 1 cm² of an agar block was taken from the plate to be inoculated into a 100 mL (500 mL shake flask) seed medium, and culture was carried out at 200 rpm and 28° C. for 24 hours.

Third step: shake flask fermentation: the seed solution was inoculated with an inoculum size of 5% into a fermentation shake flask, with a liquid volume of 80 mL (500 mL shake flask), and culture was carried out at 200 rpm and incubated at 28° C. for 5 days.

Seed medium: 12 μg/L yeast powder, 10 μg/L NaCl, 15 μg/L glucose, 0.2 μg/L defoamer, pH of 6.8 to 7.0, and sterilization at 115° C. for 30 minutes. Fermentation medium: 8 μg/L yeast powder, 30 μg/L soy protein isolate, 40 μg/L glucose, 0.001 μg/L biotin, pH of 7.1, and sterilization at 115° C. for 30 minutes.

An analysis method was as follows: 1 mL of a fermentation broth was taken, 9 mL of methanol was added, after thorough shaking, sonication was carried out for 20 minutes, and then centrifugation was carried out at 8000 rpm for 15 minutes. The supernatant was diluted to an appropriate multiple with methanol and then filtered with a 0.22 m microporous filtration membrane. HPLC detection method: mobile phase: methanol:water=6:4; flow rate: 0.7 mL/min; detection wavelength: 303 nm; column temperature: 30° C.

Results show that: compared with the reference strain G (168±25 mg/L), the engineered bacteria (strain G) containing a new malonyl-CoA synthesis pathway show a 67% increase in natamycin yield (282±42 mg/L) (FIG. 12B).

### Embodiment 11 Use of Malonyl-CoA New Pathway in Polyketide-Spinosad Synthesis

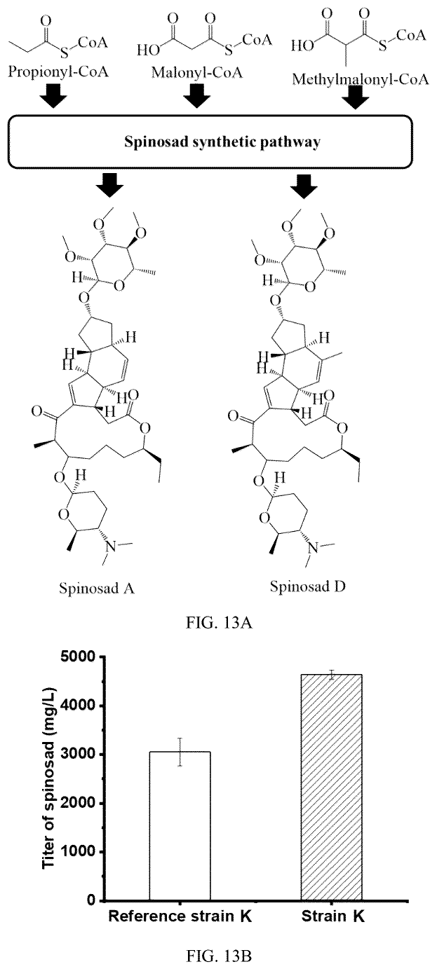

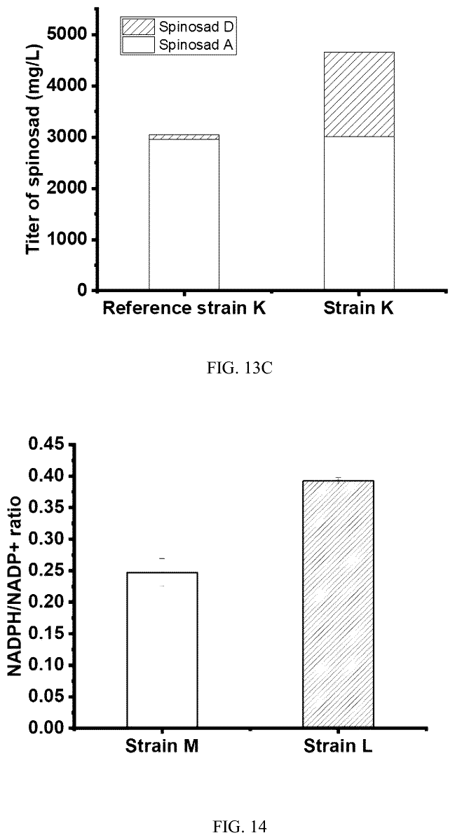

Spinosad is an efficient, safe, and broad-spectrum biological insecticide. Saccharopolyspora spinosa is the main strain producing spinosad. In this strain, the biosynthesis of spinosad is initiated by type-I PKS. Synthesizing 1 molecule of spinosad requires 1 molecule of propionyl-CoA, 1 molecule of methylmalonyl-CoA, 9 molecules of malonyl-CoA, and 1 molecule of propionyl-CoA as precursors (FIGS. 13A-13C, with a schematic drawing of a synthesis pathway of spinosad (FIG. 13A), and a histogram of a yield of spinosad production (FIGS. 13B-13C)).

Construction of Saccharopolyspora spinosa Overexpressing the NCM Pathway:
1. Processing of E. coli:

1.1. 50 ng of the plasmid pSET152-KasOP-BauA-SPL42-McrC (the construction method was the same as Embodiment 10) was taken to be added into E. coli DH10B competent cells, and ice bath was carried out for 30 minutes. Heat shock was carried out at 42° C. for 30 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing apramycin (a final concentration of 50 μg/mL), to obtain strain J; E. coli ET pUB307 was streaked to an LB plate containing kanamycin (a final concentration of 50 μg/mL) and chloramphenicol (a final concentration of 34 μg/mL) for overnight culture.

1.2. 3 to 5 of single colonies of the strain J were selected to a 5 mL LB medium containing apramycin (a final concentration of 50 μg/mL). 3 to 5 of ET pUB307 single colonies were selected to a 5 mL LB medium containing kanamycin (a final concentration of 50 μg/mL) and chloramphenicol (a final concentration of 34 μg/mL). Overnight culture was carried out at 220 rpm and 37° C.

1.3. The above bacterial solution was taken to be inoculated with an inoculum size of 10% to a 50 mL LB medium containing apramycin (a final concentration of 50 μg/mL), and a 50 mL LB medium containing kanamycin (a final concentration of 50 μg/mL) and chloramphenicol (a final concentration of 34 μg/mL). Culture was carried out for around 2.5 hours, and shaking was carried out until OD=0.6 to 0.8.

1.4. Centrifugation was carried out at 3500 rpm for 5 minutes to collect a bacterial cell, which was washed 2 to 3 times by adding 20 mL of an LB medium, the supernatant was removed, and 3 to 4 mL of an LB medium was added for resuspension for standby use.

2. Processing of Saccharopolyspora spinosa WHU1107:

2.1. 100 μL of a Saccharopolyspora spinosa WHU1107 bacterial solution prepared in step 1.4 was pipetted to be coated on an ABB13 plate, and put in a 28° C. incubator for culture for 7 days.

2.2. 1 cm² of a bacterial block was selected using a pipette tip to a TSB-M medium for primary seed culture at 220 rpm and 28° C. for 72 hours.

2.3. The primary seed solution was transferred to secondary seeds at a transfer amount of 2%, and culture was carried out at 220 rpm and 28° C. for 48 hours.

2.4. 50 mL of the secondary seed solution was poured into a 50 mL centrifuge tube, and centrifugation was carried out at 4500 rpm for 10 minutes for collecting a bacterial cell. The bacterial cell was washed 2 to 3 times with 20 mL of an LB medium, and finally, approximately 5 to 8 mL of an LB medium was added to resuspend the bacterial cell for standby use.

2.5. The Saccharopolyspora spinosa WHU1107 bacterial solution was evenly mixed with and strain H and ET pUB307 in different proportions to be evenly coated to an ABB13 plate containing 10 mM Mg²⁺. The plate was put in a 28° C. incubator for culture for 22 hours.

2.6. By means of calculation with a solid plate volume of 25 mL, an antibiotic with trimethoprim with a final concentration of 50 μg/mL and Apr with a final concentration of g/mL was added into 2 mL of sterile water, which covers the plate after mixing well. The plate was put in a 28° C. incubator for culture for 10 days before zygotes grew.

ABB13 medium: 5 μg/L soluble starch, 5 μg/L soya peptone, 2.1 μg/L 3-CN-morpholine propanesulfonic acid, 3 μg/L calcium carbonate, 0.01 μg/L thiamine hydrochloride, 0.046 μg/L ferrous sulfate heptahydrate, 20 μg/L agar, pH of 7.0, and sterilization at 115° C. for 30 minutes. TSB-M medium: 30 μg/L tryptone soya broth, and 50 g/L mannitol; sub-packing 50 mL of a medium in a 250 shake flask, and sterilization at 115° C. for 30 minutes.

### 3. Screening of Engineered Strains:

First step: the zygotes in step 2.6 were selected to be transferred to an ABB13 plate containing trimethoprim with a final concentration of 50 μg/mL and apramycin with a final concentration of 15 μg/mL for propagation, and culture carried out in a 28° C. incubator for 5 to 7 days.

Second step: some spores were taken by scraping to extract a genome for PCR validation. Genome extraction: some spores were taken by scraping to 500 μL of SET Buffer, and 20 μL of a 100 mg/mL lysozyme was then added to work in a 37° C. water bath for 60 minutes to lyse the bacterial cell; 100 μL of a 10% SDS solution was added for upside-down mixing well; 600 μL of a DNA extraction phenol reagent was added, and after upside-down mixing well, centrifugation was carried out at 12000 rpm for 5 minutes; the supernatant was transferred to a new 1.5 mL centrifuge tube, 600 μL of a DNA extraction phenol reagent was added, and after upside-down mixing well, centrifugation was carried out at 12000 rpm for 5 minutes; 50 μL of 3M NaAc was added, after upside-down mixing well, 500 μL of pre-cooled isopropanol was added, and after thorough mixing well, centrifugation was carried out at 12000 rpm for 5 minutes; the supernatant was removed, and the product was washed twice with 75% ethanol; after the ethanol evaporates, appropriate distilled water was added to dissolve the genome. By using Apr-F/R as primers and the genome as a template, when the amplified band was 411 bp, it was a correct engineered strain WHU1107, pSET152-KasOP-BauA-SPL42-McrC (strain K). (WHU1107 was a reference strain K)

The formula of SET Buffer: 75 mM NaCl, 25 mM EDTA, 20 mM Tris, pH of 8.0.

Spinosad fermentation was divided into fourth steps:

First step: plate culture: 100 μL of the strain K and 100 μL of a bacterial solution of the reference strain K were coated to a resistant fermentation plate containing apramycin (a final concentration of 15 μg/mL), and put in a 28° C. incubator for culture for 7 days.

Second step: primary seed culture: approximately 1 cm² of a bacterial block was streaked to a seed medium, and culture was carried out at 250 rpm and 28° C. with a humidity of 60% for 96 hours.

Third step: secondary seed culture: the primary seeds were transferred to secondary seeds at a transfer amount of 1%, and culture was carried out at 250 rpm and 28° C. with a humidity of 60% for 60 hours.

Fourth step: fermentation: the secondary seeds were transferred to a fermentation medium at a transfer amount of 5%, and culture was carried out at 250 rpm and 28° C. with a humidity of 60% for 12 days.

Fermentation plate: 5 μg/L glucose, 3 μg/L yeast extract, 10 μg/L enzyme-hydrolized casein (N-Z amine type A), 20 μg/L agar, pH of 7.0, and sterilization at 115° C. for 30 minutes. Seed medium: 10 μg/L glucose, 10 μg/L yeast extract, 2 μg/L enzyme-hydrolized casein (N-Z amine type A), 25 μg/L cottonseed cake powder, 20 μg/L corn starch, 2 μg/L magnesium sulfate heptahydrate, 1 μg/L ammonium sulfate, pH of 7.0, sub-packing 25 mL of a medium in a 250 mL shake flask, and sterilization at 121° C. for 30 minutes. Fermentation medium: 80 μg/L glucose, 20 μg/L cottonseed cake powder, 10 μg/L Dolphin brand protein powder, 5 μg/L yeast powder, 4 μg/L trisodium citrate, 2 μg/L dipotassium hydrogen phosphate, 3 μg/L calcium carbonate, 2 μg/L ammonium sulfate, 50 μg/L rapeseed oil, pH of 7.0, sub-packing 25 mL of a medium in a 250 mL shake flask, and sterilization at 121° C. for 30 minutes.

A detection method was as follows: 1 mL of a fermentation broth was taken, 4 mL of ethanol was added, after thorough shaking, centrifugation was carried out at 8000 rpm for 15 minutes. The supernatant was diluted to an appropriate multiple with ethanol and then filtered with a 0.22 m microporous filtration membrane. HPLC detection method: mobile phase: methanol:acetonitrile:water (containing 5% ammonium acetate)=45:45:10; flow rate: 1 mL/min; detection wavelength: 250 nm; column temperature: 30° C.

Results show that: introducing the new NCM pathway into the Saccharopolyspora spinosa WHU1107 strain (CCTCC NO: M 2021307) results in a 52% increase in yield, and a change in the composition of the products of spinosad A and spinosad D. Original spinosad A:spinosad D is changed from 97:3 to 65:35 (FIGS. 13B-13C).

### Embodiment 12 the New Pathway Improves Production of NADPH Cofactors

1. Obtaining a strain: 50 ng of a plasmid pCDF-bauA-mcrC and 50 ng of a plasmid pCDF-duet were taken to be respectively added into 50 μL of MG1655 (DE3) competent cells (purchased from Shanghai Yuchun Biology Science and Technology Co., Ltd.), and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL). After overnight culture, positive colonies were obtained, to obtain engineered bacteria MG1655 (DE3), pCDF-bauA-mcrC (strain L) and a reference strain MG1655(DE3), pCDF-duet (strain M).

### 2. Preparation of Bacterial Solution

Seed culture: After sterilization of each component of a fermentation medium, a seed medium was prepared in a super clean bench. Monoclones of the strain M and monoclones of the strain L were selected respectively to be inoculated into a 50 mL shake tube of 5 mL of a seed medium containing spectinomycin (a final concentration of 50 μg/mL). Overnight culture was carried out at 220 r and 37° C.

Fermentation culture: 50 mL of a fermentation medium was prepared in a 250 mL conical flask, containing spectinomycin (a final concentration of 50 μg/mL). The seed solution was inoculated with initial OD₅₅₀=0.1, IPTG was added until a final concentration was 0.1 mM. Culture was carried out in a constant-temperature shaker at 220 r and 37° C. until OD₅₅₀ was around 1.1 mL of a bacterial solution of the strain M and 1 mL of a bacterial solution of the strain L were pipetted.

### 3. Determination of NADPH

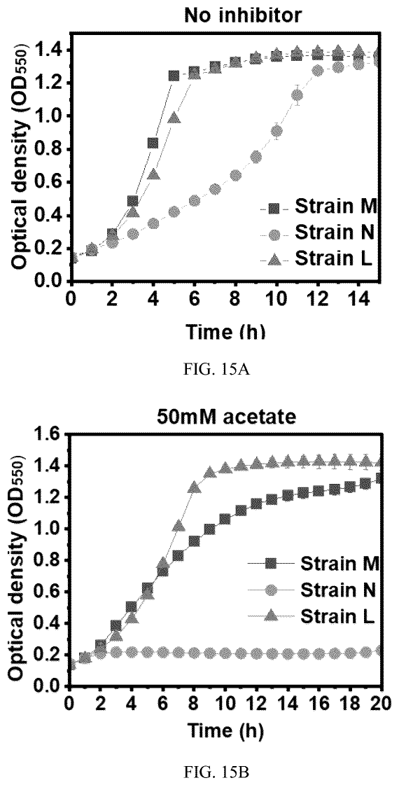

The NADPH/NADP⁺ of the bacterial solutions was determined using an NADPH/NADP⁺ detection kit (WST-8 method) (purchased from Beyotime Biotechnology Inc.). Results show that: compared with the reference strain M (0.247±0.022), the engineered bacteria L containing a new malonyl-CoA synthesis pathway show a 59% increase in intracellular NADPH/NADP⁺ (0.393±0.005) (FIG. 14).

### Embodiment 13 the New Pathway does not Produce Cytotoxicity

Numerous studies have shown that overexpression of ACC in natural synthesis pathways often leads to cytotoxicity, thereby inhibiting cell growth. Next, we tested the effects of ACC and NCM on the growth of representative E. coli MG1655 (DE3).

Medium used for tolerance testing: IPTG (a final concentration of 0.1 mM) and spectinomycin (a final concentration of 50 μg/mL) were added into an MOPS medium (containing 20 μg/L glucose).

This experiment required the use of a plasmid, pCDF-ACC.

The construction of the plasmid pCDF-ACC was divided into eight steps:

First step: A pCDF-duet plasmid was used as a template to be amplified by using primers P1-R/P1-F to obtain a pCDF vector backbone fragment, with a fragment size of 3781 bp, containing a T7 promoter, an Ori sequence, and a spectinomycin resistance gene Smr, and referred as fragment I, wherein a sequence of P1-R was set forth in SEQ ID NO: 1, a sequence of P1-F was set forth in SEQ ID NO: 2, and a sequence of the fragment I was set forth in SEQ ID NO: 3.

Second step: a Corynebacterium glutamicum genome was used as a template to be amplified by using primers accBC-up/accBC-down to obtain an accBC coding gene, with a gene fragment size of 1811 bp, containing an accBC coding sequence, and referred as fragment R (SEQ ID NO: 61), wherein a sequence of accBC-up was set forth in SEQ ID NO: 62; a sequence of accBC-down was set forth in SEQ ID NO: 63.

Third step: the templates were removed from the fragment I and the fragment R by using an endonuclease DpnI; after electrophoresis, the same was cleaned by using a PCR purification kit; the fragment I and the fragment R were taken, and 2× Seamless Cloning Mix was added for reaction for 30 to 60 minutes.

Fourth step: a reaction product in the third step was taken to be added into Trans-T1 competent cells, subjected to ice bath and heat shock, and placed on ice for 2 to 5 minutes; an SOC medium was added for incubation; centrifugation was carried out to remove a supernatant. After even mixing, the product was coated to an LB plate containing spectinomycin. After culture for 12 to 20 hours, positive single colonies were selected to extract plasmids for sequencing, to obtain a correct pCDF-accBC plasmid.

Fifth step: A pCDF-accBC plasmid was used as a template to be amplified by using primers P2-R/P2-F to obtain a vector backbone fragment, with a fragment size of 5557 bp, containing a T7 promoter, an accBC gene, an Ori sequence, and a spectinomycin resistance gene Smr, and referred as fragment S, wherein a sequence of P2-R was set forth in SEQ ID NO: 64, a sequence of P2-F was set forth in SEQ ID NO: 65, and a sequence of the fragment S was set forth in SEQ ID NO: 66.

Sixth step: a Corynebacterium glutamicum genome was used as a template to be amplified by using primers accD-up/accD-down to obtain an accD coding gene, with a gene fragment size of 1683 bp, containing an accD coding sequence, and referred as fragment T (SEQ ID NO: 67), wherein a sequence of accD-up was set forth in SEQ ID NO: 68; a sequence of accD-down was set forth in SEQ ID NO: 69.

Seventh step: the templates were removed from the fragment S and the fragment T by using an endonuclease DpnI; after electrophoresis, the same was cleaned by using a PCR purification kit; the fragment S and the fragment T were taken, and 2× Seamless Cloning Mix was added for reaction for 30 to 60 minutes.

Eighth step: a reaction product in the seventh step was taken to be added into Trans-T1 competent cells, subjected to ice bath and heat shock, and placed on ice for 2 to 5 minutes; an SOC medium was added for incubation; centrifugation was carried out to remove a supernatant. After even mixing, the product was coated to an LB plate containing spectinomycin. After culture for 12 to 20 hours, positive single colonies were selected to extract plasmids for sequencing, to obtain a correct pCDF-ACC plasmid.

Obtaining a strain: 50 ng of the plasmid pCDF-ACC was taken to be added into 50 μL of MG1655 (DE3) competent cells (purchased from Shanghai Yuchun Biology Science and Technology Co., Ltd.), and ice bath was carried out for 30 minutes. Heat shock was carried out at 42° C. for 60 seconds, and the same was immediately placed on ice for 2 minutes. 500 μL of an SOC medium (purchased from Shanghai Sangon Biotech Co., Ltd.) was added, and incubation was carried out at 220 rpm and 37° C. for 1 hour. Centrifugation was carried out to remove a supernatant, left over approximately 50 μL. After mixing well, all were coated to an LB plate containing spectinomycin (a final concentration of 50 μg/mL). After overnight culture, positive colonies were obtained, to obtain engineered bacteria MG1655 (DE3), pCDF-ACC (strain N).

Monoclones of the MG1655 (DE3), pCDF-bauA-mcrC (strain L), monoclones of the MG1655 (DE3), pCDF-duet (strain M) and monoclones of the MG1655 (DE3), pCDF-ACC (strain N) were selected respectively to be inoculated into a 15 mL test tube containing 5 mL of an MOPS medium, the medium containing spectinomycin (a final concentration of 50 μg/mL). Culture was carried out at 220 r and 37° C. until the OD₅₅₀ is around 1.5 to 1.8 to be used as a seed solution.

The seed solution was inoculated at a ratio of initial OD₅₅₀=0.1 into an MOPS medium (containing 20 μg/L glucose, 0.1 mM IPTG, and chloramphenicol and ampicillin with a final concentration of 50 μg/mL). After inoculation was completed, shaking for even mixing was carried out. 200 μL of the same was pipetted to a 96-well plate. By using a continuous kinetic assay method, OD₅₅₀ was determined every 10 minutes with a microplate reader (Biotek), to determine the growth curve of the strain. The specific growth rate μ=ln(OD550_{T}−OD550_{O})/T can be obtained by calculation of the exponential growth phase OD550_{T}, cultivation time T, and initial OD550_{O}.

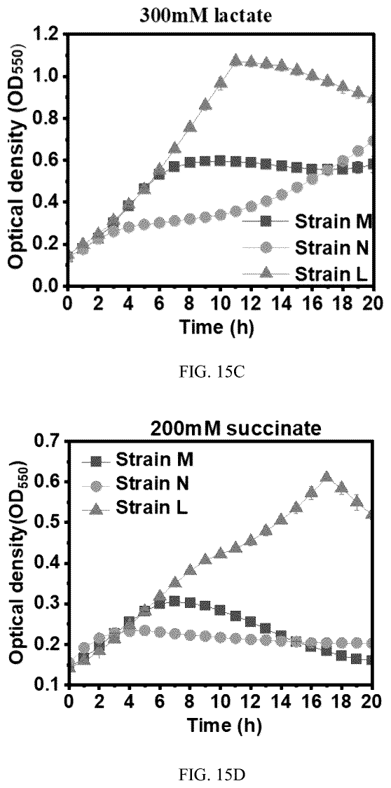

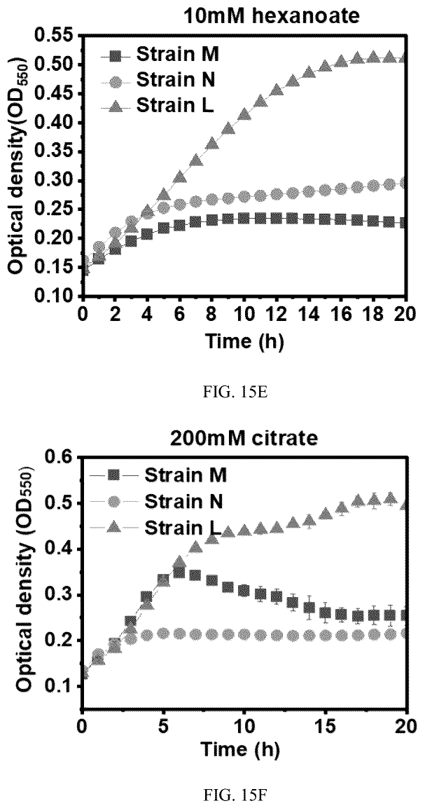

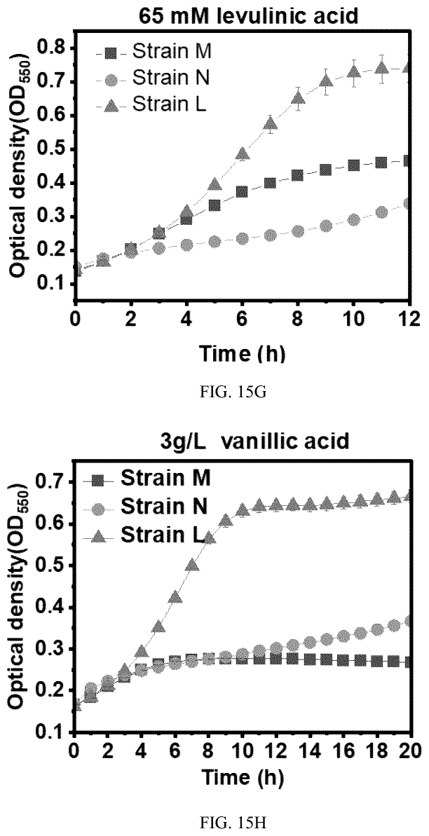

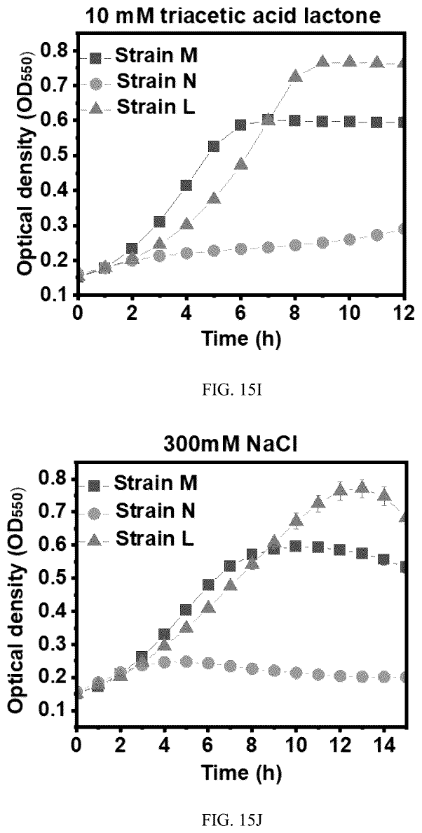

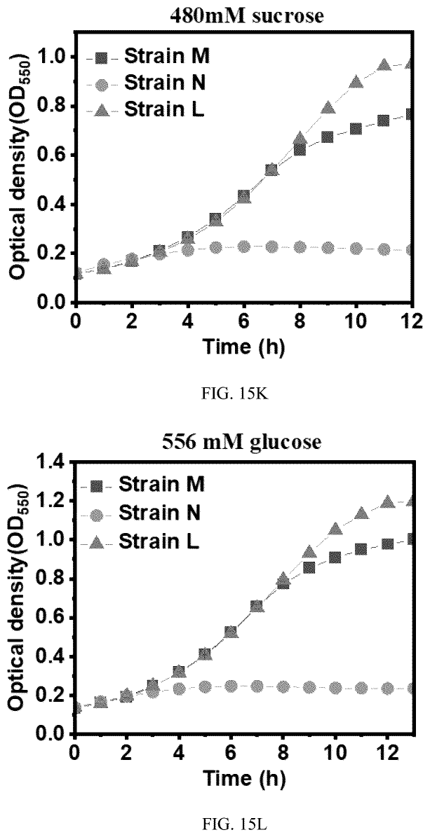

Results show that: compared with the reference strain M, the specific growth rate (μ, h⁻¹) of ACC overexpressed E. coli strain N significant decreases: from 0.50 h⁻¹ to 0.25 h⁻¹, with a decrease of 50% (FIGS. 15A-15L). Compared with ACC, the specific growth rate of NCM strain overexpressed E. coli strain L slightly decreases (=0.41 h⁻¹), indicating that overexpression of the NCM pathway does not lead to severe cytotoxicity (FIGS. 15A-15L).

### Embodiment 14 the New Pathway Improves Tolerance of Strains

### Tolerance Test Method

Seed culture: Monoclones of the MG1655 (DE3), pCDF-bauA-mcrC (strain L), monoclones of the MG1655 (DE3), pCDF-duet (strain M) and monoclones of the MG1655 (DE3), pCDF-ACC (strain N) were selected respectively to be inoculated into a 15 mL test tube containing 5 mL of an MOPS medium, the medium containing spectinomycin (a final concentration of 50 μg/mL). Culture was carried out at 220 r and 37° C. until the OD₅₅₀ is around 1.5 to 1.8 to be used as a seed solution.

Medium used for tolerance testing: IPTG (a final concentration of 0.1 mM) and spectinomycin (a final concentration of 50 μg/mL) were added into an MOPS medium (containing 20 μg/L glucose); then, inhibitors were added, and the selected inhibitors and final concentrations thereof were formic acid (100 mM), acetic acid (50 mM), caproic acid (10 mM), lactic acid (500 mM), succinic acid (200 mM), glucose (100 μg/L), sucrose (480 mM), octanoic acid (10 mM), citric acid (200 mM), vanillic acid (3 μg/L), and triacetic acid lactone (TAL, 1.25 μg/L).

### 2. Tolerance Test Method

The seed solution was inoculated at a ratio of initial OD₅₅₀=0.1 into a medium for tolerance test. After inoculation was completed, shaking for even mixing was carried out. 200 μL of the same was pipetted to a 96-well plate. By using a continuous kinetic assay method, OD₅₅₀ was determined every 10 minutes with a microplate reader (Biotek), to determine the growth curve of the strain. The specific growth rate μ=ln(OD550_{T}−OD550_{O})/T can be obtained by calculation of the exponential growth phase OD550_{T}, cultivation time T, and initial OD550_{O}.

Results show that: overexpression of the NCM pathway increases the tolerance of E Coli to various toxic chemicals, such as organic acids. The tolerance of NCM pathway overexpressed E Coli to succinic acid (200 mM succinic acid [23.6 μg/L]) is significantly improved, and compared with the reference strain (OD₅₅₀=0.30), the biomass (OD₅₅₀=0.60) increases by 100%. In contrast, the growth ability of ACC pathway overexpressed E. coli under this concentration condition significantly decreases, or is even completely inhibited (OD₅₅₀=0.20) (FIGS. 15A-15L). Similar phenomena are observed when the strains are attacked by other organic acids, including lactic acid, acetic acid, caproic acid, and citric acid (FIGS. 15A-15L).

In addition to organic acids, overexpression of the NCM pathway increases the tolerance of E. coli to polyketides (FIGS. 15A-15L): by means of taking the simplest polyketide-triacetic acid lactone (10 mM, [1.26 μg/L]) as an example, the biomass of the NCM E. coli strain (OD₅₅₀=0.81) increases by 30% compared to the reference E. coli strain (OD₅₅₀=0.62), and also increases by 180% compared to the ACC overexpressed E. coli strain (OD₅₅₀=0.29).

Moreover, overexpression of the NCM pathway also increases the tolerance of E. coli to adverse environmental stress (FIGS. 15A-15L). At a higher osmotic pressure (300 mM NaCl [˜17.5 μg/L]), the biomass of the NCM pathway overexpressed E. coli strain (OD₅₅₀=0.76) increases by 29% and 214%, respectively, compared to the reference strain (OD₅₅₀=0.59) and the ACC overexpressed strain (OD₅₅₀=0.24). In the presence of high-concentration sugar (480 mM sucrose [−164 μg/L]), the biomass of NCM pathway overexpressed E. coli strain L (OD₅₅₀=0.96) increases by 26% and 320%, respectively, compared to the reference strain M (OD₅₅₀=0.77) and the ACC strain N (OD₅₅₀=0.23).

The specific embodiments of the present invention are described above. It needs to be understood that the present invention is not limited to the specific implementations mentioned above. Those skilled in the art can make various variations or modifications within the scope of the claims, which does not affect the substantive content of the present invention.

What is claimed is:

1. An artificial synthesis method for malonyl-coenzyme A (CoA), comprising the following steps:
S1, forming 3-oxopropanoate and a compound represented by a formula (2) by β-alanine and a compound represented by a formula (1) under a catalysis of an aminotransferase, 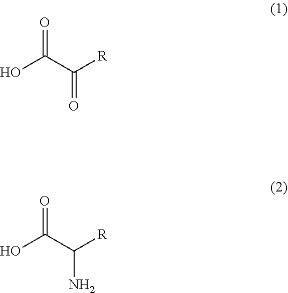
wherein R is selected from H, alkanes, alcohols, and alkanoic acids; and
S2, forming the malonyl-CoA and 2[H] by the 3-oxopropanoate, CoA, and an electron acceptor by transferring electrons of the 3-oxopropanoate to the electron acceptor under a catalysis of an oxidoreductase.

2. The artificial synthesis method for the malonyl-CoA according to claim 1, wherein the compound represented by the formula (1) comprises α-ketonic acid.

3. The artificial synthesis method for the malonyl-CoA according to claim 2, wherein the α-ketonic acid is at least one selected from the group consisting of pyruvic acid, oxaloacetic acid, and α-ketoglutaric acid.

4. The artificial synthesis method for the malonyl-CoA according to claim 1, wherein the compound represented by the formula (2) comprises α-amino acid.

5. The artificial synthesis method for the malonyl-CoA according to claim 4, wherein the α-amino acid comprises α-alanine, aspartic acid, or glutamic acid.

6. The artificial synthesis method for the malonyl-CoA according to claim 1, wherein in the step S1, the aminotransferase comprises at least one of BauA from Pseudomonas aeruginosa, AptA from Acinetobacter pittii, CCNA_03245 from Caulobacter vibrioides, PydD2 from Bacillus megaterium, YhxA from Bacillus cereus, PydD from Ureibacillus massiliensis, and Pyd4 from Saccharomyces kluyveri.

7. The artificial synthesis method for the malonyl-CoA according to claim 6, wherein
the amino acid sequence of the BauA is set forth in SEQ ID NO: 7;
the amino acid sequence of the AptA is set forth in SEQ ID NO: 8;
the amino acid sequence of the CCNA_03245 is set forth in SEQ ID NO: 9;
the amino acid sequence of the PydD2 is set forth in SEQ ID NO: 10;
the amino acid sequence of the YhxA is set forth in SEQ ID NO: 11;
the amino acid sequence of the PydD is set forth in SEQ ID NO: 71; and
the amino acid sequence of the Pyd4 is set forth in SEQ ID NO: 72.

8. The artificial synthesis method for the malonyl-CoA according to claim 1, wherein the oxidoreductase comprises at least one of MCR-C from Chloroflexus aurantiacu, StMCR from Sulfurisphaera tokodaii, Msed_0709 from Metallosphaera sedula, PnSCR from Pyrobaculum neutrophilum, and SucD from Clostridium kluyveri.

9. The artificial synthesis method for the malonyl-CoA according to claim 8, wherein
the amino acid sequence of the MCR-C is set forth in SEQ ID NO: 70;
the amino acid sequence of the StMCR is set forth in SEQ ID NO: 12;
the amino acid sequence of the Msed_0709 is set forth in SEQ ID NO: 13;
the amino acid sequence of the PnSCR is set forth in SEQ ID NO: 14; and
the amino acid sequence of the SucD is set forth in SEQ ID NO: 15.

10. The artificial synthesis method for the malonyl-CoA according to claim 9, wherein a preparation method for the MCR-C comprises the following steps:
1) constructing a recombinant plasmid pCDF-P1-mcrC based on the amino acid sequence of the MCR-C;
2) transferring the recombinant plasmid pCDF-P1-mcrC into E. coli BL21 DE3 competent cells to obtain a positive monoclonal colony;
3) cultivating the positive monoclonal colony under an induction with an inducer to obtain a culture product;
4) centrifuging and purifying the culture product in the step 3) to obtain a bacterial cell, adding a nondenaturing lysis buffer to resuspend the bacterial cell, adding a lysozyme for even mixing to obtain a bacterial suspension, cooling and sonicating the bacterial suspension, and lysing bacteria to obtain a treated suspension; centrifuging the treated suspension, and collecting a supernatant to prepare a bacterial lysis solution; and
5) carrying out a chromatography on the bacterial lysis solution prepared in the step 4) to prepare a purified MCR-C protein sample.

11. The artificial synthesis method for the malonyl-CoA according to claim 10, wherein a construction method for the recombinant plasmid pCDF-P1-mcrC comprises the following steps:
1) using a pCDF-duet plasmid containing 6×His tag as a first template to be amplified by using primers P1-R/P1-F to obtain a pCDF vector backbone fragment, wherein the pCDF vector backbone fragment contains a T7 promoter, an Ori sequence, and a spectinomycin resistance gene Smr, and the pCDF vector backbone fragment is referred as a first fragment, wherein the sequence of P1-R is set forth in SEQ ID NO: 1, the sequence of P1-F is set forth in SEQ ID NO: 2, and the sequence of the first fragment is set forth in SEQ ID NO: 3;
2) using a pMCR-C plasmid as a second template to be amplified by using primers MCR-C-pCDF-P1-up/MCR-C-pCDF-P1-down to obtain an MCR-C coding gene, wherein the MCR-C coding gene is referred as a second fragment, wherein the sequence of MCR-C-pCDF-P1-up is set forth in SEQ ID NO: 4, the sequence of MCR-C-pCDF-P1-down is set forth in SEQ ID NO: 5, and the sequence of the second fragment is set forth in SEQ ID NO: 6;
3) removing the first template and the second template from the first fragment and the second fragment respectively by using an endonuclease DpnI, and after an electrophoresis, cleaning the first fragment and the second fragment by using a PCR purification kit, taking the first fragment and the second fragment after a template removal, and adding 2× Seamless Cloning Mix for a reaction to obtain a reaction product; and
4) taking the reaction product in the step 3) to be transferred into Trans-T1 competent cells, selecting a positive single colony, and carrying out a colony PCR validation, with primers P1-YZ-up/MCR-C-YZ-down, to obtain the recombinant plasmid pCDF-P1-mcrC, wherein the sequence of P1-YZ-up is set forth in SEQ ID NO: 54, and the sequence of MCR-C-YZ-down is set forth in SEQ ID NO: 55.

12. The artificial synthesis method for the malonyl-CoA according to claim 7, wherein a preparation method for the BauA comprises the following steps:
step 1, constructing a recombinant plasmid pCDF-P1-bauA based on the amino acid sequence of the BauA;
step 2, transferring the recombinant plasmid pCDF-P1-bauA into E. coli BL21 DE3 competent cells to obtain a positive monoclonal colony;
step 3, cultivating the positive monoclonal colony in the step 2 under an induction with an inducer to obtain a culture product;
step 4, centrifuging and purifying the culture product in the step 3 to obtain a bacterial cell, adding a nondenaturing lysis buffer to resuspend the bacterial cell, adding a lysozyme for even mixing to obtain a bacterial suspension, cooling and sonicating the bacterial suspension, and lysing bacteria to obtain a treated suspension; centrifuging the treated suspension, and collecting a supernatant to prepare a bacterial lysis solution; and
step 5, carrying out a chromatography on the bacterial lysis solution prepared in the step 4 to prepare a purified BauA protein sample.

13. A recombinant plasmid comprising BauA and MCR-C, wherein the amino acid sequence of the BauA is set forth in SEQ ID NO: 7; and the amino acid sequence of the MCR-C is set forth in SEQ ID NO: 70.

14. A method for synthesizing a fatty acid with the recombinant plasmid according to claim 13, wherein
the recombinant plasmid uses pCDF as a vector and the recombinant plasmid is named pCDF-bauA-mcrC; and the method comprises the following steps:
transferring the recombinant plasmid pCDF-bauA-mcrC and a thioesterase expression plasmid into E. coli competent cells to obtain a positive clonal strain, subjecting the positive clonal strain to a fermentation and a cultivation to obtain the fatty acid.

15. The method according to claim 14, wherein the fatty acid comprises octanoic acid, decanoic acid, lauric acid, tetradecanoic acid, or hexadecanoic acid.

16. A method for synthesizing a polyketide with the recombinant plasmid according to claim 13, wherein the recombinant plasmid uses pCDF as a vector and the recombinant plasmid is named pCDF-bauA-mcrC; and the method comprises the following steps: transferring the recombinant plasmid pCDF-bauA-mcrC and a polyketide synthase coding gene into first E. coli competent cells to obtain a first positive clonal strain, subjecting the first positive clonal strain to a first fermentation and a first cultivation to obtain the polyketide.

17. The method according to claim 16, wherein the polyketide comprises phloroglucinol, flaviolin, and pentadecaheptaene.

18. The method according to claim 17, wherein a synthesis method for the phloroglucinol comprises the following steps: transferring the recombinant plasmid pCDF-bauA-mcrC and a plasmid pCum-phlD into second E. coli competent cells to obtain a second positive clonal strain, subjecting the second positive clonal strain to a second fermentation and a second cultivation to obtain the phloroglucinol.

19. The method according to claim 17, wherein a synthesis method for the flaviolin comprises the following steps: transferring the recombinant plasmid pCDF-bauA-mcrC and a plasmid pCum-rppA into second E. coli competent cells to obtain a second positive clonal strain, subjecting the second positive clonal strain to a second fermentation and a second cultivation to obtain the flaviolin.

20. The method according to claim 17, wherein a synthesis method for the pentadecaheptaene comprises the following steps: transferring the recombinant plasmid pCDF-bauA-mcrC and a plasmid pQL1 into second E. coli competent cells to obtain a second positive clonal strain, subjecting the second positive clonal strain to a second fermentation and a second cultivation to obtain the pentadecaheptaene.

21. A method for synthesizing natamycin or spinosad with the recombinant plasmid according to claim 13, wherein the recombinant plasmid uses SET152 as a vector to express the BauA and the MCR-C respectively by using strong promoters KasOP and SPL42, and the recombinant plasmid is named SET152-KasOP-bauA-SPL42-mcrC;
the method for synthesizing the natamycin comprises the following steps:
transferring the recombinant plasmid SET152-KasOP-bauA-SPL42-mcrC into Streptomyces gilvosporeus to obtain a first positive clonal strain, subjecting the first positive clonal strain to a first fermentation and a first cultivation to obtain the natamycin; and
the method for synthesizing the spinosad comprises the following steps:
transferring the recombinant plasmid SET152-KasOP-bauA-SPL42-mcrC into Saccharopolyspora spinosa to obtain a second positive clonal strain, subjecting the second positive clonal strain to a second fermentation and a second cultivation to obtain the spinosad.


SEQUENCE LISTING (72 sequences)

SEQ ID NO: 1 - DNA, 18 bp, synthetic construct
  misc_feature 1..18: note = The sequence is synthetized.
  source 1..18: mol_type = other DNA
cggatcctggctgtggtg

SEQ ID NO: 2 - DNA, 17 bp, synthetic construct
  misc_feature 1..17: note = The sequence is synthetized.
  source 1..17: mol_type = other DNA
aattcgagctcggcgcg

SEQ ID NO: 3 - DNA, 3781 bp, synthetic construct
  misc_feature 1..3781: note = The sequence is synthetized.
  source 1..3781: mol_type = other DNA
aattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggcagcagccatcaccatcatcaccacagccaggatccg

SEQ ID NO: 4 - DNA, 35 bp, synthetic construct
  misc_feature 1..35: note = The sequence is synthetized.
  source 1..35: mol_type = other DNA
caccacagccaggatccgagcgccaccaccggcgc

SEQ ID NO: 5 - DNA, 37 bp, synthetic construct
  misc_feature 1..37: note = The sequence is synthetized.
  source 1..37: mol_type = other DNA
cgcgccgagctcgaattttacacggtaatcgcccgtc

SEQ ID NO: 6 - DNA, 2048 bp, synthetic construct
  misc_feature 1..2048: note = The sequence is synthetized.
  source 1..2048: mol_type = other DNA
caccacagccaggatccgagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcgtggtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgctggattgccctgagtgatggtgcccgtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaaaattcgagctcggcgcg

SEQ ID NO: 7 - protein, 448 aa, synthetic construct
  REGION 1..448: note = The sequence is synthetized.
MNQPLNVAPPVSSELNLRAHWMPFSANRNFQKDPRIIVAAEGSWLTDDKGRKVYDSLSGLWTCGAGHSRKEIQEAVARQLGTLDYSPGFQYGHPLSFQLAEKIAGLLPGELNHVFFTGSGSECADTSIKMARAYWRLKGQPQKTKLIGRARGYHGVNVAGTSLGGIGGNRKMFGQLMDVDHLPHTLQPGMAFTRGMAQTGGVELANELLKLIELHDASNIAAVIVEPMSGSAGVLVPPVGYLQRLREICDQHNILLIFDEVITAFGRLGTYSGAEYFGVTPDLMNVAKQVTNGAVPMGAVIASSEIYDTFMNQALPEHAVEFSHGYTYSAHPVACAAGLAALDILARDNLVQQSAELAPHFEKGLHGLQGAKNVIDIRNCGLAGAIQIAPRDGDPTVRPFEAGMKLWQQGFYVRFGGDTLQFGPTFNARPEELDRLFDAVGEALNGIA

SEQ ID NO: 8 - protein, 460 aa, synthetic construct
  REGION 1..460: note = The sequence is synthetized.
MVMFDTDKFSDSEHTLDAVQPNNNMYINYQAHWMPFSANRNFAKDPRMIVGAKGSYLIDDSGREIYDSLSGLWTCGAGHTLPEIQQAVSAQLGQLDYSPAFQFGHPLSFKLADKIVQHMPEKLQHVFFTNSGSESADTAIKMARAYWRIKGKPSKTKLIGRARGYHGVNVAGTSLGGIGGNRKMFGQLMDVDHLPHTLQPNLTFTKGCAETGGVELANEMLKLIELHDASNIAAVIVEPISGSAGCIVPPTGYLQRLREICDQHDILLIFDEVITGFGRLGTWTGAEYFGVTPDILNFAKQVTNGAIPLGGVVASHEIYSAFMQQELPEHAIEFTHGYTYSAHPVACAAALAALEVLEKKNLLAQSAALAPSFEKMLHGLKGAPHVLDIRNCGLIGALQLAPRDGDATIRGFELGMKLWKEGFYVRFGGDTLQFGPMFNGTEADIDRLMNAVGDALYQVN

SEQ ID NO: 9 - protein, 442 aa, synthetic construct
  REGION 1..442: note = The sequence is synthetized.
MLPMPDFGANDLDAFWMPFTPNRRFKRHPRMLSSASGMWYRTPESREVLDATSGLWCVNAGHDRPKIREAIQKQAAEMDYAPCFNMGHPLAFQFASRLAQITPKGLDRIFFTNSGSESVDTALKIALAYHRARGKGTKTRLIGRERGYHGVGFGGISVGGIPKNRMYFGSLLTGVDHLPHTHGLPGNTCAKGQPENGAHLADDLERIVALHDASNIAAVIVEPVAGSTGVLIPPKGYLERLRAICDKHDILLIFDEVITGFGRVGAPFAAERFGVTPDLICMAKGLTNAAVPCGAVAASGKIYDAMMDGADAPIELFHGYTYSAHPLACAAGLATLETYREDDLFARAAGLEGYWQDAMHSLADARHVVDVRNLGLVAGIELEPRPGAPTARAMEVFETCFDEGLLIRVTGDIIALSPPLILEKDHIDRMVETIRRVLGQVD

SEQ ID NO: 10 - protein, 452 aa, synthetic construct
  REGION 1..452: note = The sequence is synthetized.
MVQVSQNEKDLRAKDEQYVWHGMKPYSPDATMVATKAKGAWITDEQGNRYLDAMAGLWCVNTGYGRDELALAAYEQLKELAYFPLTQSHVPAIKLAEKLNEMLGDDYVIFFSNSGSEANETAFKIVRQYHQQKGEHSRYKFISRYRGYHGNSMGALAATGQAQRKYKYEPLAPGFIHVAPPDSYRGQDDYDAPKEQLQSVKDIDNVMTWELSETIAAVIMEPIITGGGILLPPQNYMKGVKETCEKHGALLIADEVICGFGRTGEAFGFMNYDVKPDIITMAKGITSAYLPLSATAVKKEIYEAFKDSDQYEYFRHVNTFGGSPAACALALKNLEIMERENLFDRSKQLGAYVLSELTPLIDSHPYVGDVRGKGLLIGIELVADKQTKEPLDVNLVNEVVAKCKSKGVIIGKNGATVAGYNNVLTLSPPLNIEESDIELLLATITYALDSIK

SEQ ID NO: 11 - protein, 451 aa, synthetic construct
  REGION 1..451: note = The sequence is synthetized.
MELMIVQVTEQTQSLKKTDEKYLWHAMRGAAPSPTNLIITKAEGAWVTDIDGNRYLDGMSGLWCVNVGYGRKELARAAFEQLEEMPYFPLTQSHVPAIKLAEKLNEWLDDEYVIFFSNSGSEANETAFKIARQYHQQKGDHGRYKFISRYRAYHGNSMGALAATGQAQRKYKYEPLGQGFLHVAPPDTYRNPEDVHTLASAEEIDRVMTWELSQTVAGVIMEPIITGGGILMPPDGYMEKVKEICEKHGALLICDEVICGFGRTGKPFGFMNYGVKPDIITMAKGITSAYLPLSATAVRREVYEAFVGSDDYDRFRHVNTFGGNPAACALALKNLEIMENEKLIERSKELGERLLYELEDVKEHPNVGDVRGKGLLLGIELVEDKQTKEPASIEKMNKVINACKEKGLIIGKNGDTVAGYNNILQLAPPLSITEEDFTFIVKTMKECLAQL

SEQ ID NO: 12 - protein, 359 aa, synthetic construct
  REGION 1..359: note = The sequence is synthetized.
MILMRRTLKAAILGATGLVGIEYVRMLSNHPYIKPAYLAGKGSVGKPYGEVVRWQTVGQVPKEIADMEIKPTDPKLMDDVDIIFSPLPQGAAGPVEEQFAKEGFPVISNSPDHRFDPDVPLLVPELNPHTISLIDEQRKRREWKGFIVTTPLCTAQGAAIPLGAIFKDYKMDGAFITTIQSLSGAGYPGIPSLDVVDNILPLGDGYDAKTIKEIFRILSEVKRNVDEPKLEDVSLAATTHRIATIHGHYEVLYVSFKEETAAEKVKETLENFRGEPQDLKLPTAPSKPIIVMNEDTRPQVYFDRWAGDIPGMSVVVGRLKQVNKRMIRLVSLIHNTVRGAAGGGILAAELLVEKGYIEK

SEQ ID NO: 13 - protein, 357 aa, synthetic construct
  REGION 1..357: note = The sequence is synthetized.
MRRTLKAAILGATGLVGIEYVRMLADHPYIKPTYLAGKGSVGKPYGEIVRWQTVGNVPKEVANQEVKPTDPKLMDDVDIIFSPLPQGAAGPVEEQFAKLGFNVISNSPDHRFDMDVPMIIPEVNPHTVTLIDEQRKRRDWKGFIVTTPLCTAQGAAIPLTPIYQNFKMSGVMITTMQSLSGAGYPGIASLDIVDNALPLGDGYDAKTVKEITRILSEVKRNVQEPGVNEITLDATTHRIATIHGHYEVAYVTFKEDTDVRKVMESMESFKGEPQDLKLPTAPEKPIIVTTQDARPQVFFDRWAGNPPGMSVVVGRLKQVNPRTIRFVSLIHNTVRGAAGGGVLTAELLVEKGYIDKR

SEQ ID NO: 14 - protein, 469 aa, synthetic construct
  REGION 1..469: note = The sequence is synthetized.
MTNLVVVNPATEEAIAELPQATREDVRRAIDAAWDAFASWSALPLRKRTRVLLKTAELAEAAREDLLKTLVAESGKPIRDAEAEITRAIDIFRSSAEEAKLILEGSAPRVDAYEYPIGNENRLVVAVREPVGVVGGALSYNNPASTFAHKVAPVIAAGNTVVVKPSSYTPLTALKFLEIMKRAGVPEGVVNVVVGSGEEVFDELIQSDKVAGINFTGSTAVGLQVAAKAASRGKKFMIAPGGSDPAVVFKDADLDAAARIVARARYENAGQNCNATKRVFVEREVYPRFVELLLGYVKAIRVGDPMDYSTDMGPLISEKIVKAMDGVVKDALEKGAKLAAGGRRMNRRGYFYEPTVLLFDGDAEAKALREEVFGPVLPVVPFEGEEEAVRLANATQYGLQSAVFTSDYRKALRVARGIKAGAVMINESTRVRFDALPYGGVKMSGFGWREGVRSTMIYYTEPKFLVFGL

SEQ ID NO: 15 - protein, 453 aa, synthetic construct
  REGION 1..453: note = The sequence is synthetized.
MSNEVSIKELIEKAKVAQKKLEAYSQEQVDVLVKALGKVVYDNAEMFAKEAVEETEMGVYEDKVAKCHLKSGAIWNHIKDKKTVGIIKEEPERALVYVAKPKGVVAATTPITNPVVTPMCNAMAAIKGRNTIIVAPHPKAKKVSAHTVELMNAELKKLGAPENIIQIVEAPSREAAKELMESADVVIATGGAGRVKAAYSSGRPAYGVGPGNSQVIVDKGYDYNKAAQDIITGRKYDNGIICSSEQSVIAPAEDYDKVIAAFVENGAFYVEDEETVEKFRSTLFKDGKINSKIIGKSVQIIADLAGVKVPEGTKVIVLKGKGAGEKDVLCKEKMCPVLVALKYDTFEEAVEIAMANYMYEGAGHTAGIHSDNDENIRYAGTVLPISRLVVNQPATTAGGSFNNGFNPTTTLGCGSWGRNSISENLTYEHLINVSRIGYFNKEAKVPSYEEIWG

SEQ ID NO: 16 - DNA, 660 bp, synthetic construct
  misc_feature 1..660: note = The sequence is synthetized.
  source 1..660: mol_type = other DNA
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 17 - DNA, 4107 bp, synthetic construct
  misc_feature 1..4107: note = The sequence is synthetized.
  source 1..4107: mol_type = other DNA
atggacaaaaaatactctatcggtctggctatcggtactaactctgttggttgggctgttatcaccgacgaatacaaagttccgtctaaaaaattcaaagttctgggtaacaccgaccgtcactctatcaaaaaaaacctgatcggtgctctgctgttcgactctggtgaaaccgctgaagctacccgtctgaaacgtaccgctcgtcgtcgttacacccgtcgtaaaaaccgtatctgctacctgcaagaaatcttctctaacgaaatggctaaagttgacgactctttcttccaccgtctcgaagaatcgttcctggttgaagaggacaaaaaacacgaacgtcacccgatcttcggtaacatcgttgacgaagttgcttaccacgaaaaatacccgaccatctaccacctgcgtaaaaaactggttgactctaccgacaaagctgacctgcgtctgatctacctggctctggctcacatgatcaaattccgtggtcacttcctgatcgaaggtgacctgaacccggacaactctgacgttgacaaactgttcatccagctggttcagacctacaaccagctgttcgaagaaaacccgatcaacgcttctggtgttgacgctaaagctatcctgtctgctcgtctgtctaaatctcgtcgtctggaaaacctgatcgctcagctgccgggtgaaaaaaaaaacggtctgttcggtaacctgatcgctctgtctctgggtctgaccccgaacttcaaatctaacttcgacctggctgaagacgctaaactgcaactgtctaaagacacctacgacgacgacctggacaacctgctggctcagatcggtgaccagtacgctgacctgttcctggctgctaaaaacctgtctgacgctatcctgctgtctgacatcctgcgtgttaacaccgaaatcaccaaagctccgctgtctgcttctatgatcaaacgttacgacgaacaccaccaggacctgaccctgctgaaagctctggttcgtcagcagctgccggaaaaatacaaagaaatcttcttcgaccagtctaaaaacggttacgctggttacatcgacggtggtgcttctcaggaagaattttacaaattcatcaaaccgatcctggaaaaaatggacggtactgaagaactgctggttaaactgaaccgtgaagacctgctgcgtaaacagcgtaccttcgacaacggttctatcccgcaccagatccacctgggtgaactgcacgctatcctgcgtcgtcaggaagacttctacccgttcctgaaagacaaccgtgaaaaaatcgaaaaaatcctgaccttccgtatcccgtactacgttggtccgctggctcgtggtaactctcgtttcgcttggatgacccgtaaatctgaagaaaccatcaccccgtggaacttcgaagaagttgttgacaaaggtgcttctgctcagtctttcatcgaacgtatgaccaacttcgacaaaaacctgccgaacgaaaaagttctgccgaaacactctctgctgtacgaatacttcaccgtttacaacgaactgaccaaagttaaatacgttaccgaaggtatgcgtaaaccggctttcctgtctggtgaacagaaaaaagctatcgttgacctgctgttcaaaaccaaccgtaaagttaccgttaaacagctgaaagaagactacttcaaaaaaatcgaatgcttcgactctgttgaaatctctggtgttgaagaccgtttcaacgcttctctgggtacttaccacgacctgctgaaaatcatcaaagacaaagacttcctggacaacgaagaaaacgaagacatcctggaagacatcgttctgaccctgaccctgttcgaagaccgtgaaatgatcgaagaacgtctgaaaacctacgctcacctgttcgacgacaaagttatgaaacagctgaaacgtcgtcgttacaccggttggggtcgtctgtctcgtaaactgatcaacggtatccgtgacaaacagtctggtaaaaccatcctggacttcctgaaatctgacggtttcgctaaccgtaacttcatgcagctgatccacgacgactctctgaccttcaaagaagacatccagaaggcgcaggtaagcggtcagggtgactctctgcacgaacacatcgctaacctggctggttctccggctatcaaaaaaggtatcctgcaaaccgttaaagttgttgacgaactggttaaagttatgggtcgtcacaaaccggaaaacatcgttatcgaaatggctcgtgaaaaccagaccacccagaaaggtcagaaaaactctcgtgaacgtatgaaacgtatcgaagaaggtatcaaagaactgggttctcagatcctgaaagaacacccggttgaaaacacccagctgcaaaacgaaaaactgtacctgtactacctgcaaaacggtcgtgacatgtacgttgaccaggaactggacatcaaccgtctgtctgactacgacgttgacgctatcgttccgcagtctttcctgaaagacgactctatcgacaacaaagttctgacccgttctgacaaaaaccgtggtaaatctgacaacgttccgtctgaagaagttgttaaaaaaatgaaaaactactggcgtcagctgctgaacgctaaactgatcacccagcgtaaattcgacaacctgaccaaagctgaacgtggtggtctgtctgaactggacaaagctggtttcatcaaacgtcagctggttgaaacccgtcagatcaccaaacacgttgctcagatcctggactctcgtatgaacaccaaatacgacgaaaacgacaaactgatccgtgaagttaaagttatcaccctgaaatctaaactggtttctgacttccgtaaagacttccagttctacaaagttcgtgaaatcaacaactaccaccacgctcacgacgcttacctgaacgctgttgttggtactgctctgatcaaaaaatacccgaaactggagtcggaatttgtgtacggggactacaaagtttacgacgttcgtaaaatgatcgctaaatctgaacaggaaatcggtaaagctaccgctaaatacttcttctactctaacatcatgaacttcttcaaaaccgaaatcaccctggctaacggtgaaatccgtaaacgtccgctgatcgaaaccaacggtgaaaccggcgagatcgtctgggacaaaggccgtgacttcgctaccgttcgtaaagttctgtctatgccgcaggttaacatcgttaaaaaaaccgaagttcagaccggtggtttctctaaagaatctatcctgccgaaacgtaactctgacaaactgatcgctcgtaaaaaagactgggacccaaaaaaatacggtggcttcgactctccgactgttgcttactctgttctggttgttgctaaagttgaaaaaggtaaatctaaaaaactgaaatctgttaaagaactgctgggtatcaccatcatggaacgttcttctttcgaaaaaaacccgatcgacttcctggaagctaaaggttacaaagaagttaaaaaagacctgatcatcaaactgccgaaatactctctgttcgaactggaaaacggtcgtaaacgtatgctggcttctgctggtgaactgcaaaaaggtaacgaactggctctgccgtctaaatacgttaacttcctgtacctggcttctcactacgaaaaactgaaaggttctccggaagacaacgaacagaaacagctgttcgttgaacagcacaaacactacctggacgaaatcatcgaacagatcagtgaattttctaaacgtgttatcctggctgacgctaacctggacaaagttctgtctgcttacaacaaacaccgtgacaaaccgatccgtgaacaggctgaaaacatcatccacctgttcaccctgaccaacctcggtgctccagctgcgttcaaatacttcgacaccaccatcgaccgtaaacgttacacctctaccaaagaagttctggacgctaccctgatccaccagtctatcaccggtctgtacgaaacccgtatcgacctgtctcagctgggtggtgactaa

SEQ ID NO: 18 - DNA, 8184 bp, synthetic construct
  misc_feature 1..8184: note = The sequence is synthetized.
  source 1..8184: mol_type = other DNA
ggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgctgcagacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggacaaaaaatactctatcggtctggctatcggtactaactctgttggttgggctgttatcaccgacgaatacaaagttccgtctaaaaaattcaaagttctgggtaacaccgaccgtcactctatcaaaaaaaacctgatcggtgctctgctgttcgactctggtgaaaccgctgaagctacccgtctgaaacgtaccgctcgtcgtcgttacacccgtcgtaaaaaccgtatctgctacctgcaagaaatcttctctaacgaaatggctaaagttgacgactctttcttccaccgtctcgaagaatcgttcctggttgaagaggacaaaaaacacgaacgtcacccgatcttcggtaacatcgttgacgaagttgcttaccacgaaaaatacccgaccatctaccacctgcgtaaaaaactggttgactctaccgacaaagctgacctgcgtctgatctacctggctctggctcacatgatcaaattccgtggtcacttcctgatcgaaggtgacctgaacccggacaactctgacgttgacaaactgttcatccagctggttcagacctacaaccagctgttcgaagaaaacccgatcaacgcttctggtgttgacgctaaagctatcctgtctgctcgtctgtctaaatctcgtcgtctggaaaacctgatcgctcagctgccgggtgaaaaaaaaaacggtctgttcggtaacctgatcgctctgtctctgggtctgaccccgaacttcaaatctaacttcgacctggctgaagacgctaaactgcaactgtctaaagacacctacgacgacgacctggacaacctgctggctcagatcggtgaccagtacgctgacctgttcctggctgctaaaaacctgtctgacgctatcctgctgtctgacatcctgcgtgttaacaccgaaatcaccaaagctccgctgtctgcttctatgatcaaacgttacgacgaacaccaccaggacctgaccctgctgaaagctctggttcgtcagcagctgccggaaaaatacaaagaaatcttcttcgaccagtctaaaaacggttacgctggttacatcgacggtggtgcttctcaggaagaattttacaaattcatcaaaccgatcctggaaaaaatggacggtactgaagaactgctggttaaactgaaccgtgaagacctgctgcgtaaacagcgtaccttcgacaacggttctatcccgcaccagatccacctgggtgaactgcacgctatcctgcgtcgtcaggaagacttctacccgttcctgaaagacaaccgtgaaaaaatcgaaaaaatcctgaccttccgtatcccgtactacgttggtccgctggctcgtggtaactctcgtttcgcttggatgacccgtaaatctgaagaaaccatcaccccgtggaacttcgaagaagttgttgacaaaggtgcttctgctcagtctttcatcgaacgtatgaccaacttcgacaaaaacctgccgaacgaaaaagttctgccgaaacactctctgctgtacgaatacttcaccgtttacaacgaactgaccaaagttaaatacgttaccgaaggtatgcgtaaaccggctttcctgtctggtgaacagaaaaaagctatcgttgacctgctgttcaaaaccaaccgtaaagttaccgttaaacagctgaaagaagactacttcaaaaaaatcgaatgcttcgactctgttgaaatctctggtgttgaagaccgtttcaacgcttctctgggtacttaccacgacctgctgaaaatcatcaaagacaaagacttcctggacaacgaagaaaacgaagacatcctggaagacatcgttctgaccctgaccctgttcgaagaccgtgaaatgatcgaagaacgtctgaaaacctacgctcacctgttcgacgacaaagttatgaaacagctgaaacgtcgtcgttacaccggttggggtcgtctgtctcgtaaactgatcaacggtatccgtgacaaacagtctggtaaaaccatcctggacttcctgaaatctgacggtttcgctaaccgtaacttcatgcagctgatccacgacgactctctgaccttcaaagaagacatccagaaggcgcaggtaagcggtcagggtgactctctgcacgaacacatcgctaacctggctggttctccggctatcaaaaaaggtatcctgcaaaccgttaaagttgttgacgaactggttaaagttatgggtcgtcacaaaccggaaaacatcgttatcgaaatggctcgtgaaaaccagaccacccagaaaggtcagaaaaactctcgtgaacgtatgaaacgtatcgaagaaggtatcaaagaactgggttctcagatcctgaaagaacacccggttgaaaacacccagctgcaaaacgaaaaactgtacctgtactacctgcaaaacggtcgtgacatgtacgttgaccaggaactggacatcaaccgtctgtctgactacgacgttgacgctatcgttccgcagtctttcctgaaagacgactctatcgacaacaaagttctgacccgttctgacaaaaaccgtggtaaatctgacaacgttccgtctgaagaagttgttaaaaaaatgaaaaactactggcgtcagctgctgaacgctaaactgatcacccagcgtaaattcgacaacctgaccaaagctgaacgtggtggtctgtctgaactggacaaagctggtttcatcaaacgtcagctggttgaaacccgtcagatcaccaaacacgttgctcagatcctggactctcgtatgaacaccaaatacgacgaaaacgacaaactgatccgtgaagttaaagttatcaccctgaaatctaaactggtttctgacttccgtaaagacttccagttctacaaagttcgtgaaatcaacaactaccaccacgctcacgacgcttacctgaacgctgttgttggtactgctctgatcaaaaaatacccgaaactggagtcggaatttgtgtacggggactacaaagtttacgacgttcgtaaaatgatcgctaaatctgaacaggaaatcggtaaagctaccgctaaatacttcttctactctaacatcatgaacttcttcaaaaccgaaatcaccctggctaacggtgaaatccgtaaacgtccgctgatcgaaaccaacggtgaaaccggcgagatcgtctgggacaaaggccgtgacttcgctaccgttcgtaaagttctgtctatgccgcaggttaacatcgttaaaaaaaccgaagttcagaccggtggtttctctaaagaatctatcctgccgaaacgtaactctgacaaactgatcgctcgtaaaaaagactgggacccaaaaaaatacggtggcttcgactctccgactgttgcttactctgttctggttgttgctaaagttgaaaaaggtaaatctaaaaaactgaaatctgttaaagaactgctgggtatcaccatcatggaacgttcttctttcgaaaaaaacccgatcgacttcctggaagctaaaggttacaaagaagttaaaaaagacctgatcatcaaactgccgaaatactctctgttcgaactggaaaacggtcgtaaacgtatgctggcttctgctggtgaactgcaaaaaggtaacgaactggctctgccgtctaaatacgttaacttcctgtacctggcttctcactacgaaaaactgaaaggttctccggaagacaacgaacagaaacagctgttcgttgaacagcacaaacactacctggacgaaatcatcgaacagatcagtgaattttctaaacgtgttatcctggctgacgctaacctggacaaagttctgtctgcttacaacaaacaccgtgacaaaccgatccgtgaacaggctgaaaacatcatccacctgttcaccctgaccaacctcggtgctccagctgcgttcaaatacttcgacaccaccatcgaccgtaaacgttacacctctaccaaagaagttctggacgctaccctgatccaccagtctatcaccggtctgtacgaaacccgtatcgacctgtctcagctgggtggtgactaaggatccgagctccaattcgccctatagtgagacgcgtgctagaggcatcaaataaaacgaaaggctcagtcgaaagactgggcctttcgttttatctgttgtttgtcggtgaacgcttaagtcgaacagaaagtaatcgtattgtacacggccgcataatcgaaattaggtaccctcgagtctggtaa

SEQ ID NO: 19 - DNA, 40 bp, synthetic construct
  misc_feature 1..40: note = The sequence is synthetized.
  source 1..40: mol_type = other DNA
ttaccagactcgagggtacctaatttcgattatgcggccg

SEQ ID NO: 20 - DNA, 20 bp, synthetic construct
  misc_feature 1..20: note = The sequence is synthetized.
  source 1..20: mol_type = other DNA
ggtaccctcgagtctggtaa

SEQ ID NO: 21 - DNA, 861 bp, synthetic construct
  misc_feature 1..861: note = The sequence is synthetized.
  source 1..861: mol_type = other DNA
atgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcacgcggtatcattgcagcactggggccagatggtaagccctcccgtatcgtagttatctacacgacggggagtcaggcaactatggatgaacgaaatagacagatcgctgagataggtgcctcactgattaagcattggtaa

SEQ ID NO: 22 - DNA, 2239 bp, synthetic construct
  misc_feature 1..2239: note = The sequence is synthetized.
  source 1..2239: mol_type = other DNA
ccactgatttccgctgctgcgttttagagctagaaatagcaagttaaaataaggctagtccgttatcaacttgaaaaagtggcaccgagtcggtgctttttttcaaataaaacgaaaggctcagtcgaaagactgggcctttcgttttatctgttgtttgtcggtgaactactagtagcggccgcctgcagcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaggtaccgcctaagcttgcttggattctcaccaataaaaaacgcccggcggcaaccgagcgttctgaacaaatccagatggagttctgaggtcattactggatctatcaacaggagtccatatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgtgacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctggagctcgcctgaattcgcggccgcttctagagtaacaccgtgcgtgttgactattttacctctggcggtgataatggttgcccactgatttccgctgctgc

SEQ ID NO: 23 - DNA, 43 bp, synthetic construct
  misc_feature 1..43: note = The sequence is synthetized.
  source 1..43: mol_type = other DNA
ccactgatttccgctgctgcgttttagagctagaaatagcaag

SEQ ID NO: 24 - DNA, 37 bp, synthetic construct
  misc_feature 1..37: note = The sequence is synthetized.
  source 1..37: mol_type = other DNA
gcagcagcggaaatcagtgggcaaccattatcaccgc

SEQ ID NO: 25 - DNA, 27 bp, synthetic construct
  misc_feature 1..27: note = The sequence is synthetized.
  source 1..27: mol_type = other DNA
gagatctgccatatgtatatctccttc

SEQ ID NO: 26 - DNA, 18 bp, synthetic construct
  misc_feature 1..18: note = The sequence is synthetized.
  source 1..18: mol_type = other DNA
aattggatatcggccggc

SEQ ID NO: 27 - DNA, 5129 bp, synthetic construct
  misc_feature 1..5129: note = The sequence is synthetized.
  source 1..5129: mol_type = other DNA
aattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggcagcagccatcaccatcatcaccacagccaggatccgatgaatcagccgctgaacgtggcgccgccggtgagcagcgaactgaacttacgcgcgcattggatgccgtttagcgcgaaccgcaactttcagaaagatccgcgcattattgtggcggcggaaggcagctggctgaccgatgataaaggccgcaaagtgtatgatagcctgagcggcctgtggacctgcggcgcgggccatagccgcaaagaaattcaagaagcggtggcgcgtcagctgggcaccctggattatagcccgggctttcagtatggccatccgctgagctttcagctggcggaaaaaattgcgggcctgctgccgggcgaactgaaccatgtgttttttaccggcagcggcagcgaatgcgcggatacgagcattaaaatggcgcgcgcgtattggcgcctgaaaggtcagccgcagaaaaccaaactgattggccgcgcgcgcggctatcatggcgtgaacgtggcgggcacgagcctgggcggcattggcggcaaccgcaaaatgtttggtcagctgatggatgtggatcatctgccgcataccctgcagccgggcatggcgtttacccgcggcatggcgcagaccggcggcgtggaactggcgaacgaactgctgaaactgattgaactgcatgatgcgagcaacattgcggcggtgattgtggaaccgatgagcggcagcgcgggcgtgctggtgccgccggtgggctatctgcagcgcctgcgcgaaatttgcgatcagcataacattctgctgatttttgatgaagtgattaccgcgtttggccgcctgggcacctatagcggcgcggaatattttggcgtgaccccggatctgatgaacgtggcgaaacaagtgaccaacggcgcggtgccgatgggcgcggtgattgcgagcagcgaaatttatgatacctttatgaaccaagcgctgccggaacatgcggtggaatttagccatggctatacctatagcgcgcatccggtggcgtgcgcggcgggcctggcggcgctggatattctggcgcgcgataacctggtgcagcagagcgcggaactggcgccgcattttgaaaaaggcctgcatggcctgcaaggcgcgaaaaacgtgattgatattcgcaactgcggcctggcgggcgcgattcagattgcgccgcgcgatggcgatccgaccgtgcgcccgtttgaagcgggcatgaaactgtggcagcaaggcttttatgtgcgctttggcggcgataccctgcagtttggcccgacctttaacgcgcgcccggaagaactggatcgcctgtttgatgcggtgggcgaagcgctgaacggcattgcgtaagaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctc

SEQ ID NO: 28 - DNA, 2051 bp, synthetic construct
  misc_feature 1..2051: note = The sequence is synthetized.
  source 1..2051: mol_type = other DNA
atatacatatggcagatctcagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcgtggtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgctggattgccctgagtgatggtgcccgtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaaaattggatatcggccggc

SEQ ID NO: 29 - DNA, 37 bp, synthetic construct
  misc_feature 1..37: note = The sequence is synthetized.
  source 1..37: mol_type = other DNA
atatacatatggcagatctcagcgccaccaccggcgc

SEQ ID NO: 30 - DNA, 38 bp, synthetic construct
  misc_feature 1..38: note = The sequence is synthetized.
  source 1..38: mol_type = other DNA
gccggccgatatccaattttacacggtaatcgcccgtc

SEQ ID NO: 31 - DNA, 816 bp, synthetic construct
  misc_feature 1..816: note = The sequence is synthetized.
  source 1..816: mol_type = other DNA
atgagccatattcaacgggaaacgtcttgctcccgtccgcgcttaaactccaacatggacgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtccgtctcaactggctgacggagtttatgcctctcccgaccatcaagcattttatccgtactcctgatgatgcgtggttactcaccaccgcgattcctgggaaaacagccttccaggtattagaagaatatcctgattcaggtgaaaatattgttgatgcgctggccgtgttcctgcgccggttacattcgattcctgtttgtaattgtccttttaacagcgatcgtgtatttcgtcttgctcaggcgcaatcacgcatgaataacggtttggttgatgcgagtgattttgatgacgagcgtaatggctggcctgttgaacaagtctggaaagaaatgcacaagctcttgccattctcaccggattcagtcgtcactcatggtgatttctcacttgataaccttatttttgacgaggggaaattaataggttgtattgatgttggacgggtcggaatcgcagaccgttaccaggaccttgccattctttggaactgcctcggtgagttttctccttcattacagaaacggctttttcaaaaatatggtattgataatcctgatatgaataaattgcagtttcatttgatgctcgatgagtttttctaa

SEQ ID NO: 32 - DNA, 4461 bp, synthetic construct
  misc_feature 1..4461: note = The sequence is synthetized.
  source 1..4461: mol_type = other DNA
tgatgataagccaggcatcaaataaaacgaaaggctcagtcgaaagactgggcctttcgttttatctgttgtttgtcggtgaacgctctctactagagtcacactggctcaccttcgggtgggcctttctgcgtttatactgcaggagtcactaagggttagttagttagattagcagaaagtcaaaagcctccgaccggaggcttttgactaaaacttcccttggggttatcattggggctcactcaaaggcggtaatcagataaaaaaaatccttagctttcgctaaggatgatttctgctagtattattagaaaaactcatcgagcatcaaatgaaactgcaatttattcatatcaggattatcaataccatatttttgaaaaagccgtttctgtaatgaaggagaaaactcaccgaggcagttccaaagaatggcaaggtcctggtaacggtctgcgattccgacccgtccaacatcaatacaacctattaatttcccctcgtcaaaaataaggttatcaagtgagaaatcaccatgagtgacgactgaatccggtgagaatggcaagagcttgtgcatttctttccagacttgttcaacaggccagccattacgctcgtcatcaaaatcactcgcatcaaccaaaccgttattcatgcgtgattgcgcctgagcaagacgaaatacacgatcgctgttaaaaggacaattacaaacaggaatcgaatgtaaccggcgcaggaacacggccagcgcatcaacaatattttcacctgaatcaggatattcttctaatacctggaaggctgttttcccaggaatcgcggtggtgagtaaccacgcatcatcaggagtacggataaaatgcttgatggtcgggagaggcataaactccgtcagccagttgagacggaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcgtccatgttggagtttaagcgcggacgggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagctctagtagttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattacatgtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtccctagtgcttggattctcaccaataaaaaacgcccggcggcaaccgagcgttctgaacaaatccagatggagttctgaggtcattactggatctatcaacaggagtccaagcgagctcgtaaacttggtctgacagctctagctccggcaaaaaaacgggcaaggtgtcaccaccctgccctttttctttaaaaccgaaaagattacttcgcgtttgccacctgacgtctaagaaaaggaatattcagcaatttgcccgtgccgaagaaaggcccacccgtgaaggtgagccagtgagttgattgctacgtaattagttagttagcccttagtgactcgaattctttacggctagctcagtcctaggtatagtgctagctactagagaaagaggagaaatactagatggtaataatgagcccaaaaagaagaacacaagcagaaagagcaatggaaacacaaggaaaactaatagcagcagcactaggagtactaagagaaaaaggatacgcaggattcagaatagcagacgtaccaggagcagcaggagtaagcagaggagcacaaagccaccacttcccaacaaaactagaactactactagcaacattcgaatggctatacgaacaaataacagaaagaagcagagcaagactagcaaaactaaaaccagaggacgacgtaatacaacaaatgctagacgacgcagcagagttcttcctagacgacgacttcagcataagcctagacctaatagtagcagcagaccgcgacccagcactaagagaaggaatacaaagaacagtagaaagaaacagattcgtagtagaggacatgtggctaggagtgctagtaagcagaggactaagcagagacgacgcagaggacatactatggctaatattcaacagcgtaagaggactagcagtaagaagcctatggcaaaaagacaaagaaagattcgaaagagtaagaaacagcacactagaaatagcaagagaaagatacgcaaaattcaaaagataataactcggtaccaaattccagaaaagaggcctcccgaaaggggggccttttttcgttttggtcctactagaggttatgagtcaggaaaaaaggcgacagagtaatctgtcgccttttttctttgcttgcttttactagagtcataaaaaatttatttgctttgtggcggataacaattataatagattcaacaaacagacaatctggtctgtttgtatttactagagatttaaattcttttaaggagtaagt

SEQ ID NO: 33 - DNA, 36 bp, synthetic construct
  misc_feature 1..36: note = The sequence is synthetized.
  source 1..36: mol_type = other DNA
acttactccttaaaagaatttaaatctctagtaaat

SEQ ID NO: 34 - DNA, 21 bp, synthetic construct
  misc_feature 1..21: note = The sequence is synthetized.
  source 1..21: mol_type = other DNA
tgatgataagccaggcatcaa

SEQ ID NO: 35 - DNA, 1092 bp, synthetic construct
  misc_feature 1..1092: note = The sequence is synthetized.
  source 1..1092: mol_type = other DNA
aaattcttttaaggagtaagtatgagcaccctgtgcctgccgcatgtgatgtttccgcagcataaaattacgcagcaacagaccgtggatcatctggaaaacctgcatgcggatcatccgcgcatggcgctggcgaaacgcatgattgcgaacaccgaagtgaacgaacgccatctggtggtgccgattgatgaactggcggtgcataccggctttacccatcgcagcattgtgtatgaacgcgaagcgcgtcagatgagcagcaccgcggcgcgccaagcgattgaaaacgcgggcctgcagattagcgatattcgcatggtgattgtgacgagctgcaccggctttatgatgccgagcctgaccgcgcatctgattaacgatctggccctgccgacgagcaccgtgcagctgccgattgcgcagttaggctgcgtggcgggcgcggccgcgattaaccgcgcgaacgattttgcgcgcctggatgcgcgcaaccatgtgctgattgtgagcctggaatttagcagcctgtgctatcagccggatgataccaaactgcatagctttattagcgcggcgctgtttggcgatgcggtgagcgcgtgcgtgctgcgcgcggatgatcaagcgggcggctttaaaattaaaaaaaccgaaagctattttctgccgaaaagcgaacattatattaaatatgatgtgaaagataccggctttcattttaccctggataaagcggtgatgaacagcattaaagatgtggcgccggtgatggaacgcctgaactatgaaagctttgaacagaactgcgcgcataacgatttttttatttttcataccggcggccgcaaaattctggatgaactggtgatgcatctggatctggcgagcaaccgcgtgagtcagagccgcagcagcctgagcgaagcgggcaacattgcgagcgtggttgtgtttgatgtgctgaaacgtcagtttgatagcaacctgaaccgcggcgatattggcctgctggcggcgtttggcccgggctttaccgcggaaatggcggtgggcgaatggaccgcgtaatgatgataagccaggcatcaa

SEQ ID NO: 36 - DNA, 39 bp, synthetic construct
  misc_feature 1..39: note = The sequence is synthetized.
  source 1..39: mol_type = other DNA
aaattcttttaaggagtaagtatgagcaccctgtgcctg

SEQ ID NO: 37 - DNA, 39 bp, synthetic construct
  misc_feature 1..39: note = The sequence is synthetized.
  source 1..39: mol_type = other DNA
ttgatgcctggcttatcatcattacgcggtccattcgcc

SEQ ID NO: 38 - DNA, 1169 bp, synthetic construct
  misc_feature 1..1169: note = The sequence is synthetized.
  source 1..1169: mol_type = other DNA
agagatttaaattcttttaaggagtaagtatggcgaccctgtgccgtccggcaatcgcggttccggaacacgttatcaccatgcagcagaccctggatctggctcgtgaaacccacgcgggccacccgcagcgtgatctggttctgcgtctgatccagaacaccggcgttcagacccgtcacctggtgcagccgatcgaaaaaaccctggcgcatccgggtttcgaagttcgtaaccaggtgtatgaagcggaagcgaaaacccgtgttccggaagttgttcgtcgtgcactggcgaacgcggaaaccgaaccgagcgaaatcgacctgatcgtttacgttagctgcaccggtttcatgatgccatctctgaccgcgtggatcatcaactccatgggtttccgtccggaaacccgtcagctgccgatcgcgcagctgggctgcgcggctggcggcgcggcgatcaaccgtgcgcacgacttctgcgttgcgtacccggatagcaacgttctgatcgtttcctgcgaattttgctctctgtgctaccagccgaccgacattggcgttggcagcctgctgtctaacggtctgttcggcgacgcgctgagcgcggcggttgtgcgtggtcagggcggcaccggcatgcgtctggaacgcaacggttctcacctggtgccggataccgaagattggatcagctacgcggtgcgtgataccggtttccatttccagctggataaacgtgttccgggcaccatggaaatgctggcgccggtgctgctggatctggttgatctgcacggttggtctgttccgaacatggatttcttcattgttcacgcgggcggtccgcgtatcctggatgatctgtgccacttcctggatctgccgccggaaatgttccgctacagccgtgctaccctgaccgaacgcggcaacatcgcttcttccgttgttttcgatgcgctggcgcgcctgttcgatgatggtggcgcggcggaatctgcgcagggtctgatcgcgggcttcggtccgggtatcaccgccgaagtggcggttggctcttgggcgaaagaaggcctgggcgcggatgttggtcgtgatctggatgaactcgaactgaccgcgggtgttgcgctgagcggttaatgatgataagccaggcatcaa

SEQ ID NO: 39 - DNA, 47 bp, synthetic construct
  misc_feature 1..47: note = The sequence is synthetized.
  source 1..47: mol_type = other DNA
agagatttaaattcttttaaggagtaagtatggcgaccctgtgccgt

SEQ ID NO: 40 - DNA, 44 bp, synthetic construct
  misc_feature 1..44: note = The sequence is synthetized.
  source 1..44: mol_type = other DNA
ttgatgcctggcttatcatcattaaccgctcagcgcaacacccg

SEQ ID NO: 41 - DNA, 5709 bp, synthetic construct
  misc_feature 1..5709: note = The sequence is synthetized.
  source 1..5709: mol_type = other DNA
gatccgcggccgcgcgcgatatcgaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggttcatgtgcagctccatcagcaaaaggggatgataagtttatcaccaccgactatttgcaacagtgccgttgatcgtgctatgatcgactgatgtcatcagcggtggagtgcaatgtcgtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagaggcgggatgcgaagaatgcgatgccgctcgccagtcgattggctgagctcatgagcggagaacgagatgacgttggaggggcaaggtcgcgctgattgctggggcaacacgtggagcggatcggggattgtctttcttcagctcgctgatgatatgctgacgctcaatgccgtttggcctccgactaacgaaaatcccgcatttggacggctgatccgattggcacggcggacggcgaatggcggagcagacgctcgtccgggggcaatgagatatgaaaaagcctgaactcaccgcgacgtatcgggccctggccagctagctagagtcgacctgcaggtccccggggatcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgacctgcaggcatgcaagctctagcgattccagacgtcccgaaggcgtggcgcggcttccccgtgccggagcaatcgccctgggtgggttacacgacgcccctctatggcccgtactgacggacacaccgaagccccggcggcaaccctcagcggatgccccggggcttcacgttttcccaggtcagaagcggttttcgggagtagtgccccaactggggtaacctttgagttctctcagttgggggcgtagggtcgccgacatgacacaaggggttgtgaccggggtggacacgtacgcgggtgcttacgaccgtcagtcgcgcgagcgcgagaattcgagcgcagcaagcccagcgacacagcgtagcgccaacgaagacaaggcggccgaccttcagcgcgaagtcgagcgcgacgggggccggttcaggttcgtcgggcatttcagcgaagcgccgggcacgtcggcgttcgggacggcggagcgcccggagttcgaacgcatcctgaacgaatgccgcgccgggcggctcaacatgatcattgtctatgacgtgtcgcgcttctcgcgcctgaaggtcatggacgcgattccgattgtctcggaattgctcgccctgggcgtgacgattgtttccactcaggaaggcgtcttccggcagggaaacgtcatggacctgattcacctgattatgcggctcgacgcgtcgcacaaagaatcttcgctgaagtcggcgaagattctcgacacgaagaaccttcagcgcgaattgggcgggtacgtcggcgggaaggcgccttacggcttcgagcttgtttcggagacgaaggagatcacgcgcaacggccgaatggtcaatgtcgtcatcaacaagcttgcgcactcgaccactccccttaccggacccttcgagttcgagcccgacgtaatccggtggtggtggcgtgagatcaagacgcacaaacaccttcccttcaagccgggcagtcaagccgccattcacccgggcagcatcacggggctttgtaagcgcatggacgctgacgccgtgccgacccggggcgagacgattgggaagaagaccgcttcaagcgcctgggacccggcaaccgttatgcgaatccttcgggacccgcgtattgcgggcttcgccgctgaggtgatctacaagaagaagccggacggcacgccgaccacgaagattgagggttaccgcattcagcgcgacccgatcacgctccggccggtcgagcttgattgcggaccgatcatcgagcccgctgagtggtatgagcttcaggcgtggttggacggcagggggcgcggcaaggggctttcccgggggcaagccattctgtccgccatggacaagctgtactgcgagtgtggcgccgtcatgacttcgaagcgcggggaagaatcgatcaaggactcttaccgctgccgtcgccggaaggtggtcgacccgtccgcacctgggcagcacgaaggcacgtgcaacgtcagcatggcggcactcgacaagttcgttgcggaacgcatcttcaacaagatcaggcacgccgaaggcgacgaagagacgttggcgcttctgtgggaagccgcccgacgcttcggcaagctcactgaggcgcctgagaagagcggcgaacgggcgaaccttgttgcggagcgcgccgacgccctgaacgcccttgaagagctgtacgaagaccgcgcggcaggcgcgtacgacggacccgttggcaggaagcacttccggaagcaacaggcagcgctgacgctccggcagcaaggggcggaagagcggcttgccgaacttgaagccgccgaagccccgaagcttccccttgaccaatggttccccgaagacgccgacgctgacccgaccggccctaagtcgtggtgggggcgcgcgtcagtagacgacaagcgcgtgttcgtcgggctcttcgtagacaagatcgttgtcacgaagtcgactacgggcagggggcagggaacgcccatcgagaagcgcgcttcgatcacgtgggcgaagccgccgaccgacgacgacgaagacgacgcccaggacggcacggaagacgtagcggcgtagcgagacacccgggaagcctgatctacgtctgtcgagaagtttctgatcgaaaagttcgacagcgtctccgacctgatgcagctctcgcagggcgaagaatctcgtgctttcagcttcgatgtaggagggcgtggatatgtcctgcgggtaaatagctgcgccgatggtttctacaaagatcgttatgttgatcggcactttgcatcggccgcgctcccgattccggaagtgcttgacattggggaatttatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgggctgcaggtcgact

SEQ ID NO: 42 - DNA, 149 bp, synthetic construct
  misc_feature 1..149: note = The sequence is synthetized.
  source 1..149: mol_type = other DNA
gggctgcaggtcgactctagaggaacgatcgttggctgtgttcacattcgaaccgtctctgctttgacaacatgctgtgcggtgttgtaaagtcgtggccaggagaatacgacaggtatctgaaaggggatacgccatatgaatcagcc

SEQ ID NO: 43 - DNA, 127 bp, synthetic construct
  misc_feature 1..127: note = The sequence is synthetized.
  source 1..127: mol_type = other DNA
cattgcgtaatatctgttcacattcgaaccgtctctgctttgacaacatgctgtgcggtgttgtaaagtcgggtgaaggagaatacgacagtctaagtaaggagtgtccatcatatgatggcagatc

SEQ ID NO: 44 - DNA, 1368 bp, synthetic construct
  misc_feature 1..1368: note = The sequence is synthetized.
  source 1..1368: mol_type = other DNA
gatacgccatatgaatcagccgctgaacgtggcgccgccggtgagcagcgaactgaacttacgcgcgcattggatgccgtttagcgcgaaccgcaactttcagaaagatccgcgcattattgtggcggcggaaggcagctggctgaccgatgataaaggccgcaaagtgtatgatagcctgagcggcctgtggacctgcggcgcgggccatagccgcaaagaaattcaagaagcggtggcgcgtcagctgggcaccctggattatagcccgggctttcagtatggccatccgctgagctttcagctggcggaaaaaattgcgggcctgctgccgggcgaactgaaccatgtgttttttaccggcagcggcagcgaatgcgcggatacgagcattaaaatggcgcgcgcgtattggcgcctgaaaggtcagccgcagaaaaccaaactgattggccgcgcgcgcggctatcatggcgtgaacgtggcgggcacgagcctgggcggcattggcggcaaccgcaaaatgtttggtcagctgatggatgtggatcatctgccgcataccctgcagccgggcatggcgtttacccgcggcatggcgcagaccggcggcgtggaactggcgaacgaactgctgaaactgattgaactgcatgatgcgagcaacattgcggcggtgattgtggaaccgatgagcggcagcgcgggcgtgctggtgccgccggtgggctatctgcagcgcctgcgcgaaatttgcgatcagcataacattctgctgatttttgatgaagtgattaccgcgtttggccgcctgggcacctatagcggcgcggaatattttggcgtgaccccggatctgatgaacgtggcgaaacaagtgaccaacggcgcggtgccgatgggcgcggtgattgcgagcagcgaaatttatgatacctttatgaaccaagcgctgccggaacatgcggtggaatttagccatggctatacctatagcgcgcatccggtggcgtgcgcggcgggcctggcggcgctggatattctggcgcgcgataacctggtgcagcagagcgcggaactggcgccgcattttgaaaaaggcctgcatggcctgcaaggcgcgaaaaacgtgattgatattcgcaactgcggcctggcgggcgcgattcagattgcgccgcgcgatggcgatccgaccgtgcgcccgtttgaagcgggcatgaaactgtggcagcaaggcttttatgtgcgctttggcggcgataccctgcagtttggcccgacctttaacgcgcgcccggaagaactggatcgcctgtttgatgcggtgggcgaagcgctgaacggcattgcgtaatatctgttcac

SEQ ID NO: 45 - DNA, 2057 bp, synthetic construct
  misc_feature 1..2057: note = The sequence is synthetized.
  source 1..2057: mol_type = other DNA
ccatcatatgatggcagatctcagcgccaccaccggcgcacgcagtgcatcggtcggatgggcggaaagcctgatcgggttgcatttggggaaagttgccttgattaccggtggcagcgccggtattggtgggcagatcgggcgcctcctggctttgagtggcgcgcgcgtgatgctggcagcccgtgatcggcataagctcgaacagatgcaggcgatgatccaatctgagctggctgaggtggggtataccgatgtcgaagatcgcgtccacattgcaccgggctgcgatgtgagtagcgaagcgcagcttgcggatcttgttgaacgtaccctgtcagcttttggcaccgtcgattatctgatcaacaacgccgggatcgccggtgtcgaagagatggttatcgatatgccagttgagggatggcgccataccctcttcgccaatctgatcagcaactactcgttgatgcgcaaactggcgccgttgatgaaaaaacagggtagcggttacatccttaacgtctcatcatactttggcggtgaaaaagatgcggccattccctaccccaaccgtgccgattacgccgtctcgaaggctggtcagcgggcaatggccgaagtctttgcgcgcttccttggcccggagatacagatcaatgccattgcgccgggtccggtcgaaggtgatcgcttgcgcggtaccggtgaacgtcccggcctctttgcccgtcgggcgcggctgattttggagaacaagcggctgaatgagcttcacgctgctcttatcgcggctgcgcgcaccgatgagcgatctatgcacgaactggttgaactgctcttacccaatgatgtggccgcactagagcagaatcccgcagcacctaccgcgttgcgtgaactggcacgacgttttcgcagcgaaggcgatccggcggcatcatcaagcagtgcgctgctgaaccgttcaattgccgctaaattgctggctcgtttgcataatggtggctatgtgttgcctgccgacatctttgcaaacctgccaaacccgcccgatcccttcttcacccgagcccagattgatcgcgaggctcgcaaggttcgtgacggcatcatggggatgctctacctgcaacggatgccgactgagtttgatgtcgcaatggccaccgtctattaccttgccgaccgcgtggtcagtggtgagacattccacccatcaggtggtttgcgttacgaacgcacccctaccggtggcgaactcttcggcttgccctcaccggaacggctggcggagctggtcggaagcacggtctatctgataggtgaacatctgactgaacaccttaacctgcttgcccgtgcgtacctcgaacgttacggggcacgtcaggtagtgatgattgttgagacagaaaccggggcagagacaatgcgtcgcttgctccacgatcacgtcgaggctggtcggctgatgactattgtggccggtgatcagatcgaagccgctatcgaccaggctatcactcgctacggtcgcccagggccggtcgtctgtacccccttccggccactgccgacggtaccactggtcgggcgtaaagacagtgactggagcacagtgttgagtgaggctgaatttgccgagttgtgcgaacaccagctcacccaccatttccgggtagcgcgctggattgccctgagtgatggtgcccgtctcgcgctggtcactcccgaaactacggctacctcaactaccgagcaatttgctctggctaacttcatcaaaacgacccttcacgcttttacggctacgattggtgtcgagagcgaaagaactgctcagcgcattctgatcaatcaagtcgatctgacccggcgtgcgcgtgccgaagagccgcgtgatccgcacgagcgtcaacaagaactggaacgttttatcgaggcagtcttgctggtcactgcaccactcccgcctgaagccgatacccgttacgccgggcggattcatcgcggacgggcgattaccgtgtaaggatccgcggccgcgcgcgata

SEQ ID NO: 46 - DNA, 40 bp, synthetic construct
  misc_feature 1..40: note = The sequence is synthetized.
  source 1..40: mol_type = other DNA
gggctgcaggtcgactctagaggaacgatcgttggctgtg

SEQ ID NO: 47 - DNA, 30 bp, synthetic construct
  misc_feature 1..30: note = The sequence is synthetized.
  source 1..30: mol_type = other DNA
ggctgattcatatggcgtatcccctttcag

SEQ ID NO: 48 - DNA, 29 bp, synthetic construct
  misc_feature 1..29: note = The sequence is synthetized.
  source 1..29: mol_type = other DNA
cattgcgtaatatctgttcacattcgaac

SEQ ID NO: 49 - DNA, 31 bp, synthetic construct
  misc_feature 1..31: note = The sequence is synthetized.
  source 1..31: mol_type = other DNA
gatctgccatcatatgatggacactccttac

SEQ ID NO: 50 - DNA, 28 bp, synthetic construct
  misc_feature 1..28: note = The sequence is synthetized.
  source 1..28: mol_type = other DNA
gatacgccatatgaatcagccgctgaac

SEQ ID NO: 51 - DNA, 29 bp, synthetic construct
  misc_feature 1..29: note = The sequence is synthetized.
  source 1..29: mol_type = other DNA
gtgaacagatattacgcaatgccgttcag

SEQ ID NO: 52 - DNA, 30 bp, synthetic construct
  misc_feature 1..30: note = The sequence is synthetized.
  source 1..30: mol_type = other DNA
ccatcatatgatggcagatctcagcgccac

SEQ ID NO: 53 - DNA, 42 bp, synthetic construct
  misc_feature 1..42: note = The sequence is synthetized.
  source 1..42: mol_type = other DNA
tatcgcgcgcggccgcggatccttacacggtaatcgcccgtc

SEQ ID NO: 54 - DNA, 19 bp, synthetic construct
  misc_feature 1..19: note = The sequence is synthetized.
  source 1..19: mol_type = other DNA
ggatctcgacgctctccct

SEQ ID NO: 55 - DNA, 20 bp, synthetic construct
  misc_feature 1..20: note = The sequence is synthetized.
  source 1..20: mol_type = other DNA
cagggtacgttcaacaagat

SEQ ID NO: 56 - DNA, 20 bp, synthetic construct
  misc_feature 1..20: note = The sequence is synthetized.
  source 1..20: mol_type = other DNA
ttgtacacggccgcataatc

SEQ ID NO: 57 - DNA, 21 bp, synthetic construct
  misc_feature 1..21: note = The sequence is synthetized.
  source 1..21: mol_type = other DNA
aagcagaggactaagcagaga

SEQ ID NO: 58 - DNA, 21 bp, synthetic construct
  misc_feature 1..21: note = The sequence is synthetized.
  source 1..21: mol_type = other DNA
ctgattaccgcctttgagtga

SEQ ID NO: 59 - DNA, 19 bp, synthetic construct
  misc_feature 1..19: note = The sequence is synthetized.
  source 1..19: mol_type = other DNA
gacgacgagccgttcgatc

SEQ ID NO: 60 - DNA, 19 bp, synthetic construct
  misc_feature 1..19: note = The sequence is synthetized.
  source 1..19: mol_type = other DNA
cttctccttgagccacctg

SEQ ID NO: 61 - DNA, 1811 bp, synthetic construct
  misc_feature 1..1811: note = The sequence is synthetized.
  source 1..1811: mol_type = other DNA
caccacagccaggatccggtgtcagtcgagactaggaagatcaccaaggttcttgtcgctaaccgtggtgagattgcaatccgcgtgttccgtgcagctcgagatgaaggcatcggatctgtcgccgtctacgcagagccagatgcagatgcaccattcgtgtcatatgcagacgaggcttttgccctcggtggccaaacatccgctgagtcctaccttgtcattgacaagatcatcgatgcggcccgcaagtccggcgccgacgccatccaccccggctacggcttcctcgcagaaaacgctgacttcgcagaagcagtcatcaacgaaggcctgatctggattggaccttcacctgagtccatccgctccctcggcgacaaggtcaccgctcgccacatcgcagataccgccaaggctccaatggctcctggcaccaaggaaccagtaaaagacgcagcagaagttgtggctttcgctgaagaattcggtctcccaatcgccatcaaggcagctttcggtggcggcggacgtggcatgaaggttgcctacaagatggaagaagtcgctgacctcttcgagtccgcaacccgtgaagcaaccgcagcgttcggccgcggcgagtgcttcgtggagcgctacctggacaaggcacgccacgttgaggctcaggtcatcgccgataagcacggcaacgttgttgtcgccggaacccgtgactgctccctgcagcgccgtttccagaagctcgtcgaagaagcaccagcaccattcctcaccgatgaccagcgcgagcgtctccactcctccgcgaaggctatctgtaaggaagctggctactacggtgcaggcaccgttgagtacctcgttggctccgacggcctgatctccttcctcgaggtcaacacccgcctccaggtggaacacccagtcaccgaagagaccaccggcatcgacctggtccgcgaaatgttccgcatcgcagaaggccacgagctctccatcaaggaagatccagctccacgcggccacgcattcgagttccgcatcaacggcgaagacgctggctccaacttcatgcctgcaccaggcaagatcaccagctaccgcgagccacagggcccaggcgtccgcatggactccggtgtcgttgaaggttccgaaatctccggacagttcgactccatgctggcaaagctgatcgtttggggcgacacccgcgagcaggctctccagcgctcccgccgtgcacttgcagagtacgttgtcgagggcatgccaaccgttatcccattccaccagcacatcgtggaaaacccagcattcgtgggcaacgacgaaggcttcgagatctacaccaagtggatcgaagaggtttgggataacccaatcgcaccttacgttgacgcttccgagctcgacgaagatgaggacaagaccccagcacagaaggttgttgtggagatcaacggccgtcgcgttgaggttgcactcccaggcgatctggcactcggtggcaccgctggtcctaagaagaaggccaagaagcgtcgcgcaggtggtgcaaaggctggcgtatccggcgatgcagtggcagctccaatgcagggcactgtcatcaaggtcaacgtcgaagaaggcgctgaagtcaacgaaggcgacaccgttgttgtcctcgaggctatgaagatggaaaaccctgtgaaggctcataagtccggaaccgtaaccggccttactgtcgctgcaggcgagggtgtcaacaagggcgttgttctcctcgagatcaagtaaaattcgagctcggcgcg

SEQ ID NO: 62 - DNA, 40 bp, synthetic construct
  misc_feature 1..40: note = The sequence is synthetized.
  source 1..40: mol_type = other DNA
caccacagccaggatccggtgtcagtcgagactaggaaga

SEQ ID NO: 63 - DNA, 36 bp, synthetic construct
  misc_feature 1..36: note = The sequence is synthetized.
  source 1..36: mol_type = other DNA
cgcgccgagctcgaattttacagtggcatgttgccg

SEQ ID NO: 64 - DNA, 31 bp, synthetic construct
  misc_feature 1..31: note = The sequence is synthetized.
  source 1..31: mol_type = other DNA
atgtatatctccttcttatacttaactaata

SEQ ID NO: 65 - DNA, 20 bp, synthetic construct
  misc_feature 1..20: note = The sequence is synthetized.
  source 1..20: mol_type = other DNA
atggcagatctcaattggat

SEQ ID NO: 66 - DNA, 5557 bp, synthetic construct
  misc_feature 1..5557: note = The sequence is synthetized.
  source 1..5557: mol_type = other DNA
atggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactataggggaattgtgagcggataacaattcccctgtagaaataattttgtttaactttaataaggagatataccatgggcagcagccatcaccatcatcaccacagccaggatccggtgtcagtcgagactaggaagatcaccaaggttcttgtcgctaaccgtggtgagattgcaatccgcgtgttccgtgcagctcgagatgaaggcatcggatctgtcgccgtctacgcagagccagatgcagatgcaccattcgtgtcatatgcagacgaggcttttgccctcggtggccaaacatccgctgagtcctaccttgtcattgacaagatcatcgatgcggcccgcaagtccggcgccgacgccatccaccccggctacggcttcctcgcagaaaacgctgacttcgcagaagcagtcatcaacgaaggcctgatctggattggaccttcacctgagtccatccgctccctcggcgacaaggtcaccgctcgccacatcgcagataccgccaaggctccaatggctcctggcaccaaggaaccagtaaaagacgcagcagaagttgtggctttcgctgaagaattcggtctcccaatcgccatcaaggcagctttcggtggcggcggacgtggcatgaaggttgcctacaagatggaagaagtcgctgacctcttcgagtccgcaacccgtgaagcaaccgcagcgttcggccgcggcgagtgcttcgtggagcgctacctggacaaggcacgccacgttgaggctcaggtcatcgccgataagcacggcaacgttgttgtcgccggaacccgtgactgctccctgcagcgccgtttccagaagctcgtcgaagaagcaccagcaccattcctcaccgatgaccagcgcgagcgtctccactcctccgcgaaggctatctgtaaggaagctggctactacggtgcaggcaccgttgagtacctcgttggctccgacggcctgatctccttcctcgaggtcaacacccgcctccaggtggaacacccagtcaccgaagagaccaccggcatcgacctggtccgcgaaatgttccgcatcgcagaaggccacgagctctccatcaaggaagatccagctccacgcggccacgcattcgagttccgcatcaacggcgaagacgctggctccaacttcatgcctgcaccaggcaagatcaccagctaccgcgagccacagggcccaggcgtccgcatggactccggtgtcgttgaaggttccgaaatctccggacagttcgactccatgctggcaaagctgatcgtttggggcgacacccgcgagcaggctctccagcgctcccgccgtgcacttgcagagtacgttgtcgagggcatgccaaccgttatcccattccaccagcacatcgtggaaaacccagcattcgtgggcaacgacgaaggcttcgagatctacaccaagtggatcgaagaggtttgggataacccaatcgcaccttacgttgacgcttccgagctcgacgaagatgaggacaagaccccagcacagaaggttgttgtggagatcaacggccgtcgcgttgaggttgcactcccaggcgatctggcactcggtggcaccgctggtcctaagaagaaggccaagaagcgtcgcgcaggtggtgcaaaggctggcgtatccggcgatgcagtggcagctccaatgcagggcactgtcatcaaggtcaacgtcgaagaaggcgctgaagtcaacgaaggcgacaccgttgttgtcctcgaggctatgaagatggaaaaccctgtgaaggctcataagtccggaaccgtaaccggccttactgtcgctgcaggcgagggtgtcaacaagggcgttgttctcctcgagatcaagtaaaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacat

SEQ ID NO: 67 - DNA, 1683 bp, synthetic construct
  misc_feature 1..1683: note = The sequence is synthetized.
  source 1..1683: mol_type = other DNA
tattagttaagtataagaaggagatatacatatgaccatttcctcacctttgattgacgtcgccaaccttccagacatcaacaccactgccggcaagatcgccgaccttaaggctcgccgcgcggaagcccatttccccatgggtgaaaaggcagtagagaaggtccacgctgctggacgcctcactgcccgtgagcgcttggattacttactcgatgagggctccttcatcgagaccgatcagctggctcgccaccgcaccaccgctttcggcctgggcgctaagcgtcctgcaaccgacggcatcgtgaccggctggggcaccattgatggacgcgaagtctgcatcttctcgcaggacggcaccgtattcggtggcgcgcttggtgaggtgtacggcgaaaagatgatcaagatcatggagctggcaatcgacaccggccgcccattgatcggtctttacgaaggcgctggcgctcgtattcaggacggcgctgtctccctggacttcatttcccagaccttctaccaaaacattcaggcttctggcgttatcccacagatctccgtcatcatgggcgcatgtgcaggtggcaacgcttacggcccagctctgaccgacttcgtggtcatggtggacaagacctccaagatgttcgttaccggcccagacgtgatcaagaccgtcaccggcgaggaaatcacccaggaagagcttggcggagcaaccacccacatggtgaccgctggtaactcccactacaccgctgcgaccgatgaggaagcactggattgggtacaggacctggtgtccttcctcccatccaacaatcgctcctacgcaccgatggaagacttcgacgaggaagaaggcggcgttgaagaaaacatcaccgctgacgatctgaagctcgacgagatcatcccagattccgcgaccgttccttacgacgtccgcgatgtcatcgaatgcctcaccgacgatggcgaatacctggaaatccaggcagaccgcgcagaaaacgttgttattgcattcggccgcatcgaaggccagtccgttggctttgttgccaaccagccaacccagttcgctggctgcctggacatcgactcctctgagaaggcagctcgcttcgtccgcacctgcgacgcgttcaacatcccaatcgtcatgcttgtcgacgtccccggcttcctcccaggcgcaggccaggagtacggtggcattctgcgtcgtggcgcaaagctgctctacgcatacggcgaagcaaccgttccaaagatcaccgtcaccatgcgtaaggcttacggcggagcgtactgcgtgatgggttccaagggcttgggctctgacatcaaccttgcatggccaaccgcacagatcgccgtcatgggcgctgctggcgcagttggattcatctaccgcaaggagctcatggcagctgatgccaagggcctcgataccgtagctctggctaagtccttcgagcgcgagtatgaagaccacatgctcaacccgtaccacgctgcagaacgtggcctgatcgacgccgtgatcctgccaagcgaaacccgcggacagatttcccgcaaccttcgcctgctcaagcacaagaacgtcactcgccctgctcgcaagcacggcaacatgccactgtaaatggcagatctcaattggat

SEQ ID NO: 68 - DNA, 51 bp, synthetic construct
  misc_feature 1..51: note = The sequence is synthetized.
  source 1..51: mol_type = other DNA
tattagttaagtataagaaggagatatacatatgaccatttcctcaccttt

SEQ ID NO: 69 - DNA, 39 bp, synthetic construct
  misc_feature 1..39: note = The sequence is synthetized.
  source 1..39: mol_type = other DNA
atccaattgagatctgccatttacagtggcatgttgccg

SEQ ID NO: 70 - protein, 670 aa, synthetic construct
  REGION 1..670: note = The sequence is synthetized.
SATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRVVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARWIALSDGARLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 71 - protein, 452 aa, synthetic construct
  REGION 1..452: note = The sequence is synthetized.
MSDYLSIKEQDKQYVLHSWAKQGSFNPKVIAKAEGVCFWDDAGNRYFDMSSQLVYVNVGHQHPKVLKAFQEIGTYPLAGPSFATKPKSDLSQRIIELAPSNMAKVFYTSGGAEANDHAVKIARMVSGRYKIFSRYRSYHGATFGAGNLTGESRRFAAEPGLPGFVKFEVPYLYREVIDFDSEEAATAYYLGKLRNQILYEGSDTIAAIFIEPIPGSNGVLIPPKGYLEGVRALCDEFGILMICDEVMTGFGRTGKGFAVDHYDFKPDIMTFAKGVTSGYAPLGGVIISEDISRFFDENVLMTGLTYSGHTMCTQIGVANLEVYEEENIFENAEKMGILLKQRLEKLLTFPSIGEIRSIGLFAGVELVKDKATKEPIIQYGLDYGKDPVGLMNKFISMLSANGFYTYSHESTVIIAPPLIITEEQINEAMDIFESTLKQFEKEGLFPEETLSV

SEQ ID NO: 72 - protein, 475 aa, synthetic construct
  REGION 1..475: note = The sequence is synthetized.
MPSYSVAELYYPDEPTEPKISTSSYPGPKAKQELEKLSNVFDTRAAYLLADYYKSRGNYIVDQDGNVLLDVYAQISSIALGYNNPEILKVAKSDAMSVALANRPALACFPSNDYGQLLEDGLLKAAPQGQDKIWTALSGSDANETAFKACFMYQAAKKRNGRSFSTEELESVMDNQLPGTSEMVICSFEKGFHGRLFGSLSTTRSKPIHKLDIPAFNWPKAPFPDLKYPLEENKEANKAEESSCIEKFSQIVQEWQGKIAAVIIEPIQSEGGDNHASSDFFQKLREITIENGILMIVDEVQTGVGATGKMWAHEHWNLSNPPDLVTFSKKFQAAGFYYHDPKLQPDQPFRQFNTWCGDPSKALIAKVIYEEIVKHDLVTRTAEVGNYLFNRLEKLFEGKNYIQNLRGKGQGTYIAFDFGTSSERDSFLSRLRCNGANVAGCGDSAVRLRPSLTFEEKHADVLVSIFDKTLRQLYG